(19) United States
(12) Patent Application Publication
(10) Pub. No.: US 2024/0382206 A1
(43) Pub. Date: Nov. 21, 2024

(54) SURGICAL LEFT ATRIAL APPENDAGE CLOSURE DEVICES AND METHODS FOR DELIVERY

(71) Applicant: Conformal Medical, Inc., Nashua, NH (US)

(72) Inventors: Aaron V. Kaplan, Norwich, VT (US); Jacob Carl Hahn, Medford, MA (US); David A. Melanson, Hudson, NH (US); Carol Devellian, Topsfield, MA (US)

(21) Appl. No.: 18/664,100

(22) Filed: May 14, 2024

Related U.S. Application Data
(60) Provisional application No. 63/502,370, filed on May 15, 2023.

(51) Int. Cl. A61B 17/12; A61B 17/00

(52) U.S. Cl. CPC A61B 17/12122; A61B 17/12031; A61B 17/12145; A61B 17/12159; A61B 2017/00477; A61B 2017/12095

(57) ABSTRACT

Surgical devices and methods for excluding the left atrial appendage (LAA) via a thoracoscopic approach or at the time of open heart surgery. The device includes a trocar for accessing the LAA and a delivery system for implantation of the occlusion implant through the trocar. The occlusion implant can be an expandable metal clip or an expandable metal-framed occlusion device that includes a tissue scaffold. The trocar is placed through an opening in the tip of the LAA, then the occlusion implant is delivered through it and placed near the ostium of the LAA to seal it off from the left atrium. The hole in the tip of the LAA is then closed. Other associated surgical devices and methods are described.

### CROSS-REFERENCE TO RELATED APPLICATIONS

This application claims priority to U.S. Provisional Patent Application No. 63/502,370, titled SURGICAL LEFT ATRIAL APPENDAGE CLOSURE DEVICES AND METHODS FOR DELIVERY, filed on Oct. 19, 2022, which is incorporated by reference in its entirety.

### BACKGROUND

### Field

This development relates generally to excluding the left atrial appendage (LAA). In particular, surgical devices and methods for excluding the left atrial appendage (LAA), via a thoracoscopic approach or at the time of open heart surgery, are described.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for left atrial appendage (LAA) occlusion.

The following disclosure describes non-limiting examples of some embodiments. For instance, other embodiments of the disclosed systems and methods may or may not include the features described herein. Moreover, disclosed advantages and benefits can apply only to certain embodiments and should not be used to limit the disclosure.

Devices and methods for occluding the left atrial appendage are described herein. In some implementations, the techniques described herein relate to a device for closing off a left atrial appendage (LAA) of a patient. The device can include a proximal hub; one or more fronds extending distally from the proximal hub; one or more anchors disposed at the distal end of the one or more fronds, the one or more anchors configured to engage an ostium of the LAA; and a collar disposed around the one or more fronds. The collar may move distally along the one or more fronds, causing the one or more anchors to be pulled toward a central longitudinal axis into a closed position and close the ostium of the LAA.

In some implementations, the collar may lock the one or more fronds in the closed position. In some implementations, the one or more fronds may include a first bump feature. In some implementations, the collar may move distally along the one or more fronds and over the first bump feature. The first bump feature may retain the collar in a distal position relative to the first bump feature.

In some implementations, the one or more fronds may include a second bump feature disposed distally from the first bump feature. In some implementations, the collar may move distally along the one or more fronds and over the first bump feature, causing the one or more fronds to be pulled a first distance toward the central longitudinal axis and into a partially closed position. The collar may move distally along the one or more fronds and over the second bump feature causing the one or more fronds to be pulled a second distance toward the central longitudinal axis and into the closed position.

In some implementations, the one or more fronds can include at least two fronds or at least four fronds. In some implementations, the collar may include a notch configured to secure the collar to a delivery device. In some implementations, the proximal hub may include a pin that can be disposed proximal to the collar and may move distally into a central lumen of the collar, causing the one or more fronds to be deployed. In some implementations, the pin may include a step configured to prevent the pin from extending beyond the collar.

In some aspects, the techniques described herein relate to a method for occluding a left atrial appendage (LAA) of a patient. The method may include providing an implant within a delivery device. The implant may include: an expandable tubular body having a compressible open cell foam sidewall, an occlusive end, an open end, and a longitudinal axis extending therethrough; and a self-expandable support carried within the expandable tubular body, the self-expandable support including a plurality of struts forming a plurality of apexes. The method may also include creating an incision at a tail of the LAA of the patient; inserting the delivery device with the implant through the incision into the LAA; deploying the implant within the LAA to allow the self-expandable support and the tubular body to expand such that the foam sidewall contacts the wall of the LAA and provides a cushion between the support and the wall of the LAA; securing the implant; and closing the incision at the tail of the LAA.

In some implementations, the implant may also include a suture tether attached to the self-expandable support. The suture tether may collapse the support when tension is applied to the suture tether. In some implementations, the implant may include attaching the suture tether to the LAA. In some implementations, the delivery device may include a grappling hook device. The grappling hook device may include a plurality of hooks coupled to the plurality of apexes of the support. In some implementations, the grappling hook device may also include a plurality of spring elements attached to the plurality of hooks, the plurality of spring elements configured to extend radially and expand the self-expandable support.

In some implementations, the method may also include inverting the implant such that the occlusive end of the expandable tubular body passes through the open end of the expandable tubular body.

In some implementations, the techniques described herein relate to a method for occluding a left atrial appendage (LAA) of a patient. The method may include providing an implant within a delivery device. The implant may include a foam body. The method may also include: creating an incision at a tail of the LAA of the patient; inserting the delivery device with the implant through the incision into the LAA; deploying the implant within the LAA; securing the implant with the LAA; and closing the incision at the tail of the LAA.

In some implementations, the foam body may include a solid plug configured to self-expand and occlude the LAA.

In some implementations, the implant may also include a first support and a second support disposed within the foam body. The first support may include a first plurality of struts forming a first plurality of apexes. The second support may include a second plurality of struts forming a second plurality of apexes.

In some implementations, the implant may also include a suture that may encircle an exterior of an ostium of the LAA and may secure the implant within the ostium of the LAA. The foam body may include a cork shape that may be placed in the ostium of the LAA and may resist compression by the suture.

### BRIEF DESCRIPTION OF THE DRAWINGS

FIGS. 1 and 2 show respectively a cross-section of a heart and a left atrial appendage with an occlusion device therein.

FIGS. 3A-8D depict various embodiments of an occlusion device, having a foam and frame, and the features of which may be used with the various surgical devices and methods herein.

FIGS. 9A and 9B are schematics of the SLAAC-Clip/Delivery System assembly (100). FIG. 9A shows a schematic inside view of an SLAAC-Clip/delivery system (100) which is comprised of an SLAAC-Clip (200) and a delivery system (300). FIG. 9B shows the SLAAC-Clip (200) detached from the delivery system (300) and the delivery system (300) after releasing the clip (200).

FIGS. 10A-10C show the SLAAC-Clip in the open configuration (FIG. 10A) and closed configuration (FIG. 10B). The SLAAC-Clip has three parts, Fronds (215), Anchors (218), Collar (220) and Pin (230).

FIG. 10A shows the Fronds fully extended in the relaxed (open) configuration.

FIG. 10B shows the SLAAC-Clip is in the closed configuration with the Collar advanced and the anchors, which are embedded in the left atrial appendage (LAA) ostium, have been brought together to close off the LAA.

FIG. 10C shows a 3-dimensional view with 5 Fronds distributed evenly. In some embodiments, there can be anywhere from 2 to 10 Fronds.

FIGS. 11A-11G are schematics of the delivery system. FIG. 11A shows the delivery system with the SLAAC-Clip in place and constrained. This is how it can be provided for use.

FIG. 11B shows a side view of the delivery system which is comprised of the Outer Tube (330) and the Pincer(s) (340).

FIG. 11C shows a side view of delivery system with the Pincers depressed releasing the SLAAC-Clip. Note that the distal end of the Pincers are angulated to engage the Radial Notch of the Collar.

FIG. 11D shows a side view of the Outer Delivery Tube (330). The outer delivery tube can be a simple tube, with a central lumen which runs along the long axis of the device.

FIG. 11E shows cross-section A-a showing the distal region.

FIG. 11F shows cross-section B-b showing the middle region which has slits (325) which are designed to accommodate the Pincers.

FIG. 11G shows cross-section C-c showing the proximal region. Of note is the diameter of the central lumen (322) which has a reduced diameter designed to accommodate the inner tube (320) seen in FIG. 11A.

FIG. 12A shows a schematic of Inner Tube (320) which is a simple tube with a central lumen (322) as seen in Section A-a. At the distal end of the device the outer contour is threaded (325) in a manner to interact with the Threaded Channel (227) of the Collar (220) as discussed below.

FIG. 12B shows a schematic of Collar (220) which is shown in side and end views. In the embodiment shown there is a central lumen (222) that is in continuity with the central lumen of the inner tube (322). There is also a radial notch (225) which is designed to engage the Pincers as discussed above. Also shown is the pin (230) which is cylindrical in shape. Towards the proximal end there is a threaded central lumen which is designed to engage with the distal end of the Push Rod (315) as discussed below.

FIG. 12C shows a schematic of Push Rod which is a simple rod with a threaded element at the distal end (315).

FIGS. 13A-13J show a schematic of deployment of the SLAAC-Clip.

FIG. 13A shows the SLAAC-Clip/Delivery System initial configuration which is how it can be provided to the operator with the Fronds within the device constrained by the Inner Tube (320).

FIGS. 13B-D show the Fronds are expelled by advancing the Push Rod (310). The Push Rod, which is connected to the SLAAC-Clip Pin (230), can be advanced until the leading edge of the pin is at the distal extent of the Collar (220). The inner tube is then disengaged from the Collar by rotating via the threaded mechanism described above.

FIG. 13E shows the inner tube is then removed.

FIGS. 13F-13H depict the Fronds of the SLAAC-Clip being closed by withdrawing the Fronds through the Collar. The Fronds are locked in place by allowing the proximal end to take on its unconstrained configuration (which now occurs with the inner tube having been removed).

FIG. 13I shows, if the operator is now pleased with the position of the clip, it can be released by uncoupling the Push Rod.

FIG. 13J shows final release occurring with disengagement from the outer tube by actuating the Pincers (340).

FIGS. 14A and 14B shows a schematic depicting the SLAAC-Clip in the unconstrained (FIG. 14A) and Closed/Locked configuration (FIG. 14B).

FIGS. 14C-14E show an alternative embodiment in which the Fronds are shaped with an added Bump Feature (219) that allows for a Partially Closed as well as a Fully Closed configuration.

FIG. 15 shows a schematic of the Left Atrium and Left Atrial Appendage. The arrows indicate the plane of the ostium.

FIG. 16 shows the procedure is initiated with the surgeon making an incision in the tail of the left atrial appendage.

FIG. 17 shows the SLAAC-Clip/Delivery System as it is inserted through the incision.

FIG. 18 shows a purse string suture being placed around the SLAAC-Clip/Delivery System.

FIG. 19 shows the SLAAC-Clip/Delivery System is advanced towards the ostium.

FIG. 20 depicts the fronds being extruded from the Delivery System by advancing the Push Rod.

FIG. 21 depicts the fronds further extruded from the Delivery System.

FIG. 22 depicts the fronds fully extruded from the Delivery System. The inner tube is disengaged from the Delivery System By rotation.

FIG. 23 depicts the fronds fully extruded from the Delivery System with the inner tube removed.

FIG. 24 depicts how the assembly is now retracted.

FIG. 25 depicts the assembly now retracted.

FIG. 26 depicts as the assembly is further retracted.

FIG. 27 depicts the Frond Tips engaging the tissue around the ostium by further retraction.

FIG. 28 depicts the Ostium as it is closed by pulling the Frond System through the Collar.

FIG. 29 depicts the SLAAC-Clip locked in place by allowing the frond element to take its unconstrained configuration.

FIG. 30 depicts the push rod (310) disengaged from the SLAAC-Clip by rotation.

FIG. 31 depicts the push rod (310) withdrawn from the SLAAC-Clip.

FIG. 32 depicts the Outer Tube (340) disengaged from the SLAAC-Clip by actuating the pincer mechanism.

FIG. 33 depicts the procedure is completed by closing the left atrial appendage access site with the purse strings suture.

FIG. 34 depicts an alternative embodiment with a tether (400) connecting the Pin to the LAA wall with a pledgeted suture (410).

FIG. 35 depicts a slit in back of LAA.

FIG. 36 depicts a trocar with loaded closure implant in position outside LAA.

FIG. 37 depicts a trocar inserted through a puncture in either the side or tip of the LAA.

FIG. 38 depicts the implant deployed in LAA showing suture attachment if repositioning or retrieval is required.

FIG. 39 depicts the pusher catheter positioned for implant collapse, if needed.

FIG. 40 depicts the implant collapsed for repositioning or retrieval.

FIG. 41 depicts how once the implant is in proper location, required checks are conducted prior to implant release.

FIG. 42 depicts how the suture attachment (lasso) is removed and the incision is closed.

FIG. 43 depicts an option for securing any of the implants is to use a pledget mechanism during the suture closure of the appendage.

FIG. 44 depicts deployment of a CONFORMAL® left atrial appendage seal (CLAAS®) implant, such as the implant described with respect to FIGS. 3A-8D, from the LAA tip (as opposed to from the proximal (LA) side. It is attached to the delivery system from within the implant frame using a grappling hook.

FIG. 45 depicts the steps needed to collapse the implant for repositioning, if needed. The pusher is advanced up to the tip of the grappling hook assembly.

FIG. 46 shows the implant being collapsed for either repositioning or removal.

FIG. 47 depicts an alternate version where the grappling hooks are spring elements which also act to help expand the implant frame, useful for deployment of Nitinol in a cold heart.

FIG. 48 depicts a procedure for implantation of a CLAAS® implant, such as the implant described with respect to FIGS. 3A-8D, from the distal tip of the LAA where resheathing of the implant is done by inverting the implant. In this image, the pusher is placed adjacent to the LA tip from within the frame.

FIG. 49 depicts the outer trocar placed within the frame, coaxial to the pusher.

FIG. 50 depicts inversion of the implant for repositioning or retrieval.

FIG. 51 depicts a marshmallow-shaped LAA closure implant being delivered through a trocar from the distal LAA tip.

FIG. 52 depicts deployment of the closure implant.

FIG. 53 depicts one of multiple options for securing the implant within the LAA following deployment of the implant and removal of the trocar. These include suturing through foam near the ostium of the LAA, incorporating a tail tether, or a combination of anchoring methods. Additionally, pledgets can be added outside the LAA for extra security.

FIG. 54 depicts the final deployed implant, secured in place.

FIG. 55 depicts a modified version of the a CLAAS® implant, such as the CLAAS implant described with respect to FIGS. 3A-8D. The open distal end of a transcatheter delivered CLAAS® implant is replaced by a second hub. This allows the first hub—positioned near the LAA ostium—to provide a flat face in the LAA while the second hub—positioned near the LAA tip—allows for attachment during delivery and repositioning or retrieval. The anchors may be similar to those of the implant described with respect to FIGS. 3A-8D.

FIG. 56 depicts a modified CLAAS implant where the hub of the internal frame does not face the left atrium (LA). It faces the LAA tip to facilitate delivery and repositioning or retrieval. The tip of the frame is splayed outward, to facilitate anchoring of the implant. The open end of the frame is covered by foam which can also have an ePTFE cover, with or without perforations.

FIG. 57 depicts a variation of the dual-hub implant shown in FIG. 55. In this version, a distal anchor that extends outside the LAA tip is employed. This type of anchor can be added to any of the implant designs in this disclosure.

FIG. 58 depicts another embodiment having a slit placed in the distal LAA tip in a cold heart to open it up to allow delivery of the implant.

FIG. 59 depicts placement of the implant which already in its expanded shape. Alignment arms on the delivery system help to find the proper location for the suture lasso which is placed around the implant from outside the LAA.

FIG. 60 depicts how this concept is different from other LAA closure methods which expand to fill a pressurized LAA. In this embodiment the LAA is brought down in contact with the implant from exterior to the LAA. This is fixed in place and can also bring anchors in touch with the interior LAA wall for additional fixation.

FIG. 61 depicts the final implant placement with the tip of the LAA sutured closed while also showing how the implant material is highly conformable to different shapes of LAA ostia.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

Surgical approaches for occluding the LAA, such as via a thoracoscopic approach or at the time of open heart surgery, are described herein. The system may include an LAA occlusion device or implant, a trocar, and a delivery system for implantation of the LAA occlusion device through the trocar. Various embodiments of the system and the LAA occlusion device are described. Surgical approaches involving a surgical clip device, a purse string/lasso device, a grappling hook device, a marshmallow-like device, various implants with modified frames, and a champagne cork-like device are described.

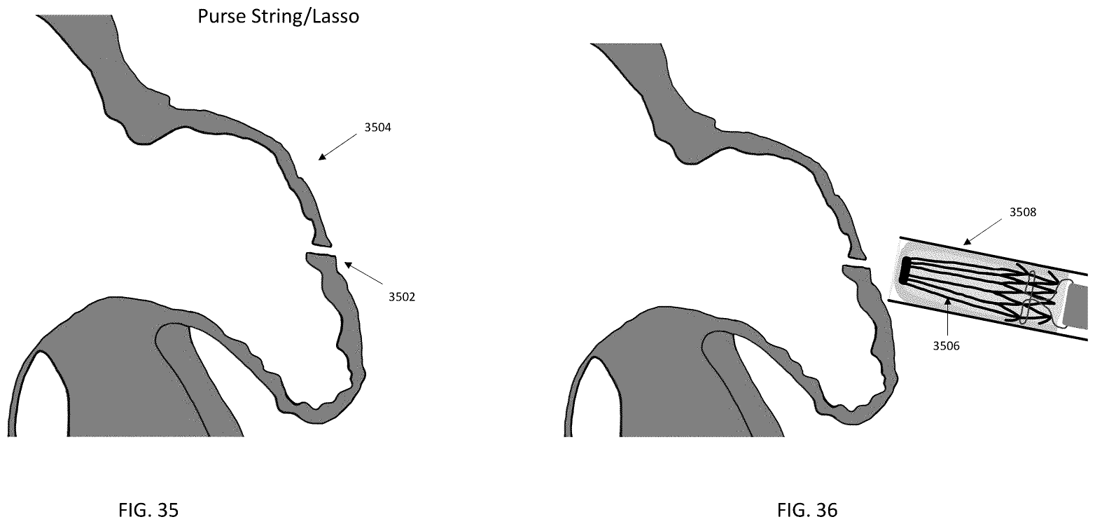

Further, LAA occlusion devices having one or more similar structural features of some transcatheter delivered LAA occlusion devices may be used in the surgical approaches described herein. For example, the various systems, devices and methods for the surgical approaches described herein, such as the various devices used in the surgical procedures shown and described with respect to FIGS. 35-57, may include the same or similar features and/or functionalities as other transcatheter LAA occlusion systems or devices, such as those described, for example, in U.S. application Ser. No. 17/675,779 entitled “MULTIFUNCTIONAL LEFT ATRIAL APPENDAGE (LAA) OCCLUDER,” and filed on Feb. 18, 2022, in U.S. application Ser. No. 17/183,160 entitled “DEVICES AND METHODS FOR EXCLUDING THE LEFT ATRIAL APPENDAGE” and filed on Feb. 23, 2021, in U.S. application Ser. No. 16/782,871 entitled “DEVICES AND METHODS FOR EXCLUDING THE LEFT ATRIAL APPENDAGE” and filed on Feb. 5, 2020, in U.S. application Ser. No. 15/795,083 entitled “DEVICES AND METHODS FOR EXCLUDING THE LAA” and filed on Oct. 26, 2017, and/or as described in U.S. application Ser. No. 15/969,654 entitled “DEVICES AND METHODS FOR EXCLUDING THE LAA” and filed on May 2, 2018, the entire disclosure of each of which is incorporated herein by reference for all purposes and forms a part of this specification.

### A. LAA Occlusion

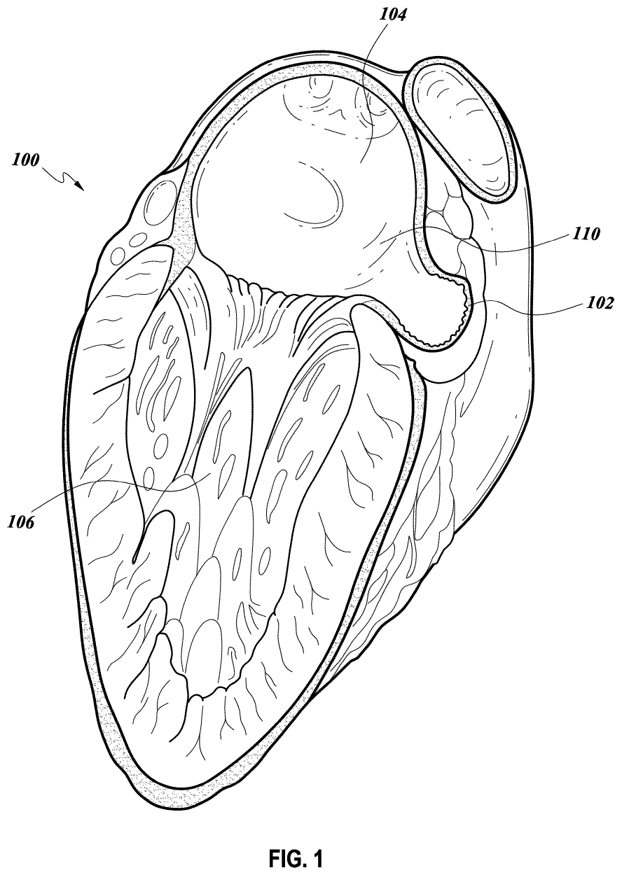

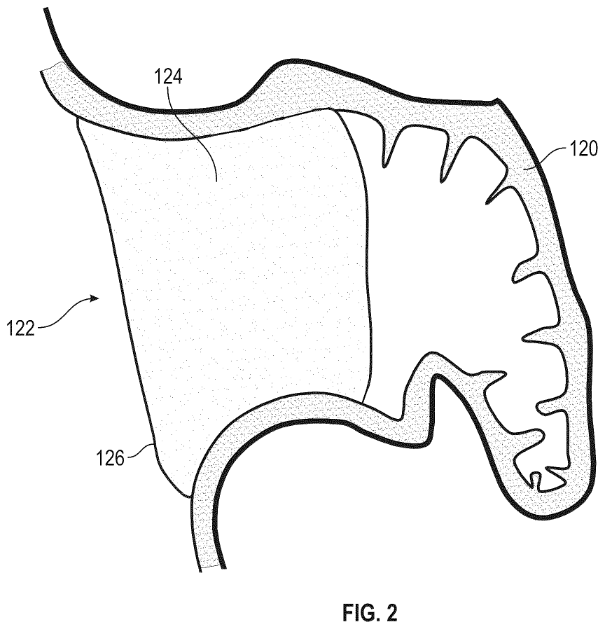

A cross-section of a human heart 100 is shown in FIG. 1 with a left atrial appendage (LAA) 102, which is a cavity emanating from the left atrium (LA) 104. FIG. 2 shows one embodiment of an LAA occlusion device 124 occluding the LAA 102. The LAA 102 is quite variable in shape in all dimensions. If the heart is not beating normally, a condition called atrial fibrillation, blood within the LAA becomes stagnant which promotes clot formation. If blood clots within the LAA, the clots may pass from the LAA 102 to the LA 104, to the left ventricle 106 and out of the heart 100 into the aorta. Vessels that bring blood to the brain branch off the aorta. If the clot passes to the brain via these vessels, it may get stuck and occlude a small vessel in the brain which then causes an ischemic stroke. Strokes have severe morbidities associated with them. The opening of the LAA 102 to the LA 104 is called an ostium 110. The ostium 110 is oval, highly variable and dependent on loading conditions, e.g., left atrial pressure. An object of the LAA occlusion devices described herein is to occlude the ostium 110 thereby sealing off the LA 104 from the LAA 102.

The devices and related methods are described herein in connection with use in occluding, i.e. excluding, a left atrial appendage (LAA) via a surgical procedure. The system may include a trocar for accessing the LAA and a delivery system for implantation of the occlusion implant through the trocar. The occlusion implant can be an expandable metal clip or an expandable metal-framed occlusion device that includes a tissue scaffold. The trocar is placed through an opening in the tip of the LAA, then the occlusion implant is delivered through it and placed near the ostium of the LAA to seal it off from the left atrium. The hole in the tip of the LAA is then closed. Other various embodiments and features thereof are described.

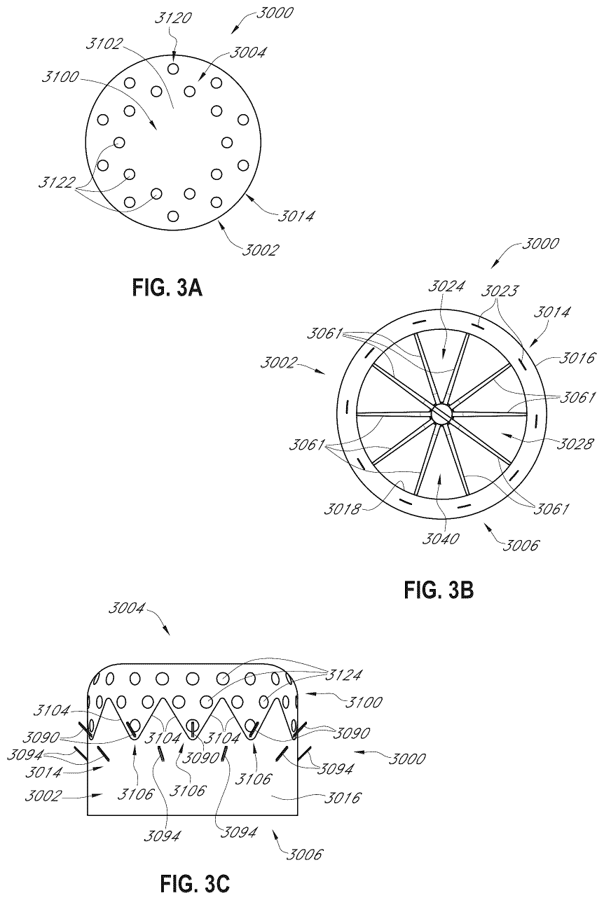

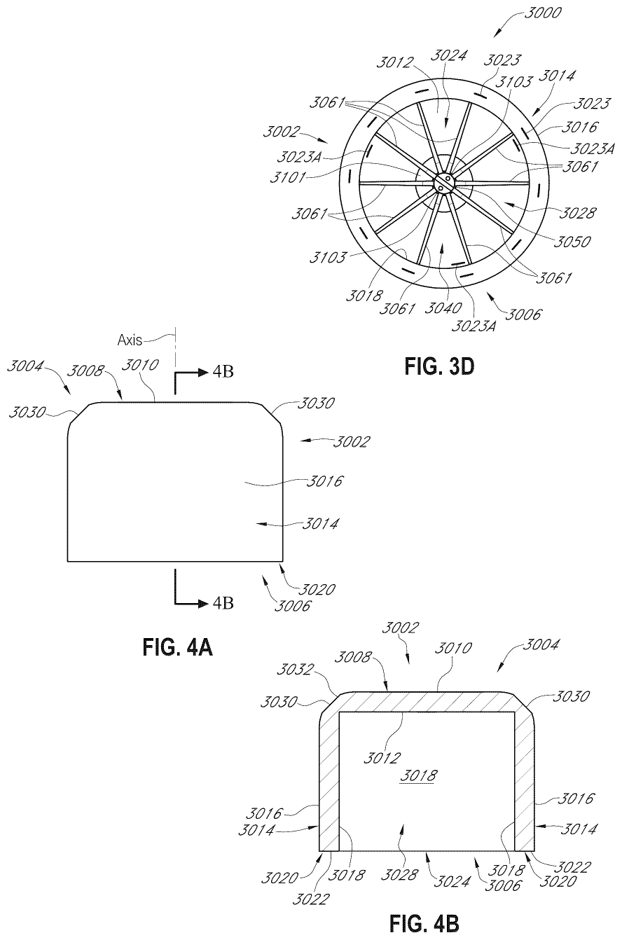

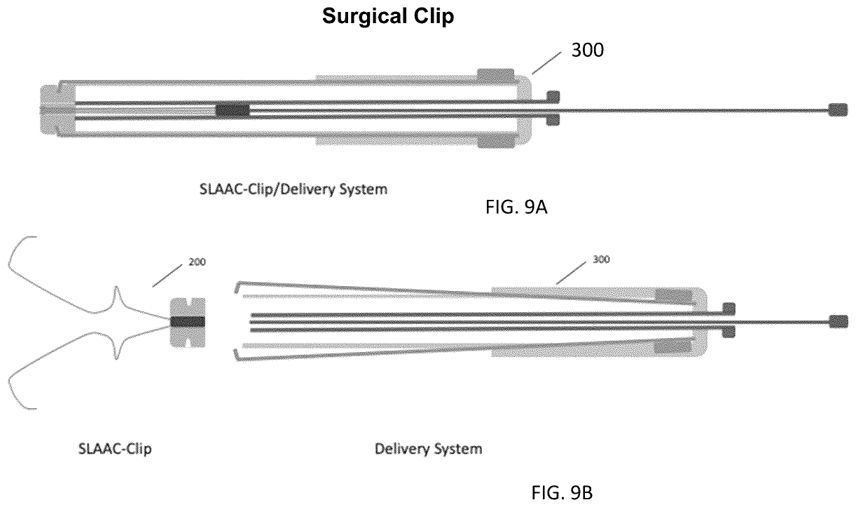

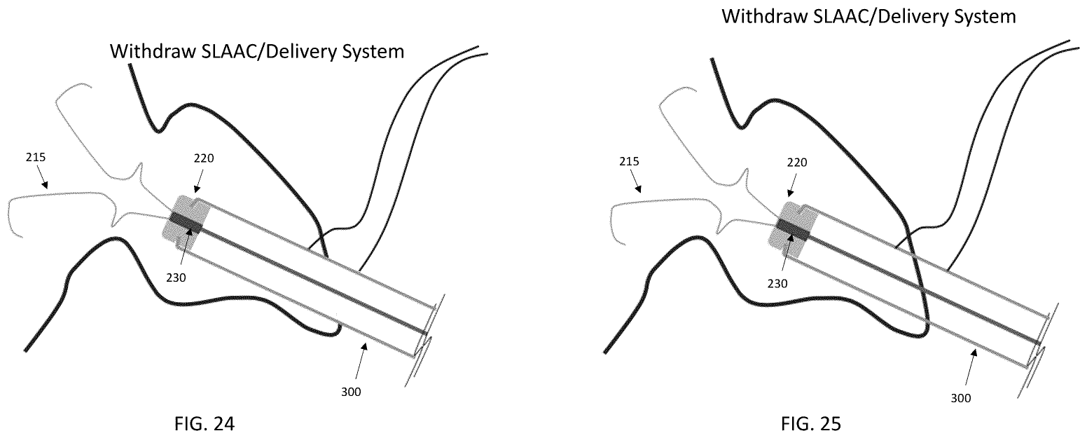

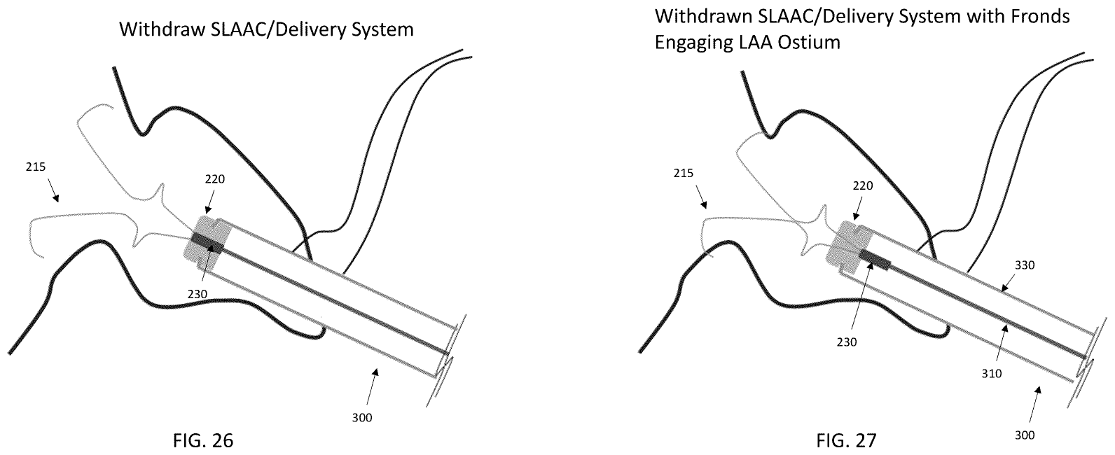

Various embodiments of LAA occlusion devices that may be used in the surgical approaches described herein are shown and described with respect to FIGS. 9A-61, including a surgical clip device, a purse string/lasso device, a grappling hook device, a marshmallow-like device, various implants with modified frames, and a champagne cork-like device. Other embodiments of LAA devices having a compressible outer foam and supporting inner frame with anchors and that may be used in the surgical approaches described herein are shown and described with respect to some of the above figures and as well as in FIGS. 3A-8D. Numerous options for occluding the LAA of the heart directly during open heart surgery or via a thorascopic approach are described. The LAA can be entered externally from what would be considered the distal end of the LAA during an interventional closure procedure (e.g. the tip of the LAA).

For surgically placed LAA closure, transesophageal echocardiography (TEE) may be conducted at the start of the surgical procedure, prior to placing the patient on bypass. This can be done to confirm there is no thrombus in the LAA and to size the appendage in multiple views in a pressurized, beating heart. Certain embodiments of LAA closure procedures described herein occur in a beating, pressurized heart after the heart has been re-started and brought to body temperature.

Alternatively, the LAA may be closed while the patient is on bypass. In certain embodiments, expansion of an implant frame (e.g., a Nitinol) may be challenging in a cold heart. During bypass surgery, hypothermia of the entire body is induced to preserve the heart muscle, brain, and other vital organs. The heart is cooled to well below room temperature and Nitinol implants, as commonly designed, may not fully expand after release from the delivery catheter. The hypothermia induced during cardiac surgery is typically <20° C. whereas current-day Nitinol implants are typically designed to come to their fully expanded diameter at body temperature, approximately 37° C. Nitinol at body temperature is in its austenitic state and displays good rigidity and is very springy, which is a property referred to as superelasticity. When Nitinol implants are collapsed within a catheter, they briefly convert to a martensitic phase, induced by the stress placed on the implant frame to collapse it. While some current-day devices can partially expand at temperatures as low as room temperature (˜25° C.), they typically do not come to their full diameter due to the typically available Austenitic finish (Af) temperatures, the temperature above which Nitinol is in a rigid state. Certain embodiments described herein may overcome these challenges by heating the implant or utilizing other implantable metals which can have consistent properties across a wider temperature range such as MP35N, Elgiloy, stainless steel, etc.

The LAA occlusion devices described herein for the surgical approach can include multiple functionalities. For example, the devices may include open internal spaces or cavities that provide a location for the incorporation of additional electronic devices or systems such as pacers, biosensors, drug delivery systems, defibrillators, pressure sensors, motion sensors, and/or any other suitable devices or systems. The LAA occlusion devices can include anchoring systems or components, such as an anchor or frame, that can also function as a staging or docking point to secure the additional devices or systems within the LAA occlusion devices.

The anchor or frame can be formed of one or more metals. For example, the anchor or frame can be formed of one or more of Nitinol, MP35, Elgiloy, stainless steel, or any other suitable metal. In some embodiments, the anchor or frame is fabricated from a laser-cut Nitinol tube. In some embodiments, the anchor is fabricated from woven or braided metals. The anchor or frame can be covered by a scaffold, such as a foam body. The foam body may be cylindrical in shape. The foam may be tubular in shape. The body can include an open cell foam material. There may be an expanded Polytetrafluoroethylene (“ePTFE”) layer on the proximal end.

In certain embodiments, the device can include an open or openable proximal end that can allow for delivery of the additional devices or systems (e.g., pacing devices or systems, defibrillator devices or systems, sensing devices or systems, drug delivery devices or systems, etc.) into the LAA occlusion device and which proximal end may then be closed to occlude the LAA opening. In other embodiments, the device can include one or more openings within the scaffold and/or frame of the device that can allow for delivery of additional devices or systems (e.g., pacing devices or systems, defibrillator devices or systems, sensing devices or systems, drug delivery devices or systems, etc.) through the LAA occlusion device. In other embodiments, additional devices or systems (e.g., pacing devices or systems, defibrillator devices or systems, sensing devices or systems, drug delivery devices or systems, etc.) may be deployed within the LAA before the LAA occlusion device or in combination with the LAA occlusion device. For example, additional devices or systems (e.g., pacing devices or systems, defibrillator devices or systems, sensing devices or systems, drug delivery devices or systems, etc.) may be coupled to the LAA occlusion device prior to delivery to LAA. In certain embodiments, these additional devices or systems (e.g., pacing devices or systems, defibrillator devices or systems, sensing devices or systems, drug delivery devices or systems, etc.) can be placed in patients having other cardiac diseases in addition to atrial fibrillation in order to address those other cardiac diseases by adding additional functionality to the LAA occlusion device, for example, to provide electrical isolation/ablation of the LAA, deliver drugs, provide pacing, and/or measure pressure.

In embodiments in which the additional devices or systems are pacing systems, the frame of the LAA occlusion device can provide an electrical connection between the pacing system and the atrial myocardium. For example, the portions of the frame, anchors, and/or alternative structures incorporated into the frame can contact the atrial myocardium in a penetrating or a non-penetrating manner to provide an electrical connection between the pacing system and the myocardium.

FIG. 2 shows the LAA occlusion device 124 placed within the LAA 102 at its opening to the LA 122. It is understood that the device 124 may have the same or similar features as other implantable “devices” or “implants” described herein, such as the device 3000, and vice versa. The device 124 may thus have an expandable foam body carrying a support structure or frame with anchors, as described herein, for example with respect to the device 3000 and FIGS. 3A-8D. The device 124 may have any of the features of the various implants described herein with respect to FIGS. 35-57.

The device 124 may be cylindrical in shape in an unconstrained expansion, but it may also be conical for example with its distal end smaller than the proximal end or reversed. It could also be oval in cross section to better match the opening of the LAA.

The device 124 is oversized radially in an unconstrained expansion to fit snuggly into the LAA and may be 5-50 mm in diameter depending on the diameter of the target LAA. The compliance and thickness of the foam are designed to provide a good seal against the tissue with minimal compression. While other devices require significant oversizing relative to the width of the LAA to obtain a seal, the implants described herein may require only ≤1 mm of oversizing. In some embodiments, the implant may require only ≤2 mm, ≤3 mm, or ≤4 mm, or ≤5 mm. In a free, unconstrained state, the axial length “L” of the plug is less than its outer diameter “D” such that the L/D ratio is less than 1.0. In some embodiments, this ratio may be greater than 1.0. The compliance of the foam material is designed such that it pushes on the walls of the LAA with sufficient force to maintain the device 124 in place but without overly stretching the LAA wall. The foam and/or skin also conforms to the irregular surfaces of the LAA as it expands, to provide a complementary surface structure to the native LAA wall to further enhance anchoring and promote sealing. Thus, the expandable foam implant described herein conforms to the native configuration of the LAA. In one embodiment, the structure of the foam may be fabricated such that squeezing axially on the opposing ends of the foam causes the foam to increase in diameter.

An outer ePTFE layer may be formed as a sheet. The sheet may have a wall thickness between 0.0001″ and about 0.003″ thick and serves to allow one to collapse and pull on the device 124 without tearing the foam material. The sheet may be formed from multiple sheets welded together using heat. It may also have one or more layers of electrospun material (e.g., electrospun ePTFE) to enhance tissue ingrowth. In other embodiments, an outer ePTFE layer may be formed from a tube with a diameter about the same diameter of the foam plug and a wall thickness between about 0.0001″ and about 0.003″ thick and serves to allow one to collapse and pull on the device 124 without tearing the foam material. The ePTFE material also serves as the blood contacting surface facing the LA 126 and has pores or nodes such that blood components coagulate on the surface and an intimal or neointimal covering of tissue grows across it and anchors tightly to the material. Pore sizes within the range of from about 4μ to about 110μ, ideally 5-35μ are useful for formation and adherence of a neointima.

The outer covering 126 may be constructed of materials other than ePTFE such as woven fabrics, meshes or perforated films made of FEP, polypropylene, polyethylene, polyester or nylon. The covering 126 should have a low compliance (non-elastic), at least longitudinally, be sufficiently strong as to permit removal of the plug, a low coefficient of friction, and be thromboresistant. The outer covering 126 serves as a matrix to permit plug removal as most foams are not sufficiently strong to resist tearing when pulled. The plug 124 can also be coated with or contain materials, such as PTFE. Such materials may enhance the plug's 124 ultrasonic echogenic profile, thromboresistance, and/or lubricity. The plug 124 can also be coated with or contain materials to facilitate echocardiographic visualization, promote cellular ingrowth and coverage.

The outer covering 126 may have holes in it to permit contact of the LAA tissue with the device 124 to encourage ingrowth of tissue into the foam plug pores and/or allow blood flow therethrough. These holes may be 1 to 5 mm in diameter or may also be oval with their long axis aligned with the axis of the foam plug, the length of which may be 80% of the length of the foam plug and the width may be 1-5 mm. The holes may be as large as possible such that the outer covering maintains sufficient strength to transmit the tensile forces required for removal. The holes may be preferentially placed along the device. In one embodiment, holes are placed distally to enhance tissue ingrowth from the LAA wall.

The device 124 or 3000 (as described below) may be anchored and secured in place in the LAA by anchoring features. In some embodiments, the device 124 or 3000 may also be anchored by tissue ingrowth.

Deployment of the occlusion device may be via direct surgical access or various minimally invasive access pathways (e.g. jugular vein). For example, the area overlying the xiphoid and adjacent costal cartilage may be prepared and draped using standard techniques. A local anesthetic may be administered and skin incision may be made, typically about 2 cm in length. The percutaneous penetration passes beneath the costal cartilage, and a sheath may be introduced into the pericardial space. The pericardial space may be irrigated with saline, preferably with a saline-lidocaine solution to provide additional anesthesia and reduce the risk of irritating the heart. The occlusion device may thereafter be introduced through the sheath, and through an access pathway created through the wall of the LAA. Closure of the wall and access pathway may thereafter be accomplished using techniques understood in the art.

Various features for LAA occlusion may be included in the LAA occlusion devices, systems, and methods described herein, such as those described, for example, in U.S. patent application Ser. No. 15/290,692, filed Oct. 11, 2016 and titled DEVICES AND METHODS FOR EXCLUDING THE LAA, and in U.S. patent application Ser. No. 14/203,187, filed Mar. 10, 2014 and titled DEVICES AND METHODS FOR EXCLUDING THE LAA, the entire disclosure of each of which is hereby expressly incorporated by reference for all purposes and forms a part of this specification. The embodiments described in the sections below may include the same or similar features and/or functionalities as the embodiments described above, and vice versa, except as otherwise noted or indicated by context.

B. LAA Occlusion Device with Compressible Foam Body and Compliant Frame

FIGS. 3A-8D show an embodiment of an LAA occlusion device 3000 that may be used in the surgical approaches described herein. The device 3000 described herein may have the same or similar features and/or functionalities as other LAA occlusion devices described herein, and vice versa. Any of the features of the device 3000 described with respect to FIGS. 3A-8D may therefore apply to features of the devices described with respect to FIG. 2, such as the device 204, or to any other devices described herein, and vice versa.

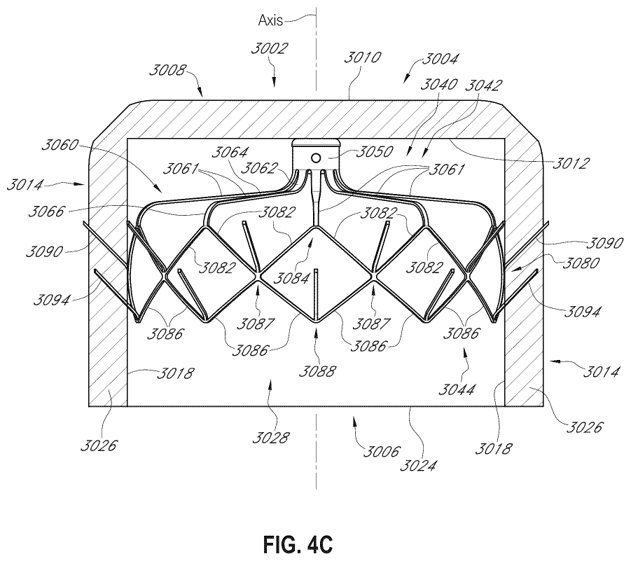

FIGS. 3A-3C show the LAA occlusion device 3000 having a scaffold or foam body 3002, an expandable support or frame 3040, and a proximal cover 3100. FIG. 3D shows the LAA occlusion device 3000 additionally having an interior cover 3101 and proximal markers 3023A. FIGS. 4A-4C show the foam body 3002, with the body 3002 shown in cross-section in FIGS. 4B and 4C. FIG. 4C additionally includes the full view (i.e. non-cross section) of the frame 3040. The device 3000 is shown in an expanded configuration in these figures. The device 3000 has a longitudinal axis as shown, which may be defined by the foam body 3002, as further described.

The body 3002 is formed from a compressible material, such as foam. The body 3002 may be a foam formed from reticulated (e.g. net-like) polycarbonate polyurethane-urea. The body 3002 may be cut, formed or assembled into a cup shape, as further described. The body 3002 may have a thickness and compressibility sufficient to engage the surrounding tissue and conform to the anatomic irregularities under radial force applied by the inner frame, as further described. The use of a compressible material such as foam for the body 3002 provides a complete seal of the LAA and superior performance for LAA occlusion over existing devices, as further described. The structure of the foam of the body 3002 comprises a three-dimensional network of interconnected reticulations, spaced apart to form a network of interconnected open pores, as further described. The reticulations can carry a coating, such as PTFE, while preserving the open pores, as further described.

The foam material of the body 3002 has a high porosity. “Porosity” as used herein has its usual and customary meaning and refers to open void content between the interconnected reticulations of the foam. The porosity of the body 3002 may be at least about 65%, at least about 70% at least about 75%, at least about 80%, at least about 85%, at least about 90%, at least about 95% or more. The porosity may be within the range of approximately 90-95%. The porosity may be approximately 90%. The porosity may be approximately 95%. The porosity may be 90%, 91%, 92%, 93%, 94%, or 95%. The high porosity promotes quick and tenacious tissue ingrowth, allows it to be compressed into a small catheter, and/or allows blood to pass if the implant embolizes, among other advantages.

The foam body 3002 has pores or cells formed between the interconnected reticulations of the foam material. The foam body 3002 has cells with sizes in the range of from about 250 μm to about 500 μm. The foam may have a cell size from about 125 μm to about 750 μm, from about 175 μm to about 650 μm, from about 200 μm to about 600 μm, from about 225 μm to about 550 μm, from about 275 μm to about 450 am, less than 125 μm, or greater than 750 μm. These sizes may refer to the size of the cell prior to application of any coating, such as PTFE. The cell size may thus change, e.g. decrease, after application of the coating. The desired porosity and/or cell size may be determined based on allowing the passage of blood while blocking debris of a size capable of potentially causing ischemic stroke. The allowable size of such debris may drive the selection of the particular porosity and/or cell size. For example, the cell size from about 250 μm to about 500 μm may be based on prevention of debris of a particular size from passing through the body 3002.

In an embodiment, the foam body 3002 is made from a non-resorbable, reticulated, cross-linked, polycarbonate polyurethane-urea matrix, structurally designed to support fibrovascular tissue ingrowth, with a fully interconnected, macroporous morphology with over 90-95% void content and cell sizes ranging from 250 to 500 m.

The body 3002 has a proximal end 3004 and a distal end 3006. In some embodiments, the axial length of the device 3000 from the proximal end to the distal end in a free, unconstrained state is 20 mm. As used herein, the “free, unconstrained” state, and the like, refers to a state of the device 3000 without any external forces applied to the device 3000 other than a normal or reactive force from a surface (e.g. table top) on which the device 3000 is placed. In some embodiments, this axial length may be from about 10 mm to about 30 mm, from about 12 mm to about 28 mm, from about 14 mm to about 26 mm, from about 16 mm to about 24 mm, from about 18 mm to about 22 mm, or about 20 mm. The body 3002 may have any of these lengths regardless of outer diameter of the body 3002.

The proximal end 3004 of the body 3002 has a proximal end wall or face 3008. The proximal face 3008 faces generally toward the LA when the device 3000 is implanted into the LAA. The device 3000 may be implanted off-axis, as further described, in which case the proximal face 3008 may not reside at a perpendicular to a longitudinal axis of the LA. The proximal face 3008 thus provides a closed proximal end 3004 of the body 3002. The closed proximal end 3004 is configured to span the ostium but the porosity, as further described, is sufficient to permit the passage of blood while blocking debris of a size capable of potentially causing ischemic stroke. This membrane may be formed by the body 3002 and/or the cover 3100. In some embodiments, the proximal face 3008 or portions thereof may be open. For example, there may not be a proximal face 3008, there may be a partial proximal face 3008, there may be a proximal face 3008 with portions removed, etc. In some embodiments, the proximal face 3008 or portions thereof is/are not included and any opening or openings is/are covered by the cover 3100. The size of any such openings in the proximal face 3008 may be driven by the desired size of embolic debris to be prevented from escaping the LAA, as further described.

The proximal face 3008 is flat or generally flat and generally perpendicular to the longitudinal axis of the device 3000. The proximal face 3008 has a circular or generally circular shape as viewed from the proximal end 3004 in an unconstrained expansion. In some embodiments, the proximal face 3008 may be flat, rounded, segmented, angled with respect to the longitudinal axis, other shapes, or combinations thereof. The proximal face 3008 may have a non-circular, polygonal, other rounded shape, other shapes, or combinations thereof, as viewed from the proximal end 3004.

The proximal face 3008 has an outer surface 3010 and an opposite inner surface 3012. The outer surface 3010 faces proximally away from the device 3000 and the inner surface 3012 faces distally toward the frame 3040. The surfaces 3010, 3012 may define outer and inner sides of the proximal face 3008. The thickness of the proximal face 3008 may be measured axially between the outer surface 3010 to the inner surface 3012. This thickness in a free, unconstrained state (e.g. uncompressed and expanded) may be from about 0.5 mm to about 5 mm, from about 1 mm to about 4 mm, from about 2 mm to about 3 mm, about 2.5 mm, or 2.5 mm. In some embodiments, the thickness may be less than 0.5 mm or greater than 5 mm. The thickness of the proximal face 3008 may be uniform or non-uniform. Thus, the thickness may be greater or smaller in different regions of the proximal face 3008.

The body 3002 includes a sidewall 3014 extending distally from the proximal face 3008. The sidewall 3014 extends circumferentially about a perimeter of the proximal face 3008 to form a closed cross-section (i.e. extends circumferentially 360 degrees about the axis). The sidewall 3014 extends axially to define a tubular body concentric about the longitudinal axis of the device 3000. The longitudinal axis extends through a geometric center of the tubular body defined by sidewall 3014. The sidewall 3014 is tubular or generally tubular, e.g. cylindrical, along the axis. In some embodiments, the sidewall 3014 may be conical or frustoconical, for example where the proximal end is wider than the distal end or vice versa. The sidewall 3014 may have an outer profile at the proximal end thereof, and as viewed from the proximal or distal end, to match that of the outer perimeter of the proximal face 3008.

In some embodiments, the cross-section of the sidewall 3014 may not be closed, for example where there are openings in the sidewall 3014. Thus, cross-sections taken at various locations along the longitudinal axis may or may not show a closed section. In some embodiments, the sidewall 3014 may be non-tubular, non-cylindrical, non-circular, polygonal, other rounded shapes, other shapes, or combinations thereof. In some embodiments, as shown, the sidewall 3014 may extend continuously for the entire length from the proximal end 3004 to the distal end 3006. In some embodiments, the sidewall 3014 may not extend continuously for the entire length from the proximal end 3004 to the distal end 3006. For example, the sidewall 3014 may include a plurality of disconnected sections, such as annular portions of the sidewall, located and spaced along the longitudinal axis and connected to the frame 3040.

The sidewall 3014 has an outer surface 3016 and an opposite inner surface 3018. The outer surface 3016 faces radially outward from the axis. The inner surface 3018 faces radially inward toward the axis. The thickness of the sidewall 3014 may be measured radially between the outer surface 3016 to the inner surface 3018. This thickness in a free, unconstrained state (e.g. uncompressed) may be from about 0.5 mm to about 5 mm, from about 1 mm to about 4 mm, from about 2 mm to about 3 mm, about 2.5 mm, or 2.5 mm. In some embodiments, the thickness may be less than 0.5 mm or greater than 5 mm. The thickness of the sidewall 3014 may be uniform or non-uniform. Thus, the thickness may be greater or smaller in different regions of the sidewall 3014. The thickness of the sidewall 3014 may be the same or different as the thickness of the proximal face 3008. In some embodiments, the thickness of the proximal face 3008 is 2.5 mm and the thickness of the sidewall 3014 is 2.5 mm. In some embodiments, the thickness of the proximal face 3008 is about 2.5 mm and the thickness of the sidewall 3014 is about 2.5 mm.

The sidewall 3014 has a distal free end 3020 having a distal surface 3022. The distal surface 3022 is flat or generally flat and perpendicular to the longitudinal axis of the device 3000. In some embodiments, the distal surface 3022 is non-flat, angled with respect to the axis of the device 3000, curved, rounded, segmented, other shapes, or combinations thereof.

The body 3002 may have a distal opening 3024. The opening 3024 is formed by the distal free end 3020 of the sidewall 3014. The opening 3024 is at a distal end of an internal central volume or cavity 3028 of the body 3002 that is formed at least partially by the sidewall 3014, the proximal face 3008 and/or the shoulder 3030. The frame 3040 may reside within the cavity 3028, as further described. The distal opening 3024 may be completely open. In some embodiments, the distal opening 3024 may be mostly open, partially open, or closed, for example where the body 3002 has a distal face similar to the proximal face 3008 to enclose or partially enclose the cavity 3028.

The body 3002 has a shoulder 3030, shown as a bevel, that extends between the proximal face 3008 to the sidewall 3014. The shoulder 3030 may be an intersection of a proximal end of the sidewall 3014 and the proximal face 3008. The shoulder 3030 extends circumferentially about the entire perimeter of the intersection. The shoulder 3030 has an outer surface 3032. The outer surface 3032 may be a beveled surface. The outer surface 3032 is flat or generally flat in an axial direction. The outer surface 3032 extends circumferentially about the entire perimeter of the shoulder 3030. In some embodiments the shoulder 3030 and/or outer surface 3032 may be non-flat, rounded, other shapes in an axial direction, or combinations thereof. The shoulder 3030 and/or outer surface 3032 may extend circumferentially less than the entire perimeter of the shoulder 3030. The thickness of the shoulder 3030 may be measured inward perpendicularly to the outer surface 3032. The thickness of the shoulder 3030 may be the same as the thicknesses of the proximal face 3008 and/or the sidewall 3014, as described herein. In some embodiments, the thickness of the shoulder 3030 may be different from the thicknesses of the proximal face 3008 and/or the sidewall 3014. The shoulder 3030 may function as a recapture ramp, to facilitate drawing the implant proximally into the deployment catheter.

The compressibility of the body 3002 contributes to the superior sealing capability of the device 3000. The foam may be compressible to provide a larger radial “footprint” and spread out the radial forces from struts on the frame 3040, as further described. The foam body 3002 may have a compressive strength of at least 1 pound per square inch (psi) or within a range of about 1 psi to about 2 psi, or no more than about 2 psi. The “compressive strength” here refers to the pressure to compress the foam to 50% strain. With some foam materials for the body 3002, the pressure may not change from 50% strain through at least 80% strain, and the relation of pressure versus strain may be flat or generally flat. Thus, even with thicker foams for the body 3002, the body 3002 will not exert much more outward force on the tissue due to the increased thickness by itself. In an embodiment, the foam body 3002 is a reticulated, cross-linked matrix having at least about 90% void content, an average cell size within the range of from about 250-500 microns, a wall thickness of at least about 2 mm and a compressive strength of at least about 1 psi. In an embodiment, the body 3002 is formed from a foam material having or substantially having the material properties indicated in Table 1. In some embodiments, the body 3002 is formed from materials described in, for example, U.S. Pat. No. 7,803,395, issued Sep. 28, 2010, and titled “Reticulated elastomeric matrices, their manufacture and use in implantable devices,” or U.S. Pat. No. 8,337,487, issued Dec. 25, 2012, and titled “Reticulated elastomeric matrices, their manufacture and use in implantable devices,” the entire disclosures of which are incorporated herein by reference.

TABLE 1
Example material properties for an embodiment of foam
material that may be used for the foam body 3002.

| Material Property | Value | |
|---|---|---|
| Permeability | 311 | Darcy |
| Average Cell Size | 377 | μm |
| Density | 2.7 | lb/ft³ |
| Compressive Strength | 1.1 | psi |
| Tensile Strength Parallel | 68 | psi |
| Tensile Strength Perpendicular | 32 | psi |

| | |
|---|---|
| Elongation Parallel | 219% |
| Elongation Perpendicular | 243% |

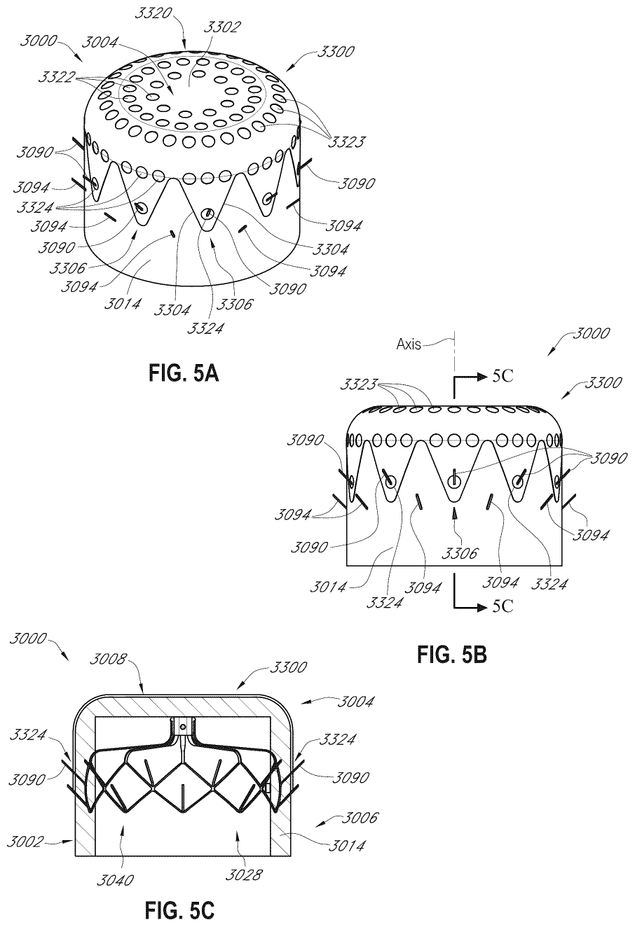

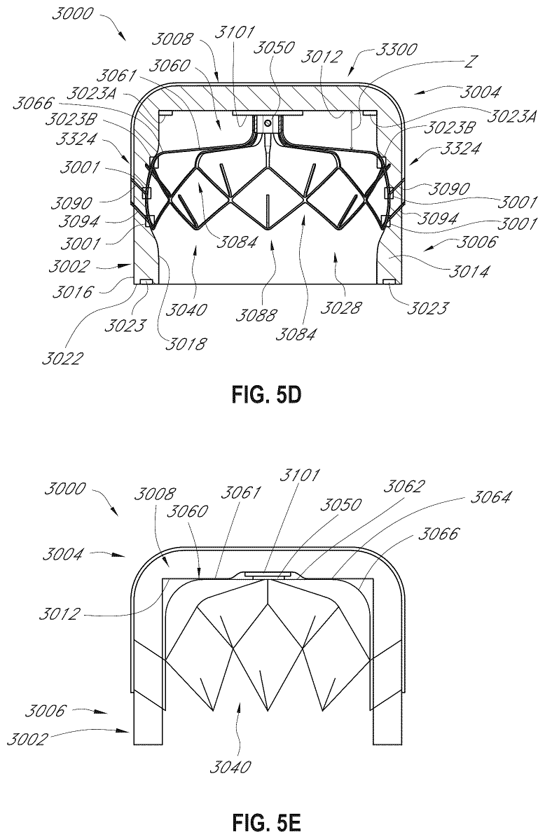

The device 3000 may include markers 3023 (see FIGS. 3B and 5D; for clarity only some of the markers 3023 are labelled in the figures) to facilitate visualization during delivery. The markers 3023 may be radiopaque marker bands sewn into the distal free end 3020 of the body 3002. The markers 3023 may be for visualization using fluoroscopy imaging of the distal end 3006 of the device 3000 during delivery. There may be a series of the markers 3023 located circumferentially along the distal surface 3022 of the body 3002 (for clarity, only some of the markers 3023 are labelled in FIG. 3B). In some embodiments, the markers 3023 may additionally or alternatively be located in other areas of the body 3002 and/or on other parts of the device, such as the cover 3100 or frame 3040.

In some embodiments, four platinum iridium (PtIr) radiopaque (RO) tubular markers 3023 are sewn onto the distal end 3006 of the foam body 3002 to enable visualization of the distal edge of the device 3000 under fluoroscopy. In some embodiments, a PtIr marker 3023 is attached to the foam body 3002 at the location of the proximal shoulder 3030 to use as a marker during recapture of the device 3000. Visualization of the proximal and/or distal markers 3023 may facilitate with identifying the amount of recapture. If the device 3000 is recaptured up to but not including the anchors proximal 3090 inside the access sheath, the device 3000 can be redeployed and reused. If the proximal anchors 3090 are recaptured into the access sheath, the device 3000 may be removed and discarded due to permanent deformation of the anchors 3090. In some embodiments, other materials may be used for the markers 3023, such as gold or other suitable materials.

As shown in FIGS. 3D and 5D, the device 3000 may include one or more markers 3023A. As one example only, there are three markers 3023A shown. In some embodiments, there may be one marker 3023A. There may be two, four, five or more markers 3023A. In some embodiments, there is one proximal marker 3023A and ten of the distal markers 3023. The markers 3023A may have the same or similar features and/or functionalities as other markers described herein, for example the marker 3023, and vice versa, except as otherwise noted. The markers 3023A may be located at or near the proximal end of the device 3000. As shown, the markers 3023A are located on an inner surface 3012 of the proximal end 3004 of the foam body 3002. The markers 3023A may be located at or near an inner surface of a shoulder 3030 (see FIG. 4B) of the foam body 3002. The markers 3023A may be distributed circumferentially, for example equidistant or equiangular, relative to each other, or they may be at different relative distances from each other. They may be radially located at the same or different location relative to each other. In some embodiments, there is only one marker 3023A. There may be one proximal marker 3023A and four of the distal markers 3023. The one or more markers 3023A may be on the inside, outside, or within the foam body 3002, or combinations thereof. The one or more markers 3023A may be located on or at the distal surface 3022 of the foam body 3022. The markers 3023A may be elongated circumferentially as shown. In some embodiments, the markers 3023A may be linear when the device 3000 is viewed from a particular angle, such as a side view. The markers 3023A may be aligned or oriented in the same or similar orientation, or in different orientations. Some, none, or all of the markers 3023A may be oriented circumferentially, laterally, axially (for example along an inner surface 3018 of the sidewall 3014), other orientations, or combinations thereof.

As further shown in FIG. 5D, there may be one or more markers 3023B. The one or more markers 3023B may have the same or similar features and/or functionalities as the other markers described herein, such as the marker 3023 or 3023A and vice versa, except as otherwise noted. The markers 3023B may be located along the sidewall 3014 of the body 3002. There may be one or more markers 3023B located along an inner surface 3018 of the sidewall 3014.

As shown, two markers 3023B are visible on either side of the interior of the foam body 3002. The markers 3023B are attached through the foam and around the frame 3040. The marker 3023B may be attached, for example sutured, around a proximal face 3060 member of the frame 3040, such as one of the struts 3061. The marker 3023B may be attached to the frame 3040 just proximally of one of the proximal apexes 3084 of the frame 3040, for example at an outer curved portion 3066 of the strut 3061. There may be only one marker 3023B, or two, three, four or more markers 3023B. There may be one of the markers 3023B for each strut 3061. The markers 3023B may be used additionally to connect the frame 3040 with the foam body 3002. The markers 3023B may be sutures as described herein.

The one or more markers 3023A and/or 3023B at or near the proximal end of the device 3000 provide various desirable features. For instance, the marker 3023A at the shoulder 3030 facilitates visualization of the device 3000 during and after implantation. The typically non-circular shape of the opening of the LAA (ostium) may compress the proximal end 3004 of the device and cause the proximal end 3004 to protrude slightly in the proximal direction. However, the shoulder 3030 may provide a location for the marker 3023A where linear bulging of the foam body 3002 in the proximal direction is reduced or prevented. Thus, the marker 3023A in that location can provide a more useful visualization of the positioning of the device 3000 and reduce complexity. For example, in some embodiments, the marker 3023A at the shoulder 3030 (e.g. on an inner surface as shown) may be particularly useful during delivery, allowing for delivery using fluoroscopy imaging only without the need for echo or other ultrasound imaging. The one or more markers 3023B may provide similar benefits.

As further shown in FIGS. 3D and 5D, the device 3000 may include an inner cover 3101. The inner cover 3101 may have the same or similar features and/or functionalities as the cover 3100 (described in further detail below, see section “Proximal Cover”), except as otherwise described. The inner cover 3101 may be a cover for the hub 3050 (see, e.g., FIGS. 4C and 7A-8C). The inner cover 3101 may be formed from expanded Polytetrafluoroethylene (“ePTFE”). The inner cover 3101 may be a separate portion of the same material as the proximal cover 3100.

The inner cover 3101 may be located between the foam body 3002 and the frame 3040. As shown, the inner cover 3101 is located between the inner surface 3012 of the foam body 3002 and a proximal end of the hub 3050 of the frame 3040. The inner cover 3101 may be circular or other shapes. The inner cover 3101 may have an area sufficient to provide a barrier in between the hub 3050 and the proximal end 3004 of the foam body 3002. In some embodiments, the inner cover 3101 may extend radially to an outer circumference of the hub 3050, or it may extend radially to the sidewall 3014 such as to an inner surface 3018 of the foam body 3002, or to any radial locations in between. The inner cover 3101 may have a diameter from about 4 mm to about 22 mm, from about 5 mm to about 15 mm, from about 6 mm to about 10 mm, about 8 mm, or 8 mm. The inner cover 3101 may be flat or generally flat. The inner cover 3101 may have a thickness from about 0.0001″-0.0020″, from about 0.0002″-0.0010″, about 0.0005″, or 0.0005″ thick. The inner cover 3101 may include one or more openings 3103 such as holes therethrough. The inner cover 3101 may include two holes 3103 to receive therethrough a tether 3240. The two holes 3103 in the cover 3101 may align the tether 3240, such as a suture, that extends distally into the hub 3050 through one hole 3103 in the inner cover 3101 and exits proximally back out of the hub 3050 through the other hole 3103 of the inner cover 3101.

The inner cover 3101 may prevent the hub 3050 and/or other features of the frame 3040 from directly contacting the foam material. The cover 3101 may protect the integrity of the foam body 3002 from stresses that may be imparted by the hub 3050 on the foam material. This protection may be desirable for example during loading, deployment, retrieval, re-deployment, etc. of the device 3000. The inner cover 3101 may prevent or reduce damage to the foam body 3002 from the hub 3050.

The foam body 3002 may be attached to various features of the device 3000. The body 3002 may be attached to the frame 3040 at numerous points, including for example the center of the proximal end of the frame 3040, as further described herein. Attachment can be done using suture, such as polypropylene monofilament suture, although other methods known in the art such as adhesive bonding could be utilized. The proximal row of proximal anchors 3090 may be individually attached to (e.g. inserted through) the foam body 3002 to prevent relative movement between the foam body 3002 and the frame 3040. In other embodiments, the foam body 3002 could be formed around the endoskeleton so that the metallic frame is within the foam body 3002, eliminating the need for a secondary attachment step. Attachment of the body 3002 to the frame 3040 promotes retrieval without damage to the foam body 3002, among other advantages. The attachment also ensures that a bumper 3026, further described herein, extends beyond the frame 3040 at all times, including during initial exposure of the device 3000 upon proximal retraction of the delivery sheath.

As shown in FIG. 5D, the device 3000 may include one or more attachments 3001. The attachments 3001 may connect the frame 3040 with the foam body 3002. The attachments 3001 may be sutures. Other suitable attachment structures may be used, including staples, ties, wires, components of the frame 3040, other mechanical attachments, adhesives, other suitable means, or combinations thereof. The attachments 3001 may extend around the frame 3040 and through the foam body 3002, for example through the sidewall 3014.

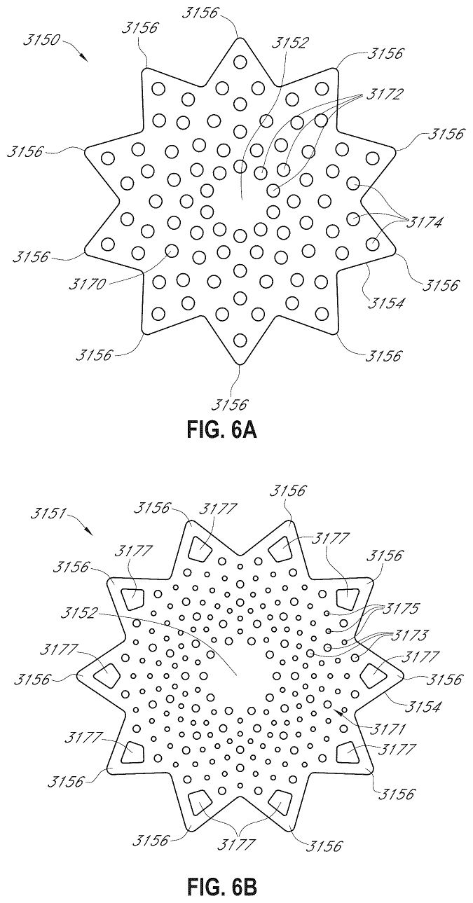

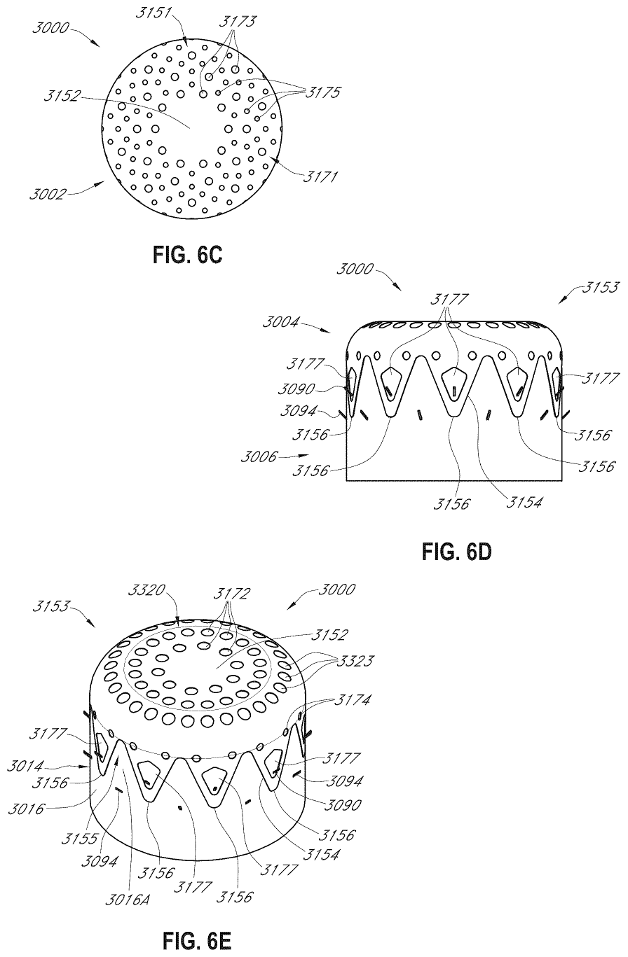

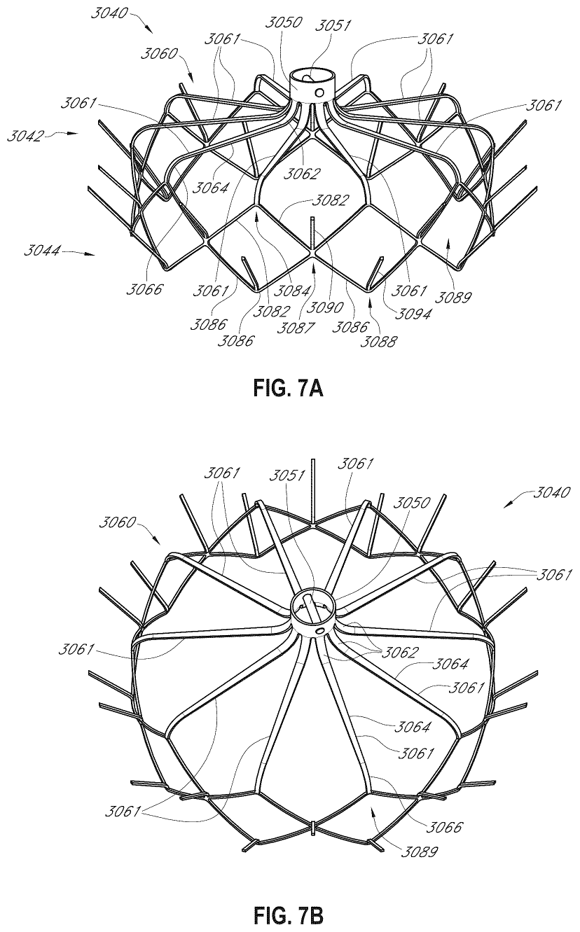

As shown, four attachments 3001 are visible in FIG. 5D. There are two proximal attachments 3001 and two distal attachments 3001 visible. The proximal attachments 3001 are each located at the base of a respective proximal anchor 3090. The distal attachments 3001 are each located at the base of a respective distal anchor 3094. There may be one, two, three, four, five, six, seven, eight, or more attachments 3001. There may be twenty attachments 3001. There may be one of the attachments 3001 for each anchor 3090, 3094 of the device 3000. The attachments 3001 may each be located at a proximal apex 3084 or at a distal apex 3088 of the frame 3040, as further described herein, for example with respect to FIG. 7A. For example, the attachments 3001 may be wrapped around one or more of the struts 3082, 3086, as further described herein. The attachments 3001 may locally compress the foam body 3002 at and/or around the location of attachment. The attachment 3001, such as a suture, may extend from within the cavity 3028, through the foam body 3002, exit the foam body 3002 and extend along the outer surface 3016 of the foam body 3002, extend back into and through the foam body 3002 into the cavity 3028, and be tied or otherwise connected together around the frame 3040. In some embodiments a similar routing of the attachments 3001 may be used with the attachment 3001 tied or otherwise connected together around and outside the foam body 3002. In some embodiments the attachments 3001 may also extend through the cover 3300, or other covers as described herein. The attachments 3001 may extend through the material of the cover 3300. The attachments 3001 may extend through openings in the cover 3300, such as the side openings 3324, or windows 3177 (see, e.g., FIGS. 6B-6E). As shown, the proximal attachments 3001 may extend through the foam body 3002 and through openings in the cover 3300, and the distal attachments 3001 may not extend through the cover 3300 but only through the foam body 3002.

The foam body 3002 may include a coating. In some embodiments, there may not be a coating. In embodiments with a coating, the coating is applied to the interconnected reticulations of the foam material. The body 3002 may be coated with pure polytetrafluoroethylene (PTFE). The PTFE coating minimizes the thrombogenicity of the LA surface, while also reducing the friction of the foam body 3002 against the delivery system to facilitate ease of deployment and retrieval. The body 3002 may be coated with conformable, vacuum deposited, pure PTFE. In addition or alternatively, the body 3002 may be coated with a coating other than PTFE. The coating, whether PTFE or otherwise, may be about 0.5 μm thick, and covers at least a portion of the surface of the interconnected reticulations of the foam without occluding the pores. The coating may be applied to some or all of the foam body 3002. The coating may be applied to some or all of the outer surfaces of the foam body 3002.

In some embodiments, the thickness of the coating is from about 0.1 μm to about 1 μm, from about 0.2 μm to about 0.9 μm, from about 0.3 μm to about 0.8 μm, from about 0.4 μm to about 0.7 μm, about 0.4 μm to about 0.6 μm, or about 0.5 μm thick. In some embodiments, greater or smaller thicknesses of the coating may be applied. The coating has a uniform or substantially uniform thickness. In some embodiments, the coating may have a non-uniform thickness. For example, the portion of the body 3002 facing the LA when implanted, such as the proximal face 3008 and/or shoulder 3030, may have a thicker coating relative to a coating along the sidewall 3014 of the body 3002. In some embodiments, the outer surface 3010 of the proximal face 3008 has a PTFE coating and the proximal face 3008 also has a ePTFE cover 3100.

The coating is applied using a vapor deposition process. In some embodiments, the coating is applied through coating, vapor deposition, plasma deposition, grafting, other suitable processes, or combinations thereof. The coating is applied to the outer surfaces 3010, 3032 and 3016 of, respectively, the proximal face 3008, the shoulder 3030 and the sidewall 3014. In some embodiments the coating is applied to the outer surfaces 3010, 3032 and only partially on the outer surface 3016. In some embodiments the coating is applied to outer and inner surfaces of the body 3002.

In some embodiments, other biocompatible, thromboresistant and/or lubricious materials could be applied to the surface(s) of the foam body 3002 and/or the cover 3100. These materials may encourage tissue ingrowth. Such materials may include, for example, heparin, albumin, collage, polyethylene oxide (PEO), hydrogels, hyaluronic acid, materials that release nitric oxide, oxygen, nitrogen, amines, bioabsorbable polymers, and other biomaterials, pharmacologic agents, and surface modification materials. Additionally, the surface(s) of the body 3002 could be roughened, textured, or otherwise modified or coated to promote healing or to make it more echogenic.

The device 3000 may include a cover 3100, which may be an ePTFE cover as further described. Other embodiments for this outer cover 3100 are described herein, for example the cover 3101, 3300, 3150, 3151, etc. The various embodiments of the cover may have the same or similar features and/or functionalities as each other, except as otherwise noted. The cover 3100 may have a series of openings. In some embodiments, the cover 3100 may be solid and not have any openings. In some embodiments, the cover 3100 may only have openings to receive anchors and/or a tether therethrough, as further described herein. In some embodiments, the device 3000 may include an inner cover such as an inner cover 3101, as shown and described with respect to FIG. 3D.

The outer cover 3100 is a generally flat material applied over and covering at least a portion of the body 3002. The cover 3100 is on the proximal end 3004 of the device 3000. The cover 3100 covers the proximal face 3008 of the body 3002 and at least part of the sidewall 3014. The cover 3100 covers a proximal portion of the sidewall 3014. The cover 3100 has a proximal surface 3102 that at least partially faces the LA when implanted. The cover 3100 has an outer edge 3104 forming outer vertices 3106 (for clarity, only some of the outer edges 3104 and outer vertices 3106 are labelled in the figures). In some embodiments, the cover 3100 may cover only the proximal face 3008 or portions thereof. In some embodiments, the cover 3100 may extend over more of the sidewall 3014, such as the middle or distal portion thereof, or the entire sidewall 3014.

The cover 3100 may have a thickness measured perpendicularly from the proximal surface 3102 to an opposite distal surface of the cover 3100 that faces the body 3002. The cover 3100 may have a thickness of 0.001″ (inches). In some embodiments, the cover 3100 may have a thickness from about 0.00025″ to about 0.005″, from about 0.0003″ to about 0.004″, from about 0.0004″ to about 0.003″, from about 0.0006″ to about 0.002″, from about 0.0008″ to about 0.0015″, or about 0.001″. In some embodiments, the cover 3100 may have a thickness of 0.0005″. In some embodiments, the cover 3100 may have a thickness from about 0.0002″ to about 0.0008″, from about 0.0003″ to about 0.0007″, from about 0.0004″ to about 0.0006″, or about 0.0005″.

The cover 3100 may be attached to the frame 3040 through the foam body 3002. The cover 3100 may in addition or alternatively be attached to the body 3002. The cover 3100 may be attached at least two or four or six or more of the outer vertices 3106. The cover 3100 may be attached to the frame 3040 and/or body 3002 at various locations, including at the outer vertices 3106, through the proximal surface 3100, at the proximal face 3008 of the body 3002, other locations, or combinations thereof. The cover 3100 may cover the entire foam body or some of the foam body may be directly exposed to the blood. For example, in some embodiments, the cover 3100 can be recessed proximally from a distal end of the sidewall 3014 by about 5 mm. In some embodiments, the cover 3100 can be recessed proximally from a distal end of the sidewall 3014 by between 1 mm and 15 mm, between 2 mm and 10 mm, between 3 mm and 8 mm, between 4 mm and 6 mm, or any other suitable distance. The cover 3100 is attached using mechanical attachments, such as sutures. In some embodiments, polypropylene 6-0 sutures are used throughout the device to attach the foam body 3002, proximal cover 3100, and RO markers 3023 to the foam body 3002 and/or frame 3040. In some embodiments, the cover 3100 is attached to the frame 3040 via standard braided or monofilament suture material, such as polypropylene, ePTFE, or polyester. In some embodiments, a polypropylene monofilament is utilized. Proximal anchors 3090 of the frame 3040 (further described herein) may extend through the outer vertices 3106 of the cover 3100. Such penetrating anchors 3090 may further secure the cover 3100 in place relative to the body 3002. In some embodiments, the cover 3100 may be attached to the various parts of the device 3000 with mechanical attachments, fasteners, adhesives, chemical bonds, other suitable techniques, or combinations thereof.

As shown, the cover 3100 is formed from expanded Polytetrafluoroethylene (“ePTFE”). An ePTFE cover 3100 provides many advantages. For example, the ePTFE cover 3100 may enhance the ability to recapture the device 3000 in vivo by distributing the proximal retraction forces applied by the catheter. The cover 3100 may be an ePTFE material approximately 0.001″ thick, with the appropriate porosity to encourage healing and minimize thrombus formation, similar to the underlying PTFE coated foam.

An ePTFE cover 3100 may assist in recapture of the implant into the access sheath while providing a smooth, thromboresistant surface which encourages tissue coverage and integration. The ePTFE may cover the entire proximal face and partially covers the sides, as shown in FIG. 3C. The ePTFE cover 3100 is fabricated from a previously laminated sheet comprised of two or more sheets of oriented material, offset to form a biaxially orientated material. Alternatively, one could use a tube, preferably biaxially oriented, that is then cut to form a sheet. The thickness of the final construct can be from 0.0005″-0.005″ but is preferably about 0.001″.

In some embodiments, the cover 3100 is fabricated from other thromboresistant, high strength, biocompatible materials, such as knitted or woven polyester fabrics, polypropylene, polyethylene, non-woven vascular scaffolds, porous films, or bioabsorbable scaffolds such as polylactic acid, polyglycolic acid, and co-polymers. The shape of the cover prior to attachment with the device 3000, such as shown in FIGS. 6A and 6B, minimizes wrinkling and provides a smooth surface following attachment to the implant. This shape may be a star shape, an outer pointed shape, or other shapes.

The cover 3100 may be perforated with a series of openings 3120 (for clarity, only some of the openings 3120 are labelled in the figures). The openings 3120 are perforations or holes formed in the cover 3100 via laser or mechanical cutting. The openings 3120 include proximal openings 3122 and side openings 3124 (for clarity, only some of the proximal openings 3122 and side openings 3124 are labelled in the figures). When the cover 3100 is assembled with the body 3002, the proximal openings 3122 are located over the proximal face 3008 and/or shoulder 3030, and the side openings 3124 are located over the sidewall 3014. In some embodiments, the cover 3100 includes forty proximal openings 3122. In some embodiments, the cover 3100 includes forty side openings 3124. The number of openings 3120 located over the proximal face 3008 and/or shoulder 3030 when assembled with the body 3002 may range from ten to eighty, from twenty to seventy, from thirty to sixty, from thirty five to fifty, or forty openings 3120. The number of openings 3120 located over the sidewall 3014 may range from ten to eighty, from twenty to seventy, from thirty to sixty, from thirty five to fifty, or forty openings 3120.

The openings 3120 may have a variety of sizes. The openings 3120 are 0.070″ in width, e.g. minor axis, or diameter for circular openings. The openings 3120 may have a width from about 0.010″ to about 0.200″, from about 0.020″ to about 0.150″, from about 0.030″ to about 0.110″, from about 0.040″ to about 0.100″, from about 0.050″ to about 0.090″, from about 0.060″ to about 0.080″, or about 0.070″. In some embodiments, the width may be less than 0.010″ or greater than 0.200″, such as 0.25″, 0.5″ or greater. These widths may apply to circular as well as non-circular shaped openings 3120.

In some embodiments, the openings 3120 may be various shapes. The openings 3120 may be elongated slots. The openings 3120 may extend radially along the cover 3100 from or near a center portion of the proximal surface 3102 toward and/or to the outer edge 3104. The openings 3120 may be annular openings extending circumferentially along the cover 3100 and having varying radial positions. The openings 3120 may be of uniform size and shape. Some of the openings 3120 may have varied sizes and/or shapes with respect to other of the openings 3120. The openings 3120 may have various distributions or concentrations about the cover 3100. For example, the openings 3120 may be more densely located in various areas, such as along the proximal surface 3102 that faces the LA, along the shoulder 3030, etc.

The openings 3120 enable blood to flow through the device 3000. The openings 3120 may allow blood to adequately flow through the device 3000 and thereby mitigate the risk of occlusion in the bloodstream should the device 3000 embolize within the vasculature system. In some embodiments, should the device 3000 embolize, it may act as a stationary filter at low pressures but may pass through the bloodstream at higher pressures. In some embodiments, the device 3000 allows for about two to about fourteen liters, from about four to about twelve liters, from about six to about ten liters, or from about eight liters per minute of blood to pass at ≤30 mmHg pressure drop to prevent shock in the event of a device embolization. In some embodiments, there are forty circular openings 3120 each having a diameter of 0.070″, and allowing for approximately eight liters per minute of blood to pass at <30 mmHg pressure drop. In some embodiments, the proximal end of the device 3000 may be a foam layer such as the foam proximal face 3008 or a membrane such as the cover 3100 or both, enclosing the cavity 3028 defined within the tubular side wall 3014 of the body 3002.

In one implementation, having both the foam proximal face 3008 and the cover 3100, the foam body 3002 has the open cell structure further discussed herein that can permit the passage of blood but block escape of embolic debris. The cover 3100 may be occlusive to blood flow, and is present to provide structural integrity and reduced friction for retracting the expanded body 3002 back into the deployment catheter. In one implementation, the cover 3100 is ePTFE in a form that is substantially occlusive to blood flow, as described. In this embodiment, the cover 3100 is therefore provided with a plurality of perfusion windows or openings 3120, so that blood can pass through the open cell foam and cover 3100 but the device 3000 still benefits from the other properties of the cover 3100.

In some embodiments, the device 3000 may allow for a particular flow rate of water at specified conditions, to test the perfusion performance of the device 3000. The device 3000 may have the foam body 3002 and cover 3100 configured to allow for a flow rate of water axially through the device 3000 of at least 2.8 liters per minute. The water may be at sixty-eight degrees Fahrenheit (F) or about sixty-eight degrees f and an upstream pressure of twenty-eight millimeters of Mercury (mmHg) or about twenty-eight mmHg. In some embodiments, the device 3000 may be configured to allow for flow rates under such conditions from about 2.8 liters to about 19.6 liters, from 4.2 liters to about 5.6 liters, from about 4.76 liters to about 5.6 liters, from about 5.6 liters to about 16.8 liters, from about 8.4 liters to about 14 liters, more than 2.8 liters, more than 5.6 liters, more than 8.4 liters, or more than 11.2 liters of water per minute.

In some embodiments, the foam and cover are configured to allow for a flow rate of water axially through the device of between 4.2 liters per minute and 5.6 liters per minute (for example, in embodiment of a 27 mm diameter implant), with the water at about sixty-eight degrees Fahrenheit (F) and an upstream pressure of about twenty-eight millimeters of Mercury (mmHg). In some embodiments, the foam and cover are configured to allow for a flow rate of water axially through the device of between 4.76 liters per minute and 5.6 liters per minute (for example, in embodiment of a 35 mm diameter implant), with the water at about sixty-eight degrees Fahrenheit (F) and an upstream pressure of about twenty-eight millimeters of Mercury (mmHg).

The particular flow rate may depend on the porosity of the foam body 3002 and the open area of the cover 3100. The particular flow rate may depend on the inner cover 3101 features as well. The cover 3100 may have particular percentages of the cover area open with the series of openings, as further described herein, to attain a particular desired flow rate. The flow rate of water at the specified conditions may be used to extrapolate or otherwise calculate the corresponding expected flow rate of blood in the body through the device 3000 should it embolize, as described herein. In some embodiments, the device 3000 may be configured to allow for a flow rate of blood axially through the device 3000 of at least 1 liter per minute (for example, in embodiment of a 27 mm diameter implant at room temperature with an upstream pressure of about 15 inches of water head). The device 3000 may allow for a cardiac output from about 4.2 to 8 liters per minute. The average body surface area is 1.6 square meters for females and 1.9 square meters for males. The device 3000 may allow for a cardiac index from about 2.2 to 5 or from about 2.6 to 4.2 liters per minute per square meter. The device 3000 may have these and other flow rate capabilities either aligned or approximately aligned with the direction of flow of the fluid, or off-axis where the device 3000 is angled with respect to the direction of flow of the fluid (a flow axis), as further discussed herein for example in the section “Off-Axis Delivery and Deployment.”

FIGS. 5A-5C depict an embodiment of the LAA occlusion device 3000 having another embodiment of a cover 3300. The device 3000 includes the foam body 3002 and the frame 3040, and features thereof, as described herein, and additionally includes the cover 3300. The cover 3300 may have the same or similar features and/or functionalities as the cover 3100, and vice versa. The cover 3300 is on the proximal end 3004 of the device 3000. The cover 3300 covers the proximal face 3008 of the body 3002 and a proximal part of the sidewall 3014. The cover 3300 has a proximal surface 3302. The cover 3300 has an outer edge 3304 forming a plurality of at least two or four or six or eight or ten or more outer vertices 3306 (for clarity, only some of the outer vertices 3306 are labelled in the figures). The cover 3300 is attached to the body 3002 at the outer vertices 3306. The proximal anchors 3090 extend through side openings 3324 in the outer vertices 3106 of the cover 3100.

The cover 3300 includes a series of openings 3320. The openings 3320 include proximal openings 3322, shoulder openings 3323, and the side openings 3324. The proximal openings 3322 are located over the proximal end 3004 of the body 3002. The shoulder openings 3323 are located over the shoulder 3030, e.g. a bevel, of the body 3002. The side openings 3324 are located over a proximal portion of the sidewall 3014 of the body 3002. The proximal anchors 3090 may extend through the side openings 3324 that are located in the outer vertices 3106. The openings 3320 may have the same or similar features and/or functionalities as the openings 3120, and vice versa. In some embodiments, the proximal anchors 3090 may extend through the cover 3300 material at or near the outer vertices 3106.

FIG. 6A shows another embodiment of a cover 3150 that may be used with the device 3000. The cover 3150 may have the same or similar features and/or functionalities as the cover 3100 and/or cover 3300, and vice versa. The cover 3150 may be used to cover the proximal face 3008 of the body 3002 and part of the sidewall 3014. The cover 3150 has a proximal surface 3152. The cover 3150 has an outer edge 3154 forming outer vertices 3156. The cover 3150 may be attached to the body 3002 at the outer vertices 3156. The proximal anchors 3090 may extend through the outer vertices 3156 of the cover 3100. The cover 3150 includes a series of openings 3170. The openings 3170 include proximal openings 3172 and side openings 3174 (for clarity, only some of the openings 3170, 3172, 3174 are labelled in the figures). When the cover 3150 is assembled with the body 3002, the proximal openings 3172 are located over the proximal end 3004 and the side openings 3174 are located over the sidewall 3014. As shown, the openings 3174 may be substantially uniformly located along the cover 3150 except for a center region of the proximal surface 3152.

FIG. 6B is a top view of another embodiment of a proximal cover 3151 that may be used with the various LAA occlusion devices described herein. FIG. 6C is a top view showing the cover 3151 assembled with the device 3000. The cover 3151 may have the same or similar features and/or functionalities as other covers described herein, such as the cover 3100 and/or cover 3300, and vice versa, except as otherwise noted. For example, the cover 3151 may include the proximal surface 3152 and outer edge 3154 forming outer vertices 3156.

The cover 3151 further includes another embodiment of a series of openings 3171. The openings 3171 include smaller openings 3175 and larger openings 3173. The openings 3175, 3173 may have the same or similar features and/or functionalities as other cover openings described herein, such as the openings 3120, 3122, 3124, 3320, 3322, 3324, 3170, 3172 and/or 3174, and vice versa. The smaller openings 3175 may be relatively smaller, in width and/or area, than the larger openings 3173. There may be openings with widths or areas smaller than that of the smaller openings 3175, larger than that of the larger openings 3173, or anywhere in between. As shown, the openings 3173, 3175 may be generally uniformly distributed about the proximal surface 3152 of the cover 3151. The openings 3173, 3175 may be circumferentially evenly spaced or approximately evenly spaced about the cover 3151.

There may be a variety of different quantities of each of the openings 3173, 3175. There may be a total of ten, twenty, thirty, forty, fifty, sixty, seventy, eighty, ninety, one hundred, one hundred fifty, two hundred, three hundred, four hundred, or more openings of the series of openings 3171, or any lesser, greater or in between number of openings. The series of openings 3171 may be holes as shown. They may have circular shapes. They may have other shapes, including non-circular, segmented, other shapes, or combinations thereof. The openings 3171 may all have the same general shape or different shapes. In some embodiments, there may not be any holes in the cover 3151.

When the cover 3151 is assembled with the foam body 3002, the large and small openings 3173, 3175 may be located over the proximal end 3004 and/or the sidewall 3014 of the foam body 3002. When assembled with the foam body 3002, on the proximal-facing portion of the cover 3151, there may be a collective total of one hundred forty or about one hundred forty openings 3173, 3175. On this proximal-facing portion of the cover 3151, there may be a collective total from about ten to about three-hundred, from about fifty to about two hundred fifteen, from about one hundred ten to about one hundred seventy, from about one hundred twenty to about one hundred sixty, from about one hundred thirty to about one hundred fifty, or from about one hundred thirty-five to about one hundred forty-five openings 3173, 3175. On this proximal-facing portion of the cover 3151, there may be from about thirty to about fifty, from about thirty-five to about forty-five, about forty, or forty of the larger openings 3173. On this proximal-facing portion of the cover 3151, there may be from about sixty to about one hundred forty, from about eighty to about one hundred twenty, from about ninety to about one hundred ten, about one hundred, or one hundred of the smaller openings 3175.

When assembled with the foam body 3002, on the portion of the cover 3151 located over and/or near the shoulder 3030, such as over the outer surface 3032 of the foam body 3002 (see, e.g., FIG. 4B), there may be from about five to about eighty, from about ten to about forty, from about fifteen to about thirty, about twenty, or twenty of the smaller openings 3175. In some embodiments, at this same portion of the cover 3151, there may be from about five to about eighty, from about ten to about forty, from about fifteen to about thirty, about twenty, or twenty of the larger openings 3173.

When assembled with the foam body 3002, on the portion of the cover 3151 located over and/or near the sidewall 3014, such as over the outer surface 3016 of the foam body 3002 (see, e.g., FIG. 4B), there may be from about five to about eighty, from about ten to about forty, from about fifteen to about thirty, about twenty, or twenty of the larger openings 3173. In some embodiments, at this same portion of the cover 3151, there may be from about five to about eighty, from about ten to about forty, from about fifteen to about thirty, about twenty, or twenty of the smaller openings 3175.

The larger and smaller openings 3173, 3175 may have a variety of different sizes, for example as described herein with respect to the openings 3122. In some embodiments, the openings 3173, 3175 may have diameters ranging from about 0.025 inches to about 0.040 inches. In some embodiments, the larger openings 3173 may be 0.040 inches or about 0.040 inches in diameter. The larger openings 3173 may be from about 0.030 inches to about 0.050 inches, or from about 0.035 inches to about 0.045 inches, in diameter. These values may also refer to the widths, for example maximum widths, of non-circular larger openings 3173. In some embodiments, the smaller openings 3175 may be 0.025 inches or about 0.025 inches, in diameter. The smaller openings 3175 be from about 0.015 inches to about 0.035 inches, or from about 0.020 inches to about 0.030 inches, in diameter. These values may also refer to the widths, for example maximum widths, of non-circular smaller openings 3175.

The series of openings 3171 may be configured to provide a desired amount of open area through the cover 3151. This open area refers to the total area of certain openings in the cover 3151. The cover 3151 may be covering a proximal face 3008 at the proximal end 3004 of the foam body 3002. The open area may refer to openings through the portion of the cover that is over the proximal face 3008 of the foam body 3002 when assembled with the foam body 3002. The series of openings in the various covers described herein may collectively provide the open area. For example, the series of openings 3171 in the cover 3151 over the proximal face of the foam may collectively provide an open area. This is the sum of the area of the openings in the cover 3151 over the proximal face. As further example, the open area may be the sum of the proximal openings 3122 of the cover 3100. As further example, the open area may be the sum of the proximal openings 3322 of the cover 3300.

The open area may be at least five percent of the area of the proximal face 3008 of the foam body 3002. The open area may be at least six, at least seven, at least eight, at least nine, at least ten, at least eleven, at least twelve, at least thirteen, at least fourteen percent, at least fifteen, at least sixteen, at least seventeen, at least eighteen, at least nineteen, at least twenty, at least twenty-five, at least thirty, at least forty, or at least fifty percent, of the area of the proximal face 3008. The open area may be from about one to about fifty percent, from about five to about twenty percent, from about eight to about fifteen percent, from about ten to about twelve percent, or about eleven percent, of the area of the proximal face 3008. The “area” of the proximal face 3008 is understood here to refer to an area equal to Pi×R², where R is the radius of the proximal face 3008 and extends perpendicularly from the longitudinal axis of the device 3000. Further, “R” may be measured to the inner boundary of the shoulder 3030, to the outer boundary of the shoulder 3030, or to the outer surface 3016 of the sidewall 3014. Further, as mentioned, some embodiments may not include a cover at all.

The cover 3151 may include one or more windows 3177. As shown, there may be ten windows 3177. There may be one window 3177 for each proximal anchor 3090. There may be four, six, eight, twelve, fourteen or more windows 3177, or any lesser or in between number. The windows 3177 may be openings in the cover 3151. The windows 3177 may be located at or near the outer edge 3154 of the cover 3151. The windows 3177 may be located along portions of the outer edge 3154, for example at or near the outer vertices 3156. The windows 3177 may have a shape conforming to the shape of the cover 3151 at the respective portions of the outer edge 3154. As shown, the window 3177 may be diamond or generally diamond shaped. The window 3177 may be square, rectangular, triangular, rounded, circular, segmented, flattened diamond, other polygonal shapes, other shapes, or combinations thereof. The cover 3150 may be attached to the body 3002 at the outer vertices windows 3177. The windows 3177 may have the same or similar feature and/or functionalities as the side openings 3324, described and shown in FIG. 5B. The proximal anchors 3090 may extend through the windows 3177 of the cover 3151 to retain the cover 3151 on the device 3000.

FIG. 6D-6E are side and perspective views, respectively, of another embodiment of a proximal cover 3153 shown assembled with the device 3000, that may be used with the various LAA occlusion devices described herein. The cover 3153 may have the same or similar features and/or functionalities as other covers described herein, such as the cover 3100, 3151, and/or cover 3300, and vice versa, except as otherwise noted. For example, the cover 3151 may include the proximal surface 3152, outer edge 3154 forming outer vertices 3156, and windows 3177.

The device 3000 with cover 3151 may have proximal anchors 3090 extending through the windows 3177. The proximal anchor 3090 may extend through the opening of the respective window 3177. The proximal anchor 3090 may extend through a distal portion of the window 3177, for example to contribute to securing the cover 3153 on the device 3000. The proximal anchors 3090 may extend through the window 3177 at a distal edge or distal vertex of the window 3177. In some embodiments, the proximal anchor 3090 may extend through the cover 3151 material, for example through material adjacent (such as distal) to the window 3177. In some embodiments, the proximal anchor 3090 may extend through various other locations within, adjacent or near the window 3177. Some of the proximal anchors 3090 may extend through first locations and other of the proximal anchors 3090 may extend through second locations of the cover 3153 different from the first locations. For instance, one or more anchors 3090 may extend through a first region of the window 3177, one or more other anchors 3090 may extend through a second region of the window 3177, still one or more other anchors 3090 may extend through other regions, such as through the cover 3153 material, etc.

The cover 3153 may include proximal vertices 3155. The proximal vertices 3155 may be formed by the outer edge 3154. The proximal vertices 3155 may be indentations along the outer edge 3154 of the cover 3153, for example angled as shown or other shapes, configurations, etc. The proximal vertices 3155 may define a region 3016A of the outer surface 3016 of the sidewall 3014. The region 3016A may be partially enveloped by the outer edge 3154 of the cover 3153. The region 3016A may receive one or more of the distal anchors 3094 therethrough. The distal anchor 3094 may extend through a distal portion of the region 3016A, or in other locations within, adjacent, or near the region 3016A. In some embodiments, the distal anchor 3094 may not extend through or near the region 3016. There may be multiple such regions 3016A of the foam body 3002 defined circumferentially about the device 3000 by the cover 3153.

The cover 3153 may include the series of openings 3320, for example as described with respect to FIG. 5A. The series of openings 3320 may include the proximal openings 3172, the shoulder openings 3323, and/or the side openings 3174. The cover 3153 may include different patterns, sizes, distributions, etc. of the openings 3320, for example as shown and described with respect to FIGS. 6B-6C.

The expandable and compliant support or frame 3040 is shown, for example, in FIGS. 3B, 3D, 4C and 5C-E. Further, FIGS. 7A and 7B are side and proximal perspective views, respectively, of the frame 3040 shown in a deployed configuration and in isolation from the rest of the device 3000. The frame 3040 provides a compliant structure with anchors to facilitate delivery, anchoring, retrieval and to enable the foam body 3002 to compress against the LAA tissue to facilitate sealing, among other things, as further described. The frame 3040 is located inside the cavity 3028 formed by the foam body 3002. In some embodiments, the frame 3040 may be located partially or entirely inside one or more portions of the body 3002, e.g. within the proximal face 3008 and/or the sidewall 3014, as further described. For example, the frame 3040 may be partially located within the sidewall 3014 as shown in FIG. 5C.

The frame 3040 has a proximal end 3042 and an opposite distal end 3004. The frame 3040 may be tubular, e.g. cylindrical, in a free, unconstrained state. Thus the width of the proximal end 3042 may be the same or similar to the width of the distal end 3004 in the free, unconstrained state. In some embodiments, the frame 3040 or portions thereof may be conical or frustoconical, e.g. where in the free, unconstrained state the width of the proximal end 3042 is greater than the width of the distal end 3004 or vice versa.

At the proximal end 3042, the frame 3040 has a proximal hub 3050, shown as a cylindrical nipple. The hub 3050 is a rounded, structural end piece. The hub 3050 may be tubular, e.g. circular and having the cylindrical shape as shown, or may be rounded, non-circular, segmented, other shapes, or combinations thereof. The hub 3050 extends axially and may have a central lumen. The hub 3050 may be wider than it is long, or vice versa. The hub 3050 is hollow and has a sidewall defining a space therethrough, such as a longitudinal opening. In some embodiments, the hub 3050 may be partially hollow, solid, or other configurations. The hub 3050 facilitates delivery and retrieval of the device 3000, as further described. The hub 3050 may provide a central structural attachment, as further described herein. The hub 3050 may be located within the cavity 3028 at a proximal end thereof. In some embodiments, the hub 3050 may be located partially or entirely within the foam body 3002, e.g. within the proximal face 3008.

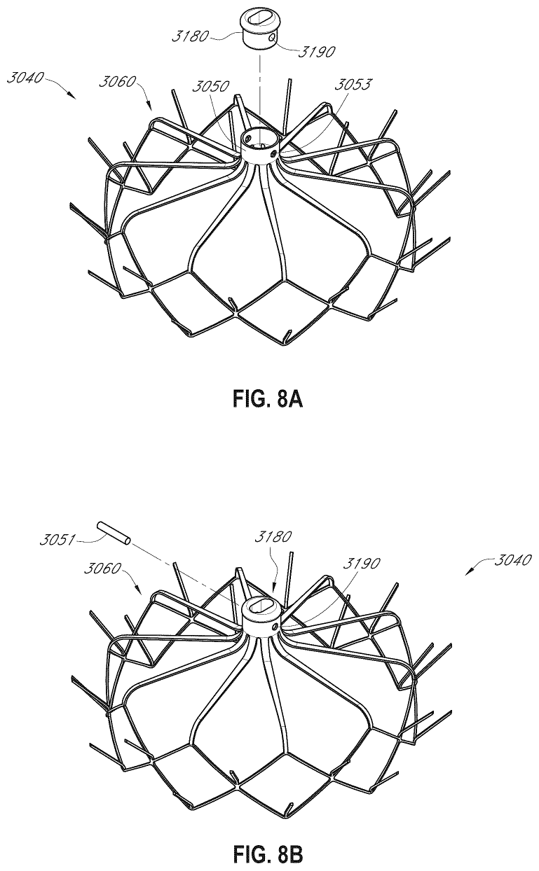

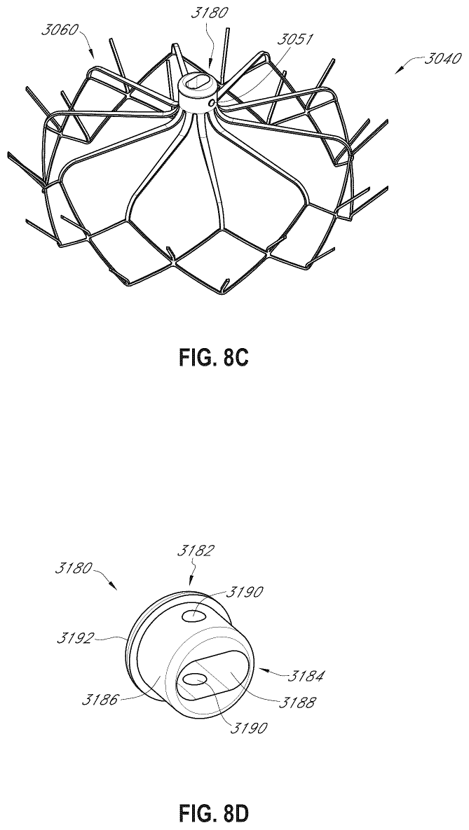

A pin 3051 is located within the hub 3050 (shown in FIGS. 7A and 7B). The pin 3051 is an elongated, rounded structural element extending laterally across the central lumen. “Lateral” here refers to a direction perpendicular or generally perpendicular to the longitudinal axis. The pin 3051 has a cylindrical shape. The pin 3051 provides a rounded outer surface configured to provide a smooth engagement surface with a tether, as further described. The pin 3051 provides a high strength connection with the frame 3040 to allow for pulling on the device 3000 with sufficient force to re-sheath the device 3000. The pin 3051 may be formed from Nitinol. The pin 3051 is secured across the width, e.g. diameter, of the proximal hub 3050. The pin 3050 may be secured at its two opposite ends with the sidewall of the hub. The pin 3051 is configured to be engaged by a tether 3240, which is wrapped around the pin 3051 in sliding engagement for temporary attachment to a delivery catheter, as further described. In some embodiments, the pin 3051 is assembled with a cap 3180, as further described herein, for example with respect to FIGS. 8A-8C.

The frame 3040 at the proximal end 3042 includes a proximal face 3060. The proximal face 3060 may be located within the cavity 3028 at a proximal end thereof. In some embodiments, the proximal face 3060 may be located partially or entirely within the foam body 3002, e.g. within the proximal face 3008 and/or sidewall 3014. The proximal face 3060 includes a series of recapture or reentry struts 3061. The struts 3061 are located at a proximal end of the cavity 3028. In some embodiments, the struts 3061 or portions thereof may be located partially or entirely within the foam body 3002, e.g. within the proximal face 3008 and/or sidewall 3014.

The struts 3061 are elongated structural members. The struts 3061 may have rectangular, circular or other shaped cross-sections. In some embodiments, the struts 3061 have a cross-section, e.g. rectangular, with a width that is greater than a thickness such that the struts 3061 are stiffer in one direction compared to another direction. This width may be in the lateral direction or a direction generally perpendicular to the longitudinal axis of the device 3000 when the device 3000 is in the expanded configuration, with the thickness perpendicular to the width. The struts 3061 may be less stiff in the direction of flexing or bending, for example to facilitate contraction and expansion of the device 3000 in the delivery and expanded configurations. The struts 3061 may be elongated pins. The struts 3061 may extend from the hub 3050, for example, and incline radially outwardly in the distal direction from the hub 3050. The struts 3061 may be attached inside, outside, and/or at the end of the sidewall of the hub 3050. The struts 3061 may be separate parts that are then attached to the hub 3050, for example welding, bonding, fastening, other suitable means, or combinations thereof. In some embodiments, some or all of the struts 3061 and the hub 3050 may be a single, continuous structure formed from the same raw material such as a laser cut hypotube. Some or all of the struts 3061 may be attached, e.g. with sutures as described herein, to the body 3002 and/or the cover 3100 at one or more attachment locations.

Each recapture strut 3061 may include an inner curved portion 3062 connected to a distal end of the hub 3050, a middle straight portion 3064, and/or an outer curved portion 3066 (for clarity, only some of the portions 3062, 3064, 3066 are labelled in the figures). In the deployed configuration, the inner curved portion 3062 extends from the hub 3050 primarily in a distal direction and then curves to face more outwardly radially. The middle straight portion 3064 extends from the inner curved portion 3062 primarily radially but also slightly distally. The outer curved portion 3066 extends from the middle straight portion 3064 primarily in the radial direction and then curves toward the distal direction. The portions may have different shapes in the delivery configuration inside a delivery catheter. In the delivery configuration, the portions may extend primarily distally. The portions may then take the deployed configuration as described upon deployment from the delivery catheter. In some embodiments, the struts 3061 may include fewer or more than the portions 3062, 3064, 3066.

The device 3000 may include ten of the proximal recapture struts 3061. Such configuration may accompany a device 3000 having a foam body 3002 with an outer diameter of 27 mm in the free, unconstrained state. Such configuration may accompany a device 3000 having a foam body 3002 with an outer diameter of 35 mm in the free, unconstrained state. In some embodiments, the device 3000 may have from about two to about thirty, from about four to about twenty, from about six to about eighteen, from about eight to about sixteen, from about ten to about fourteen, or other numbers of struts 3061. In some embodiments, the device 3000 has twelve of the proximal recapture struts 3061, for example for the 35 mm diameter device.

In the deployed configuration, each strut 3061 may extend radially outward and distally at an angle to the axis. This angle, measured relative to a portion of the axis that extends distally from the device 3000, may be from about 60° to about 89.9°, from about 65° to about 88.5°, from about 70° to about 85°, from about 72.5° to about 82.5°, from about 750 to about 80°, or other angular amounts. This angle may be much smaller when the device 3000 is in the delivery catheter. The struts 3061 may bend or flex when transitioning between, or when positioned in, the delivery and expanded configurations. The struts 3061 may bend or flex at the inner curved portion 3062, the middle straight portion 3064, and/or the outer curved portion 3066.

The proximal end 3042 of the frame 3040, such as the proximal face 3060, may therefore have a conical shape in the expanded configuration. The conical proximal face 3060 may facilitate with recapture of the device 3000 back into the delivery catheter. For example, the orientation of the struts 3061 inclining distally and radially outward from the hub 3050 in the expanded configuration provides an advantageous conical shape to the proximal face 3008 such that distal advance of the delivery sheath over the device 3000 will bias the struts 3061 inward and cause the device 3000 to stow back toward the delivery configuration and size for retrieval within the catheter.

The proximal face 3060 foreshortens considerably upon expansion of the device 3000 relative to the delivery configuration. “Foreshortening” here refers to the difference in axial length of the proximal face 3060 between the reduced delivery configuration and the expanded configuration (expanded either freely or as implanted). This length may be measured axially from the distal or proximal end of the hub 3050 to the distal ends of the outer curved portions 3066 of the recapture struts 3061. The proximal face 3060 may foreshorten by 50%, 60%, 70%, 80%, 90% or more. The proximal face 3060 has significantly more foreshortening upon expansion than the tubular body 3080, the latter of which may be referred to as the “working length” or “landing zone.” The landing zone is further described with respect to the tubular body 3080 herein.

As shown, the struts 3061 are angularly spaced about the axis in even angular increments. That is, looking at the frame 3040 from the distal or proximal end, the angles between the struts may be equal. In some embodiment, the struts 3061 may not be evenly angularly spaced about the axis as described. The struts 3061 may or may not be symmetrically disposed about the axis or about a plane that includes the axis.

In some embodiments, portions of the frame 3040 may be at various distances from the proximal end of the foam body 3002, such as the proximal end wall having the proximal face 3008. As shown in FIG. 5D, there may be a gap of size Z in the axial direction between the proximal face 3060 of the frame 3040 and the inner surface 3012 of the proximal face 3008. The length of Z may be one, two, three, four, five, six, seven, eight, nine, ten, or more millimeters. The length of Z may vary depending on the radial distance at which it is measured. For instance, the length of Z may decrease, increase, or combinations thereof, as measured along the length of the strut 3061. In some embodiments, the length of Z may be zero at more or points along the length of the strut 3061. As shown in FIG. 5E, the proximal face 3060 or portions thereof may contact the proximal inner surface 3012 of the foam body 3002. The inner curved portion 3062, the straight portion 3064, and/or the outer curved portion 3066 may contact the proximal end wall such as the inner surface 3012 and/or other portions of the foam body 3002. The hub 3050 may compress the proximal face 3008 or proximal end wall of the foam body 3002 slightly in a proximal direction as shown. The proximal face 3008 may therefore have a smaller thickness in this compressed region as compared to other portions of the proximal face 3008, for example portions adjacent to this compressed portion. The hub 3050 may be located based on the axial location of connection of the anchors 3090, 3094 to the sidewall 3014, as described herein. In some embodiments, the hub 300 may not compress the foam body 3002 as shown. In some embodiments, the proximal face 3060 may extend radially outwardly as shown. For instance, the struts 3061, or portions thereof for instance the straight portions 3064, may extend radially outwardly perpendicularly or generally perpendicularly to the longitudinal axis of the device 3000. The proximal face 3060 may extend radially outwardly and incline in a distal direction, as described herein, or it may incline in a proximal direction. The device 3000 may have any of these features in the constrained, unconstrained and/or implanted configurations.

The frame 3040 includes a tubular body 3080. The body 3080 provides a mechanical base structure for the device 3000, as further described. The tubular body 3080 is attached to a distal end of the proximal face 3060 of the frame 3040. The tubular body 3080 extends to the distal end 3044 of the frame 3040. The tubular body 3080 is attached at a proximal end to the outer curved portions 3066 of the recapture struts 3061, as further described. The tubular body 3080 may be attached to other portions of the recapture struts 3061. The tubular body 3080 of the frame 3040 may be attached to the body 3002 and/or the cover 3100, e.g. with sutures as described herein, at one or more attachment locations, as further described. The tubular body 3080 may be located within the cavity 3028. In some embodiments, the tubular body 3080 may be located partially or entirely within the foam body 3002, e.g. within the sidewall 3014.

The tubular body 3080 includes a series of proximal struts 3082 and distal struts 3086 (for clarity, only some of the struts 3082, 3086 are labelled in the figures). The proximal struts 3082 and/or distal struts 3086 may have rectangular, circular or other shaped cross-sections. In some embodiments, the proximal struts 3082 and/or distal struts 3086 have a cross-section, e.g. rectangular, with a width that is greater than a thickness, or vice versa, such that the struts 3061 are stiffer in one direction compared to another direction. The struts 3061 may be less stiff in the direction of flexing or bending, for example to facilitate contraction and expansion of the device 3000 in the delivery and expanded configurations. Proximal ends of pairs of adjacent proximal struts 3082 join at proximal apexes 3084. Each proximal strut 3082 is connected at a respective proximal apex 3084 to a respective outer curved portion 3066 of one of the recapture struts 3061. Each distal end of the proximal struts 3082 connects to a distal end of an adjacent proximal strut 3082 and to proximal ends of two distal struts 3086 at an intermediate vertex 3087. Pairs of adjacent distal struts 3086 extend distally to join at a respective distal apex 3088. A repeating pattern 3089, shown as a diamond shape, may be formed by adjacent pairs of proximal struts 3082 and adjacent pairs of distal struts 3086. Some or all of the proximal struts 3082 and/or distal struts 3086 may be attached, e.g. with sutures as described herein, to the body 3002 and/or the cover 3100 at one or more attachment locations. Some or all of the proximal struts 3082 and/or distal struts 3086 may be located within the cavity 3028. In some embodiments, some or all of the proximal struts 3082 and/or distal struts 3086 may be located partially or entirely within the foam body 3002, e.g. within the sidewall 3014.

There are the same number of proximal apexes 3084 as distal apexes 3088. As shown, there are eleven proximal apexes 3084 and eleven distal apexes 3088. The number of proximal and distal apexes 3084, 3088 may each be at least seven, at least eight, at least nine, at least ten, at least eleven, at least twelve, at least thirteen, at least fourteen, at least fifteen, at least sixteen, or fewer or more apexes. In some embodiments, there may not be the same number of proximal apexes 3084 as distal apexes 3088. In some embodiments, there may be more than one row of the pattern, e.g. diamond pattern, formed by the proximal struts 3082 and distal struts 3086. There may be two, three, four or more rows of the pattern. Some or all of the proximal apexes 3084 and/or distal apexes 3088 may be attached, e.g. with sutures as described herein, to the body 3002 and/or the cover 3100 at one or more attachment locations.

The body 3080 may be tubular, e.g. cylindrical or generally cylindrical, in the expanded configuration. The tubular body 3080 may be cylindrical, rounded, segmented, polygonal, tube-like, other shapes, or combinations thereof, all of which are subsumed non-exhaustively under the category “tubular.” The tubular shape is formed by the proximal struts 3082 and distal struts 3086 in the expanded configuration. The tubular shape may also be formed by the outer curve portions 3066 of the recapture struts 3061 in the expanded configuration. The tubular shape may also be formed by the foam body 3002 exerting an outward radial force on the frame 3040. The frame 3040 may therefore have a proximal conical section and a cylindrical working length. In some embodiments, the body 3080 may be conical or frustoconical, for example where the distal end is wider than the proximal end or vice versa.

The tubular body 3080 may be referred to as a “landing zone,” as described. This landing zone may refer to the axial length of the body 3080, from a distal-most end to a proximal-most end at the transition to recapture struts 3061, in the expanded configuration. The landing zone may have an axial length as measured from the proximal apex 3084 to the distal apex 3088. The length of the landing zone may be 10 mm or about 10 mm. The landing zone may have a length from about 5 mm to about 15 mm, from about 6 mm to about 14 mm, from about 7 mm to about 13 mm, from about 8 mm to about 12 mm, from about 9 mm to about 11 mm, or other lengths. The tubular body 3080 may foreshorten slightly upon expansion of the device 3000 relative to the delivery configuration. The tubular body 3080 has significantly less foreshortening upon expansion than the length of the proximal face 3060. The tubular body 3080 may foreshorten by no more than about 5%, 10%, 15%, 20% or 30%.

The frame 3040 self-expands upon delivery from the sheath. The proximal face 3060 and the tubular body 3080 will self-expand. Upon expansion, the radially outward portions of the tubular body 3080 will contact and compress the foam body 3002 against tissue of the LAA wall. The tubular body 3080, for example the proximal struts 3082 and distal struts 3086, will contact the inner surface 3018 of the sidewall 3014 and press against the sidewall 3014 so that the outer surface 3016 of the sidewall 3014 contacts and compresses against the LAA wall.

When compressed against the LAA wall, the foam body 3002 provides a larger “footprint” than the skeletal frame 3040 components and forms a complete seal. Thus, the sidewall 3014 acts as a force dissipation layer, spreading radial force out from the struts 3082, 3086 of the frame 3040 over a larger area than just the area of the individual struts 3082, 3086 (e.g. a larger area than just the area of the radially outer surfaces of the struts 3082, 3086). The use of the foam material in the body 3002 and the thickness of that foam, such as 2.5 mm, provide advantages in this regard over devices with thinner and less resilient materials than foam. For example, thin fabrics or similar materials that are pressed against the LAA wall with a skeletal frame will not spread the radial force out, and may even sag or otherwise bend, creating gaps and an unsealed portion of the LAA wall. The foam body 3002 as described herein will take the shape of the LAA wall to create a complete circumferential seal and will also spread out the radial forces from the frame 3040 to create a stronger seal and retention with the foam body 3002.

Further, the device 3000 described herein with the compressible body 3002 allows for a structural frame 3040 that is compliant due to the smaller required radial force from the frame 3040. For example, existing devices with a non-compressible fabric material will have a less effective seal, and so the structural elements of those devices must provide larger radial forces to compensate and ensure an effective seal, resulting in a less compliant device. In contrast, the current device 3000 provides advantages in this regard by having the compressible foam body 3002, allowing for among other things smaller radial forces from, and thus better compliance of, the frame 3040, while still providing an effective seal. This structural configuration has a cascading effect in terms of performance advantages. For instance, the compliance of the device 3000 allows for delivery off-axis while still providing an effective seal, among other advantages as further described herein.

The frame 3040 includes a series of proximal anchors 3090. Each proximal anchor 3090 extends from a respective intermediate vertex 3087. The proximal anchors 3090 may extend from other portions of the tubular body 3080. As shown, in the deployed configuration, the proximal anchors 3090 extend from the tubular body 3080 radially and proximally. The proximal anchors 3090 may extend into an adjacent region of the sidewall 3014. The proximal anchors 3090 may extend through the outer surface 3016 of the sidewall 3014 to penetrate tissue adjacent the device 3000.

The frame 3040 includes a series of distal anchors 3094. Each distal anchor 3094 extends from a respective distal apex 3088. The distal anchors 3094 may extend from other portions of the tubular body 3080. As shown, in the deployed configuration, the distal anchors 3094 extend from the tubular body 3080 radially and proximally. The distal anchors 3094 may extend into an adjacent region of the sidewall 3014. The distal anchors 3094 may extend through the outer surface 3016 of the sidewall 3014 to penetrate tissue adjacent the device 3000. The anchors 3090, 3094 may incline radially outward in a proximal direction to engage the tissue to resist proximal movement of the device 3000.

The anchors 3090, 3094 are elongated structural members. The tips of the anchors 3090, 3094 may be sharpened to facilitate tissue engagement and penetration. The anchors 3090, 3094 may be straight, extending generally along a local axis thereof. The anchors 3090, 3094 may have a curved or other non-straight proximal portion where they attach to the tubular body 3080. In some embodiments, the anchors 3090, 3094 or portions thereof may be non-straight, curved, rounded, segmented, other trajectories, or combinations thereof. In some embodiments, the tissue engaging tips may be curved. In some embodiments, the anchors 3090, 3094 may have engagement features extending radially away from the anchor 3090, 3094, such as barbs, hooks, or other features.

The cross-section of the anchors 3090, 3094 may be rectangular. In some embodiments, the cross-section may be circular, rounded, non-rounded, square, rectangular, polygonal, other shapes, or combinations thereof. The cross-sections may or may not be uniform along the length of the anchor 3090, 3094. The anchors 3090, 3094 may be about 0.006″ thick and about 0.008″ wide. The anchors 3090, 3094 may range from about 0.003″ to about 0.009″ in thickness and from about 0.003″ to about 0.015″ in width. The cross-section of the anchors 3090, 3094 may reduce in size, for example taper, toward the distal tip.

In some embodiments, the anchors 3090, 3094 in the deployed configuration are inclined at an incline angle of about 300 relative to a portion of the central axis that extends proximally from the device 3000. This incline angle may be from about 10 degrees to about 50°, from about 15° to about 45°, from about 20° to about 40°, from about 25° to about 35°, or about 30°. This incline angle of the anchors 3090, 3094 in the delivery configuration may be smaller than in the deployed configuration. The deployed anchors 3090, 3094 may have the angle B.

The anchors 3090, 3094 may have various lengths. The length of the anchor 3090, 3094 is measured from a proximal end that connects to the tubular body 3080 to a distal tissue engaging tip of the anchor. In some embodiments, the length of the anchors 3090, 3094 may be from about 0.5 mm to about 10 mm, from about 1 mm to about 9 mm, from about 2 mm to about 8 mm, from about 3 mm to about 7 mm, from about 4 mm to about 6 mm, about 5 mm, or other greater or lesser lengths. In some embodiments, the anchors 3090, 3094 are 5 mm long. In some embodiments, the anchors 3090, 3094 are about 5 mm long. In some embodiments, the anchors 3090, 3094 have a length of at least 2.5 mm, at least 3 mm, at least 3.5 mm, at least 4 mm, at least 4.5 mm, at least 5 mm or more. The anchors 3090, 3094 may each be the same or similar length. In some embodiments, the anchors 3090, 3094 may not be the same length. In some embodiments, some or all of the proximal anchors 3090 may have lengths that are less than or greater than some or all of the lengths of the distal anchors 3094. The anchors 3090, 3094 may have the length L. Further, the outer tips of the deployed anchors 3090, 3094 may extend to an outer radial location that is less than, the same as, or more than a radially outermost surface of the foam body 3002.

In the expanded configuration, the anchors 3090, 3094 extend for a length outside of the uncompressed sidewall 3014. This length of the anchor 3090, 3094 is measured along a local longitudinal axis of the anchor from the outer surface 3016 of the body 3002 to the distal tip of the anchor. The anchors 3090, 3094 may extend through the sidewall 3014 and/or the cover 3100, and then be trimmed so that the anchors 3090, 3094 extend beyond the sidewall 3014 and/or cover 3100 by the desired length. In a free, unconstrained state, the anchors 3090, 3094 extend about 0.5 mm beyond the outer surface 3016 of the sidewall 3014. In some embodiments, in the free, unconstrained state, the anchors 3090, 3094 extend beyond the outer surface 3016 of the sidewall 3014 for a length of from about 0.1 mm to about 1.5 mm, from about 0.2 mm to about 1.25 mm, from about 0.3 mm to about 1.0 mm, from about 0.4 mm to about 0.8 mm, from about 5 mm to about 0.6 mm, or other greater or lesser lengths. In a compressed state, such as in the delivery configuration or after implantation, the anchors 3090, 3094 extend about 1.0 mm beyond the outer surface 3016 of the sidewall 3014. In some embodiments, in the compressed state, the anchors 3090, 3094 extend beyond the outer surface 3016 of the sidewall 3014 for a length of from about 0.25 mm to about 2.5 mm, from about 0.5 mm to about 2 mm, from about 0.75 mm to about 1.5 mm, from about 0.875 mm to 1.125 mm, or other greater or lesser lengths.

The geometry of the anchors 3090, 3094 provides several advantages. For example, the relatively long length allows for flexibility of the anchors 3090, 3094. This provides for potentially less trauma to the LAA tissue should the device 3000 need to be unanchored and/or retrieved. The anchors 3090, 3094 are less susceptible to loss of strength with off-axis orientation within the LAA. Further, the anchors 3090, 3094 provide high resistance to pull out. For instance, the device 3000 may provide at least about 0.5 lb-force of dislodgment resistance from the LAA. Such pullout tests may be simulated with in vitro or benchtop models, as further described below.

The anchors 3090, 3094 in the illustrated embodiment are located in two circumferential rows. One row is located proximal to the other distal row. Each row has ten anchors each. This configuration may be incorporated, for example, in the device 3000 having a foam body 3002 with a free, unconstrained outer diameter of 27 mm. Each row may have fourteen anchors each. This configuration may be incorporated, for example, in the device 3000 having a foam body 3002 with a free, unconstrained outer diameter of 35 mm. In some embodiments, a single row of anchors 3090, 3094 may have twelve anchors. In some embodiments, a single row of anchors 3090, 3094 may have from two to twenty-four, from four to twenty-two, from five to twenty, from six to eighteen, from seven to sixteen, from eight to fifteen, from nine to fourteen, from ten to thirteen anchors, or greater or fewer amounts of anchors 3090 or 3094. In some embodiments, there may only be one row or greater than two rows of anchors. The anchors 3090, 3094 may be spaced circumferentially in a single row. In some embodiments, the device has twenty-four total anchors 3090, 3094, with each row having twelve anchors, and twelve of the proximal recapture struts 3061, for example for the 35 mm diameter device 3000. In some embodiments, the device has twenty total anchors 3090, 3094, with each row having ten anchors, and ten of the proximal recapture struts 3061, for example for the 27 mm diameter device 3000.

In embodiments with multiple rows of anchors 3090, 3094, the rows may be circumferentially offset, as shown. That is, as viewed from the proximal or distal end of the device 3000, the anchors 3090, 3094 are angularly spaced apart from each other about the axis. The anchors 3090, 3094 may not be circumferentially offset, e.g. they may be evenly angularly spaced when viewed as described. The anchors 3090, 3094 are located axially at or near a middle portion of the sidewall 3014. The anchors 3090, 3094 may be located such that the tips of the anchors 3090, 3094 extend to adjacent tissue at a middle portion of the sidewall 3014. The offset and middle locations of the anchors 3090, 3094 may ensure engagement with the LAA tissue distal to the ostium. Having the anchors 3090, 3094 located at the largest width, increases the stability of the device 3000. With a cylindrical or generally cylindrical shaped device 3000, the anchors 3090, 3094 effectively sit on the largest diameter of the device 3000. The cylindrical shape provides advantages over typical LAA occluders which taper distally thus decreasing implant stability and locating the anchors on a smaller diameter than the ostial diameter of the occluding surface. In addition to adding stability, the cylindrical shape of the device 3000 along the axial length helps with dislodgement resistance by allowing the anchors 3090, 3094 to be placed on the largest diameter section of the device 3000. In some embodiments, the anchors 3090, 3094 may be located proximal, distal, or centrally along the length of the frame body 3080. In some embodiments, the anchors 3090, 3094 may not be offset and/or may not be angularly evenly spaced.

The anchors 3090, 3094 may provide advantageous flexibility, as demonstrated by pullout tests and in comparison to existing devices. For example, the device 3000 was tested to determine the force required to dislodge the device 3000 from a simulated tissue model by pulling the device 3000 proximally outward from the model. A low durometer silicone tube with a circular inner diameter (ID) was used as the model. For the device 3000 having a foam body 3002 with a 27 mm outer diameter in a free unconstrained state, tubes with ID's of 16.5 mm, 21 mm and 25 mm were tested. The pullout forces for existing devices drop off significantly going up to a 21 mm model, whereas the forces for the device 3000 drop only slightly.

In the largest diameter (25 mm) model, where there is not a lot of interference in the fit, the forces for the existing devices approach zero as the device does not engage the model wall because the anchors are sitting at a smaller diameter on a trailing edge of the device. The device 3000 consistently resists dislodgment with about 0.7 lbs of force. Since there is very little friction resisting pullout, that force is almost entirely resisted by the anchors 3090, 3094. When examining failure modes, all devices eventually begin to slide out of the model. Upon failure, the anchors 3090, 3094 fold backward or sideways before slipping starts. Assuming 0.7 lbs force is required to cause all twenty anchors 3090, 3094 to fold backward, then the force per anchors is estimated to be about 0.035 lbs.

The frame 3040 may be laser cut. The tubular body 3080 may be laser cut from a single tube. The body 3080 may be cut from a tube having a thickness from about 0.002″ to about 0.014″, or about 0.008″. The tube may have an outer diameter (OD) from about 0.05″ to about 0.30″. The tube may have an outer diameter (OD) of 0.124″ for the 27 mm device 3000 (i.e. the embodiment of the device 3000 having a foam body 3002 with an OD of 27 mm in the unconstrained, free state). The tube may have an OD of 0.163″ for the 35 mm device 3000 (i.e. the embodiment of the device 3000 having a foam body 3002 with an OD of 35 mm in the unconstrained, free state).

In some embodiments, the body 3080 is laser cut from a superelastic nitinol tube, however, numerous other biocompatible metallic materials can be utilized such as shape memory Nitinol, stainless steel, MP35N, or Elgiloy®. The frame 3040 is self-expandable. In some embodiments, a balloon-expandable frame 3040 could be utilized. Additionally, the body 3080 could be fabricated from drawn wire as opposed to being laser cut from a tube.

As shown, an embodiment of the device 3000 includes the frame 3040 having ten proximal recapture struts 3061 and twenty total anchors 3090, 3094, with the foam body 3002 having an outer diameter of 27 mm. In some embodiments, the device 3000 may include the frame 3040 having fourteen proximal recapture struts 3061 and twenty-eight total anchors 3090, 3094, with the foam body 3002 having an outer diameter of 35 mm.

In one embodiment, the frame 3040 includes a proximal hub 3050, tether pin 3051, front face with ten or fourteen recapture struts 3061, a diamond pattern cylindrical body 3080, and twenty or twenty-eight anchors 3090, 3094. The frame proximal face 3060 supports recapture, the frame body 3080 supports the foam cylinder body 3002, and the anchors 3090, 3094 located on the cylinder provide resistance to embolization.

The design of the device 3000 provides numerous advantages, some of which have been described. As further example, the frame 3040 provides many advantages, including but not limited to: 1) implant radial stiffness/compliance—the frame 3040 provides enhanced radial stiffness while still being sufficiently compliant to allow for off-axis implantation, recapture, etc.; 2) dislodgement resistance—the frame 3040 provides for high pullout strength, as described; 3) transcatheter delivery—the frame 3040 can be compressed into a delivery catheter and then fully expand when delivered; 4) recapture—the frame 3040 allows for recapture/retrieval into the delivery catheter after deployment or even after implantation in the LAA; and 5) mechanical integrity—the frame 3040 has acute and long term structural integrity, for example the ability to withstand loading into the delivery catheter, deployment from the catheter, and cyclic loading/fatigue. The frame 3040 also provides a conformable structure to enable the foam body 3002 to compress against the LAA tissue to facilitate sealing and anchoring with minimal compression (oversizing). The resulting compliance of the frame 3040 provides better anchoring than existing solutions, as described.

As further example, the device 3000 seals against irregularly shaped LAA ostia and necks. For instance, a combination of a Nitinol frame 3040 with a foam body 3002 having a coating of PTFE and cover 3100 of ePTFE contribute to ability of the device 3000 to conform to the anatomy and seal against irregular projections and shapes, while providing a smooth thromboresistent LA surface.

As further example, the device 3000 provides for controlled & safe delivery. The design of the combined frame 3040 and foam body 3002 facilitates delivery in a controlled fashion by slowing the speed of expansion. The bumper 3026 acts as an atraumatic leading edge portion when delivering the implant into the LAA mitigating the risk of injury. The user has the ability to recapture and redeploy the device 3000, if necessary. A flexible tether 3240 attachment, as further described, from the delivery catheter to the device 3000 permits the device 3000 to sit tension free immediately following implantation so the user can ensure final appropriate positioning prior to release of the device 3000.

As further example, the device 3000 provides for simplified placement. The foam-covered cylindrical design makes alignment of the device 3000 with the central axis of the LAA during delivery non-critical (by allowing deployment up to, for example, 45 degrees off-axis), which is designed to simplify the implantation procedure, as further described.

As further example, the device 3000 provides for simple sizing. The foam and frame design contributes to the ability to need only two diameters (e.g., 27 mm and 35 mm) to seal the range of expected LAA configurations and diameters (e.g. targeting LAA diameters of 16 to 33 mm). The conformability of the foam and frame allow the 20 mm long implant to fit into LAA's as short as 10 mm deep. The short landing zone requirement (LAA depth) of the device 3000, combined with the need for only two implant diameters, enables treatment of a wide range of LAA anatomies with minimal need for burdensome echo and CT sizing. The conforming nature of the implant is key to facilitating a simple to use product platform that is adaptable to a variety of anatomic structures.

As further example, the device 3000 provides thromboresistant materials and design. The removable tether leaves a smooth, metal-free surface in the LA. Thromboresistant materials (PTFE-coated foam and an ePTFE cover) create a smooth LA face (no metal attachment connection) to reduce anticoagulation needs, enhance thromboresistance, and encourage endothelialization.

As further example, the device 3000 provides thin, low profile anchors 3090, 3094 around the midpoint of the device 3000 to provide secure yet atraumatic anchoring.

The foam body 3002 has a distal bumper 3026, for example as shown in FIG. 4C. The bumper 3026 may be a foam distal region of the body 3002, such as a distal portion of the sidewall 3014. The bumper 3026 may be a portion of the foam body 3002 that extends beyond the distal end 3044 of the frame 3040. The bumper 3026 may extend beyond the distal end 3044 of the frame 3040 in the delivery configuration and in the deployed configuration. The body 3002 may be attached to the frame 3040 in various locations such that the body 3002 may stretch in some embodiments, for example in the delivery configuration, to ensure the bumper 3026 extends beyond the frame 3040 upon initially retracting the sheath during delivery.

The device 3000 can conform both in length and diameter due to conformability of both the foam body 3002 and the frame 3040. This allows for the device 3000 to accommodate most patient LAA anatomies with only a couple or few different sizes of the device 3000, such as 27 mm and 35 mm outer diameter body 3002 as described herein, and one length, such as 20 mm. The frame 3040 may thus be shorter than the foam body 3002, resulting in some embodiments in about 5 mm of foam bumper 3026 distal to the distal-most end of the frame 3040. The distal bumper 3026 acts as an atraumatic tip during delivery of the device 3000 and can be compressed following implantation to allow the device 3000 to conform to appendages with a depth (landing zone) as short as 10 mm. This ability to conform both in length and diameter is due to the conformability of both the foam body 3002 and the frame 3040.

The length of the bumper 3026 may be measured axially from the distal-most end of the frame 3040 to the distal surface 3022 of the body 3002. For example, the bumper 3026 may extend from the distal apexes 3088 to the distal surface 3022. The bumper 3026 may have a length of 5 mm or about 5 mm. The bumper 3026 may have a length of about 1 mm, 2 mm, 3 mm, 4 mm, 5 mm, 6 mm, 7 mm, 8 mm, 9 mm or more. The bumper 3026 may have a length from about 2.5 mm to about 7.5 mm, from about 3 mm to about 7 mm, from about 3.5 mm to about 6.5 mm, from about 4 mm to about 6 mm, from about 4.5 mm to about 5.5 mm.

In some embodiments, the bumper 3026 may fold in response to axial and/or radial compression of the device 3000. The bumper 3026 may fold inward, for example radially inward. The folds may be in the axial or approximately in the axial direction. The folds may be circumferential or approximately in the circumferential direction. The folds may be combinations of the radial and circumferential directions, or angled with respect thereto. The folding of the bumper 3026 is further discussed herein, for example in the section “Device Compliance.”

FIGS. 8A-8C are proximal perspective views of the frame 3040 having a cap 3180. FIG. 8D is a distal perspective view of the cap 3180. In some embodiments, the pin 3051 is placed across the proximal hub 3050 diameter and serves to engage the delivery catheter tether 3240 (e.g. a suture), which is wrapped around the pin 3051 for temporary attachment to the delivery catheter 3220, as described further herein for example with respect to FIGS. 7A-7B. As shown, the hub 3050 has a pair of opposite side openings 3053 extending through a sidewall of the hub 3050. The cap 3180 has a corresponding pair of opposite side openings 3190 extending through a sidewall 3184 of the cap 3180. When the cap 3180 is assembled with the hub 3050, the pin 3051 may be inserted through the aligned pairs of openings 3053, 3182. The assembly can be further secured by welding the ends of the pin 3051 to the hub 3050.

As shown in FIG. 8D, the cap 3180 includes a proximal end 3182 and a distal end 3184. The cap 3180 includes a rounded sidewall 3186 extending from the proximal end 3182 to the distal end 3184. The sidewall 3186 defines a longitudinal opening 3188 through the cap 3180. The sidewall 3186 includes a pair of lateral openings 3190 located opposite each other. The cap 3180 includes a flange 3192 at the proximal end 3182 extending radially outward.

The cap 3180 is formed from titanium and the pin 3051 is formed from Nitinol or superelastic Nitinol. In some embodiments, the cap 3180 and/or pin 3051 may be formed from other materials, for example numerous biocompatible metallic or polymeric materials such as shape memory Nitinol, stainless steel, MP35N, Elgiloy, polycarbonate, polysulfone, polyether ether keytone (PEEK), or polymethyl methylacrylate (PMMA) or other materials.

The cap 3180 and pin 3051 facilitate attachment to the tether 3240. The cap 3180 and pin 3051 also mitigate damage to the foam body 3002 during recapture of the device 3000. The cap 3180 also creates an atraumatic surface for the hub 3050 of the frame 3040. For example, the cap 3180 may prevent the hub 3050 from cutting through the foam body 3002 as the device 3000 is collapsed into an access sheath. Without the cap 3180, the sharp edges of the hub 3050 may shear through the foam body 3002 during recapture of the device 3000 into the access sheath.

The various embodiments of the LAA devices shown and described with respect to FIGS. 1-8D may be used in the surgical devices and methods described herein, such as those shown and described with respect to FIGS. 9A-61.

### C. Surgical Clip and Delivery System

Various embodiments of LAA occlusion devices may be used in various surgical approaches, as further described. A surgical clip embodiment is described in this section with respect to FIGS. 9A-34. Other embodiments for occluding the LAA of the heart are described in following sections and with respect to FIGS. 35-61. For the surgical approaches, the LAA can be entered externally from what would be considered the distal end of the LAA during an interventional closure procedure (e.g. the tip of the LAA), etc. as described above.

In some embodiments, a Surgical Left Atrial Appendage Closure (SLAAC) clip 200 or device may be implemented to close the LAA. SLAAC-clips and similar devices may be used surgically via a thoracoscopic approach or during open heart surgery.

The SLAAC-clip 200 can be placed within the left atrium under transesophageal echocardiography (TEE) guidance. In some embodiments, the SLAAC-clip 200 is a multi-leg metal clip with distal anchors that can be attached to a delivery catheter which is placed though a trocar. When the clip is placed within the LAA, the clip can be opened and the trocar withdrawn. As the trocar is withdrawn, the anchors embed into the tissue of the LAA and close off the ostium. In some embodiments, the SLAAC-clip can be further anchored in place with a suture tether that can be fixed to the exterior wall of the LAA. This external fixation device can also seal the entry site. The clip can be fabricated from 2 to 10 anchors or fronds. It is expanded within the LAA. To place the clip into a closed configuration after engagement with the ostial tissue, a collar is moved up the frond and over a locking bump.

FIGS. 9A and 9B depict a delivery system 300 and an SLAAC-clip 200, which can be loaded within the delivery system 300. When the SLAAC-clip 200 is loaded within the delivery system 300, the combination may be referred to as a combined SLAAC-clip/delivery system 101. The delivery system 300 can be used for delivering the SLAAC-clip 200 through the tip of the LAA.

As depicted in FIG. 9A, the SLAAC-clip 200 may be configured to fit within the delivery system 300 for deployment at the delivery site where the SLAAC-clip 200 is pushed out of the distal end of the delivery system 300. After the SLAAC-clip 200 is positioned in the LAA, the empty delivery system is removed. In some embodiments, the delivery system is removed by disassembling some of the components. FIG. 9B depicts the SLAAC-clip 200 outside of and detached from the delivery system after release.

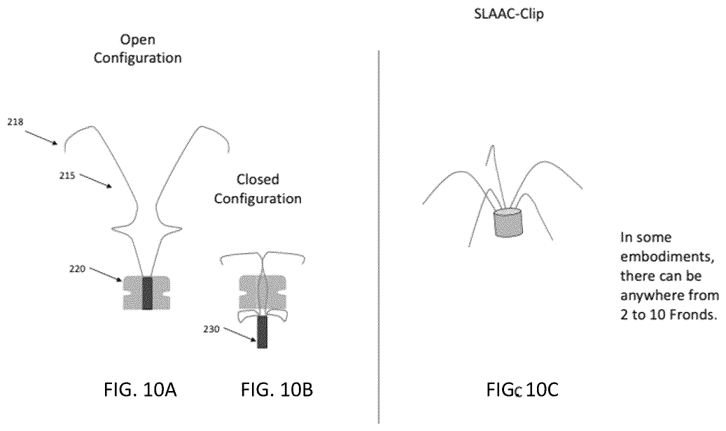

As shown in FIG. 10A-10C, the SLAAC-clip 200 can include one or more fronds 215, anchors 218, a collar 220, and/or a pin 230. The SLAAC-clip 200 may have various configurations to fit the needs of the implantation process. For example, FIG. 10A depicts an open configuration, and FIG. 10B depicts a closed configuration. The collar 220 may have a radial notch around the perimeter for coupling to the delivery system. The pin 230 may be positioned in a channel within the middle of the collar 220 in the open configuration. The fronds 215 may extend from the pin 230. For example, FIG. 10A depicts the SLAAC-clip 200 in an open configuration with two fronds fully extended from the collar and pin. The ends of the fronds 215 may curve into anchors for hooking into the tissue at the LAA. FIG. 10B depicts the SLAAC-clip 200 in a closed configuration with the collar 220 advanced relative to the pin 230. Upon deployment of the SLAAC-clip 200, the anchors may be embedded in the LAA ostium in an open position. As the SLAAC-clip 200 transitions to the closed configuration, the anchors may be brought together with the ostium, closing off the LAA.

In some embodiments, such as depicted in FIG. 10C, the SLAAC-clip 200 may have more than two fronds 215. There may be two, three, four, five, six, seven, eight, nine, ten or more fronds 215 extending from the collar 220. Fronds 215 may extend radially and equidistance apart from the perimeter of the collar 220. More fronds 215 may increase the number of sites that the SLAAC-clip 200 embeds into the ostium.

FIGS. 11A-11G depict the components of the delivery system 300. FIG. 11A is an internal view of the delivery system 300. The delivery system may have an internal tube 1102 running longitudinally to the device. Fronds 215 and pin 230 of the SLAAC-clip 200 may be constrained within the inner tube of the delivery system. FIG. 11B is a side view of the delivery system showing an outer tube 330 and pincers 340. The pincers 340 may run along the length of the outer tube 330 with angulated distal ends. The angulated distal ends can couple to the collar 220 at the radial notch 1104. Squeeze points 1106 may be positioned at the proximal ends of the pincers 340. When the squeeze points are depressed, the hooks at the distal ends move apart. FIG. 11C depicts a side view of the delivery system when the pincers are depressed which may release the collar 220 and SLAAC-clip 200. Pincers 340 may be coupled to the delivery device 300 via multiple methods. As depicted, each pincer may extend through slits 324 in the outer tube 330, depicted in FIG. 11D, to a fulcrum point allowing the pincer to open and close when depressed. The fulcrum point may be attached in several ways, such as elastically, a pivot pin, interlocking parts, or the like.

In another embodiment, the pincers 340 may open via a collet-like mechanism where the pincers 340 are naturally spring loaded outward and another tube is slid over them. The additional tube allows the pincers to be gathered together.

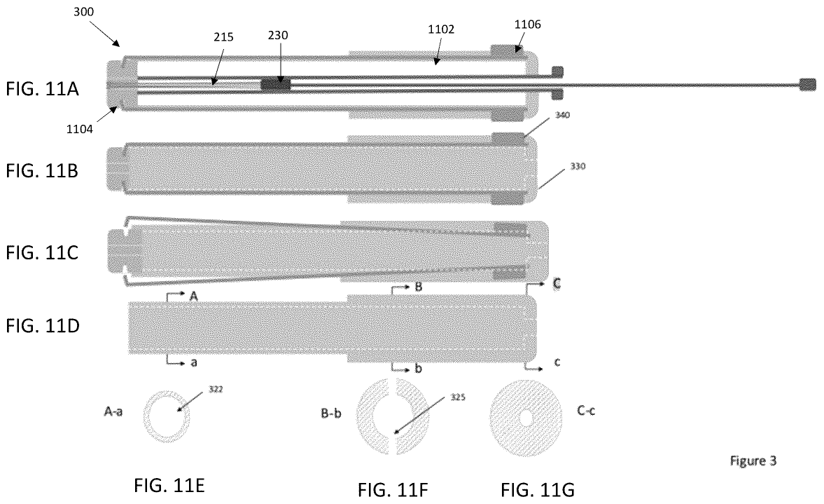

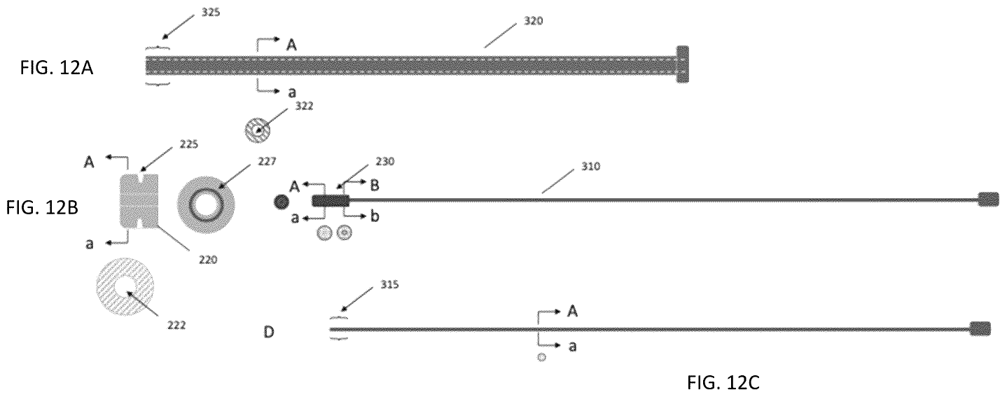

FIG. 11D depicts a side view of the outer tube 330. A central lumen 322 may run along the longitudinal axis of the outer tube 330. The outer tube 330 may have a distal region, a middle region, and a proximal region. Each of the regions of the outer tube may have varying thicknesses and components. For example, FIG. 11E illustrates a cross-section of the distal region of the outer tube 330 having a first thickness. FIG. 11F illustrates a cross section of the middle region which may have slits to accommodate the pincers 340. FIG. 11G depicts a cross section of the proximal region where the thickness of the outer tube 330 is greater than that depicted in FIG. 11E. When the outer tube 330 has an increased thickness, the central lumen 322 may have a reduced diameter to accommodate an inner tube 320 as described with respect to FIGS. 12A-12C.

As depicted in FIGS. 12A-12C, inner components of the delivery system can couple together for deploying the SLAAC-clip 200. FIG. 12A depicts the inner tube 320 of the delivery system 300. The inner tube 320 may be a simple tube with a central lumen 321 running longitudinally. At the distal end, the inner tube 320 may have a threaded outer contour 325. FIG. 12B depicts several individual components of the delivery system. A side view of the collar 220 is shown with a central lumen. The central lumen 222 of the collar 220 may be in continuity with the central lumen of the inner tube when paired together. As previously discussed, the collar 220 may have a radial notch 225 around the outer perimeter to engage the pinchers 340 of the delivery system. The collar 220 may also have a threaded central lumen 227. The threaded outer contour 325 of the inner tube 320 may engage with the threaded central lumen 227 of the collar 220.

The delivery system 300 may also contain a push rod 310. A threaded element 315 may be at the distal end of the push rod 310. The threaded element 315 may engage with a threaded central lumen within the pin 230. In some embodiments, the pin 230 may be cylindrical in shape. The pin 230 may be designed as to prevent the pin 230 from extending beyond the collar 220 as it is pushed from inside the delivery system. In an example embodiment, the pin 230 may be stepped in diameter with a wider proximal end to prevent it from passing completely through the collar 220.

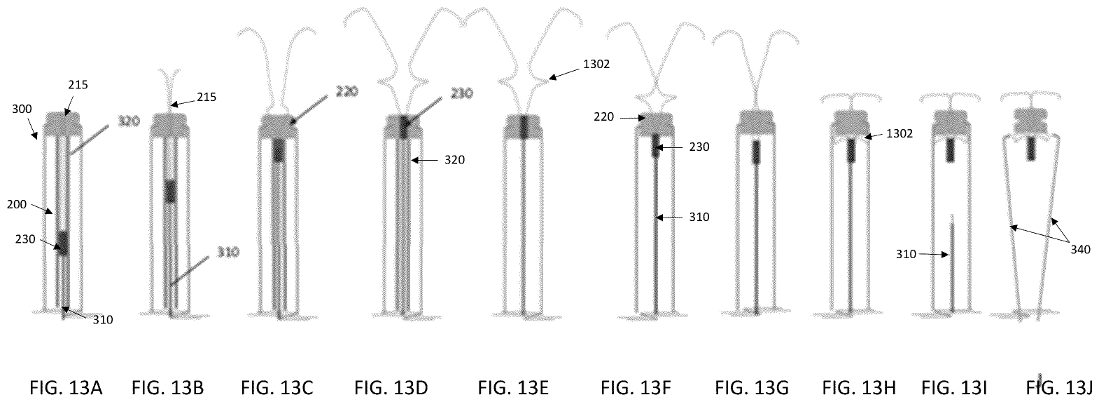

FIGS. 13A-13J depict various configurations of the delivery system 300 as it deploys the SLAAC-clip 200. The delivery system 300 as depicted may be used, either alone or in combination, with any of the delivery system components described herein. In FIG. 13A, the SLAAC-clip 200 and delivery system 300 are in an initial configuration that may be provided to the operator/user. The fronds 215 of the SLAAC-clip 200 are pictured fully within the delivery system and constrained by the inner tube 320. The push rod 310 and the connected pin 230 are retracted. FIG. 13B depicts the fronds 215 being expelled from the collar 220 at the distal end of the delivery system. The fronds may be expelled as the push rod 310 is advanced into the inner tube towards the distal end. As the pin 230 is pushed further into the delivery device, the fronds extend further from the distal end, as pictured in FIG. 13C. The push rod 310 can be advanced until the leading edge of the pin 230 meets the distal tip of the collar 220 through the central lumen, as pictured in FIG. 13D. The inner tube 320 may then be disengaged form the collar 220 by rotating to release from the threads as previously discussed. The delivery system with the inner tube 320 disengaged and removed is depicted in FIG. 13E. The fronds may be embedded into the tissue of the ostium, and the operator may close the fronds by retracting the pin 230 back through the collar 220. As depicted in FIG. 13F, the push rod 310 can be retracted back through the proximal end of the delivery system. As a result, the pin 230 is also pulled back through the proximal end of the collar 220. Retraction of the push rod 310 and pin 230 may cause the fronds to retract back through the collar 220 within the delivery system, as shown in FIGS. 13G and 13H. As the fronds 215, are withdrawn proximal to the proximal end of the collar 220, the fronds 215 can lock in place by allowing the proximal ends of the fronds to take on their unconstrained configuration within the delivery device, which can occur in the absence of the inner tube 320. In the unconstrained configuration, the proximal portion of the fronds may form a locking bump 1302 or locking barrier that prevents further distal movement of the fronds into the collar 220. The anchor portion of the fronds may be drawn together, pulling together any tissue in which they are embedded. An operator may determine if the fronds have been retracted sufficiently so that they are locked in place and/or that the clip is in a desired position by a number of various visualization tools and techniques, such as radiography, echography, guiding markings on the delivery system, direct visualization, and the like. Once the operator is satisfied with the position of the clip and the fronds, the push rod 310 may be released by unscrewing from the threading element at the pin 230, as depicted in FIG. 1. After the push rod 310 is retracted from the delivery system, the outer tube is removed by squeezing the pincers 340. FIG. 13J shows the pincers disengaging from the radial notch of the collar and the delivery system being removed from the SLAAC-clip 200.

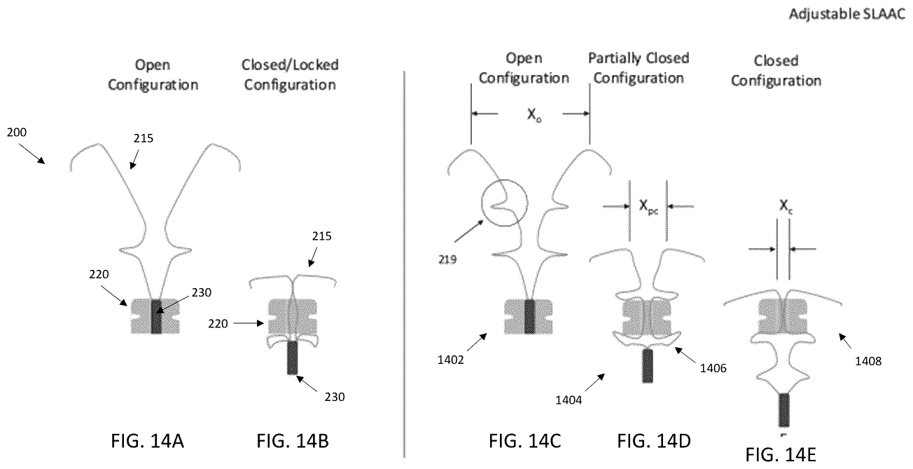

As shown in FIGS. 14A and 14B, the SLAAC-clip 200 may have an open configuration and a closed/locked position. When the SLAAC-clip 200 is in the open configuration in FIG. 14A, the fronds 215 are unconstrained by the inner tube or delivery device. The closed/locked position in FIG. 14B occurs when the pin 230 in retracted with the fronds through the collar 220, and the fronds 215 are able to assume their unconstrained configuration (e.g., by fold) at the proximal end of the collar.

In some embodiments, like pictured in FIGS. 14C-14E, the SLAAC-clip 200 may have an additional bump feature 219 in each of the fronds. FIG. 14C depicts the SLAAC-clip 200 in the open configuration 1402 with another bend in the fronds which may allow for additional configurations of the SLAAC-clip 200. When in an open configuration, distance X₀ is measured between an apex on each frond anchor. FIG. 14D depicts a partially closed configuration 1404 of the SLAAC-clip 200 where the fronds are pulled back through the collar up to the bump feature 1406. The distance between apexes on the anchors of the fronds is now X_{PC} in the partially closed configuration. Drawing the fronds and the bump feature completely through the collar 220 may result in a fourth configuration, the fully closed configuration 1408 as in FIG. 14E. The distance between apexes of the fronds is now X_{C}. Additional configurations such as the partially closed and fully closed may accommodate variability in the size of the LAA opening and thickness of tissue across patients.

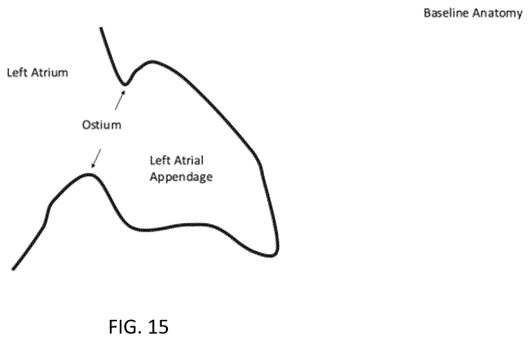

FIGS. 15-34 depict various features that may be used, either alone or in combination, with any of the LAA occlusion devices and methods described herein. In particular, FIGS. 15-34 show sequential views of an embodiment of a process for delivering and implanting the SLAAC-clip 200 via the delivery system.

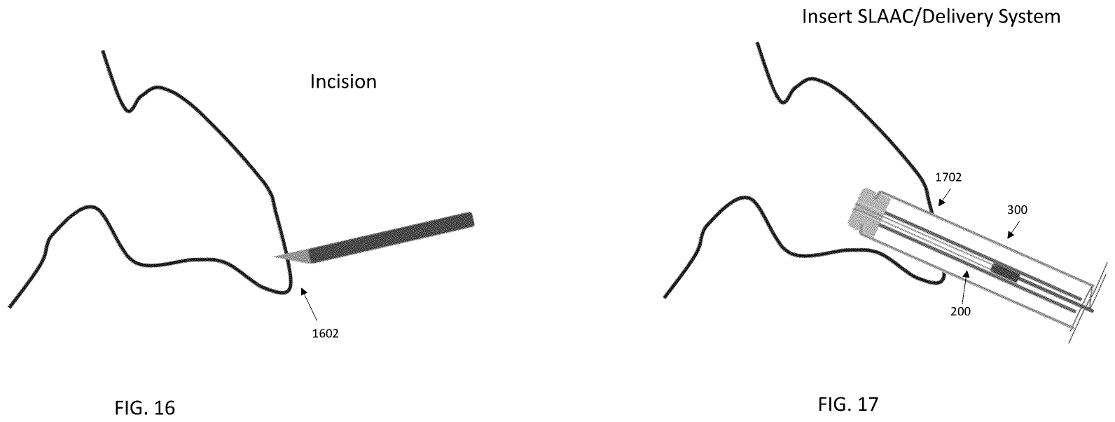

As shown in FIG. 16, the procedure for inserting the SLAAC-clip is initiated with the surgeon making an incision in the tail of the LAA 1602. The size of the incision may be large enough to accommodate the outer tube of the delivery system.

FIG. 17 depicts the delivery system 300 being inserted through the incision 1702 and positioned with the distal end in the LAA. An internal view of the delivery system shows that it is loaded with the SLAAC-clip 200 in the constrained position with the fronds in the inner tube. The outer tube is positioned far enough into the incision site and the LAA so that the SLAAC-clip deploys into the ostium.

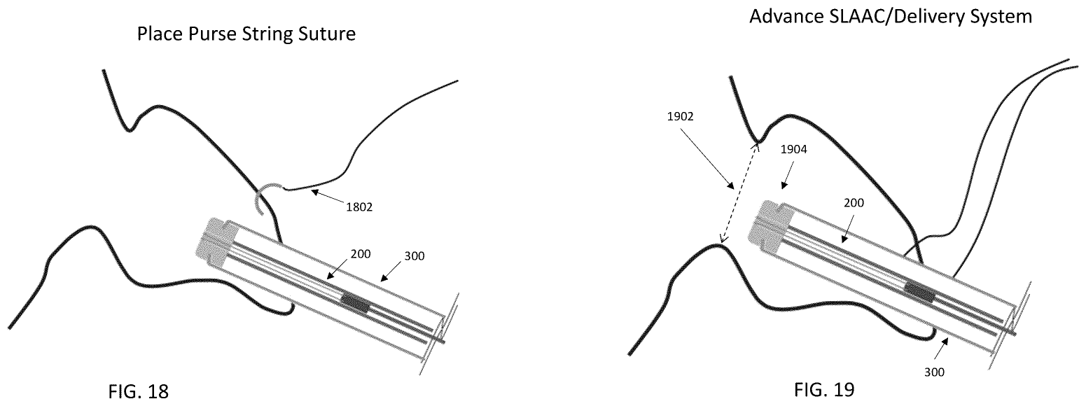

As depicted in FIG. 18, a purse string suture 1802 may be placed around the SLAAC-clip 200 and delivery system 300. The purse string suture 1802 may provide the option of sealing the tissue around the delivery system 300 to prevent excessive bleeding during the procedure. Additionally, including a purse string suture 1802 can help close the incision when the procedure is complete.

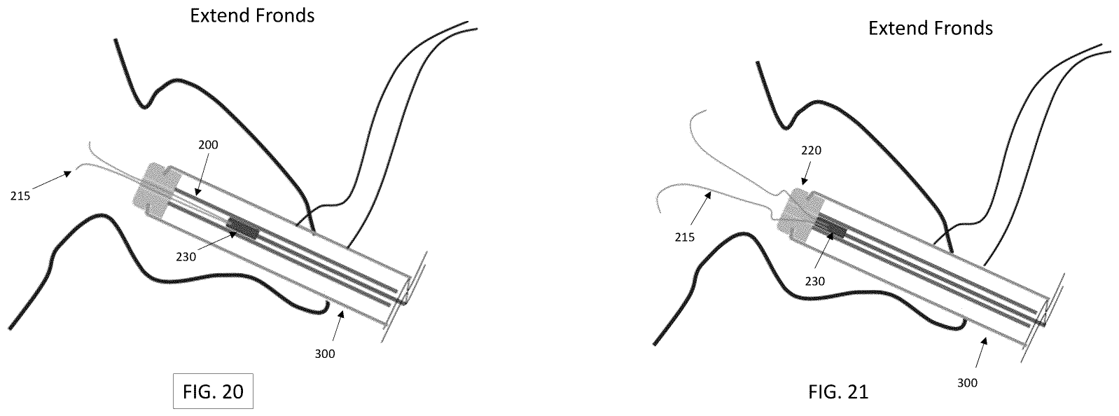

FIG. 19 depicts the delivery system 300 advancing towards the ostium 1902 to position the distal end 1904 for deployment of the SLAAC-clip 200. FIG. 20 depicts the delivery device 300 as the clip 200 is deployed within the ostium. Once the operator positions the delivery device, the proximal end of the push rod (not pictured) is pushed into the delivery system as the fronds 215 of the SLAAC-clip 200 begin to emerge out the distal end 1904 into the ostium.

FIG. 21 depicts the fronds 215 extending through the collar 220 in the ostium. The push rod has been inserted just enough so that the pin 230 has reached the collar 220. As the fronds 215 advance through the collar 220, they extend away from the delivery system 300.

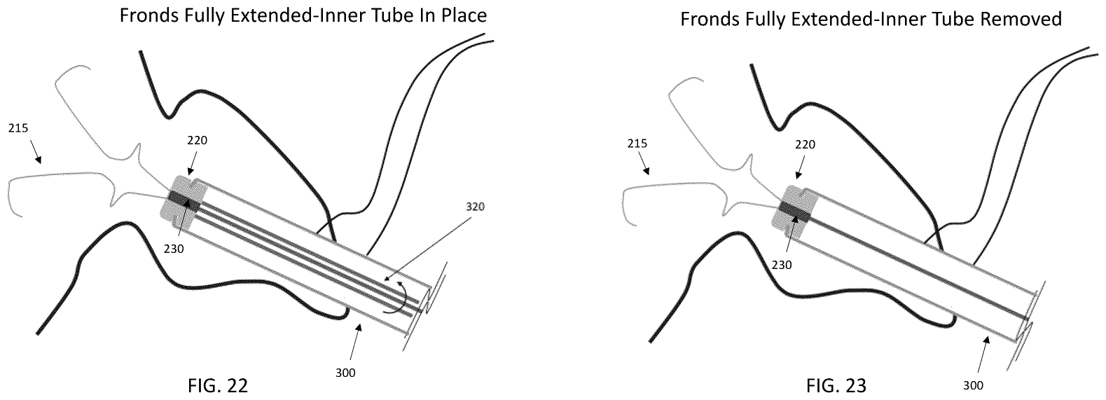

As pictured in FIG. 22, the SLAAC-clip 200 is in the open configuration once the push rod has been full inserted. The fronds 215 are extended from the collar 220 and the inner tube 2202 can be removed. As previously disclosed, the inner tube 320 is rotated to disengage the threading element (not pictured) with the collar 220. Once decoupled, the inner tube 320 can be removed from the proximal end of the delivery system 300. The pin 230 may be fully inserted into the center of the collar at this point in the deployment process.

FIGS. 23-26 depict the delivery system 300 being retracted from the LAA through the suture site with the fronds 215 fully extended. In some embodiments, positioning of inner components within the delivery system 300 does not change as the entire device is retracted back through the suture site of the LAA. Due to the removal of the inner tube, there is now space within the delivery device 300 for the fronds 215 to transition to an unconstrained configuration if retracted proximally into the outer tube 330. The delivery system 300 may be slowly removed from the LAA through the incision site.

FIG. 27 illustrates the anchors of the fronds 215 engaging with tissue of the ostium upon retraction of the delivery system 300. The open configuration of the SLAAC-clip allows the fronds 215 to engage with the tissue as the delivery system is retracted. In some embodiments, the delivery system 300 may be angled upon retraction from the LAA to ensure that the fronds 215 engage with the tissue in the desired location. Once the fronds 215 are engaged with the tissue of the ostium, the push rod 310 may be retracted through the proximal end of the outer tube. The pin coupled to the push rod 310 may be pulled back out of the collar 220 as the outer tube 330 remains is held in place.

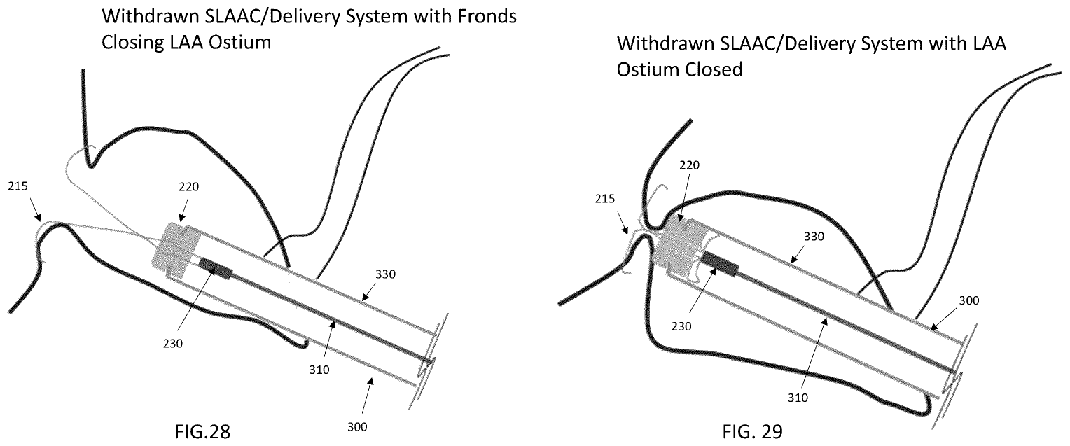

As shown in FIG. 28, the push rod 310 may be retracted manually away from the LAA. In some embodiments, the push rod 310 may be retracted so that the pin 230 is pulled closer to the proximal end of the outer tube 330. As the pin 230 is pulled inside the outer tube 330, the fronds 215 may also be pulled into the delivery system 300. The length of the fronds 215 outside the delivery system may shorten and pull the ostium together and closed.

The fronds 215 may be pulled tight into the delivery system 300 as the collar 220 advances toward the ostium, as depicted in FIG. 29. In one embodiment, the push rod 310 can be retracted until the fronds 215 are pulled tight against the ostium. As the push rod 310 is pulled, the collar 220 and outer tube 330 of the delivery system can be advanced towards the now closed ostium. The portion of the fronds 215 pulled inside the delivery system 300 can transition to the unconstrained configuration. The SLAAC-clip may collapse inside the delivery system 300 due to the lack of inner tube. When the fronds 215 are pulled tight, the clip may be in a locked position indicating that the ostium has been closed.

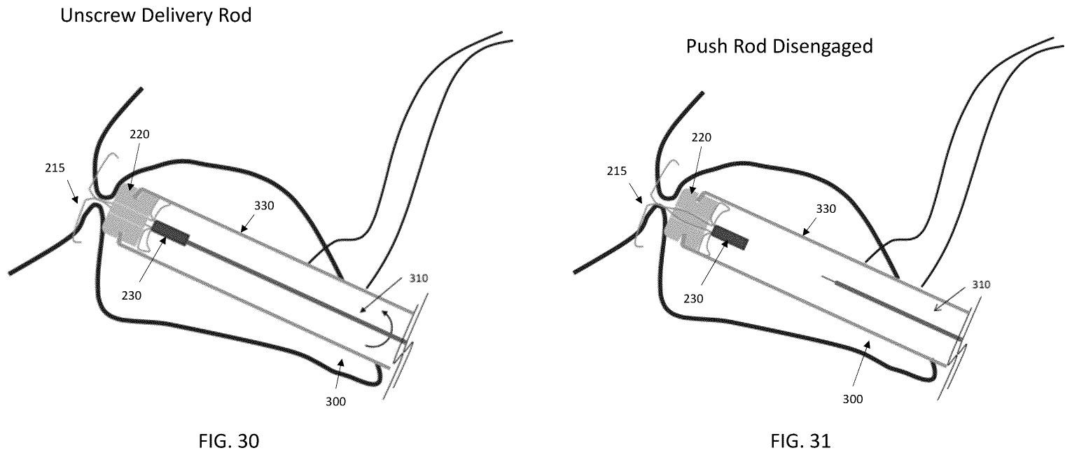

In some embodiments, after the SLAAC-clip has been pulled into the locked, unconstrained configuration, the delivery system 300 may be removed, as depicted in FIGS. 30-32. As FIG. 30 illustrates, once the ostium has been pulled closed, the operator may remove the push rod 310 from the delivery system. In some embodiments, the push rod 310 is rotated to uncouple the distal end from the thread element of the pin 230.

FIG. 31 depicts the push rod 310 completely uncoupled from the pin 230. In some embodiments, once unscrewed, the push rod 310 may be removed from the proximal end of the outer tube.

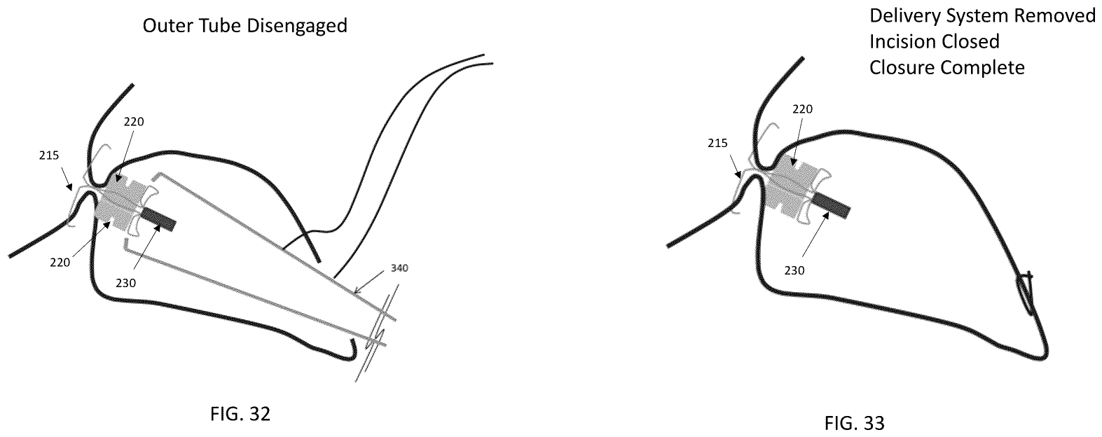

As shown in FIG. 32, the remaining components of the delivery system 300 may be removed from the LAA. In some embodiments, the operator may squeeze the pincers 340 so the distal ends of the pincer arms disengage from the radial notch 3202 of the collar 220. The pincers and outer tube 340 can be fully released from the SLAAC-clip. The outer tube 340 and attached pincers can be removed from the incision site of the LAA. The SLAAC-clip with the attached collar 220 and pin 230 remain embedded in the ostium tissue.

After removal of the delivery system from the LAA, the incision site may be closed as shown in FIG. 33. The ostium remains closed together due to the deployment of the SLAAC-clip.

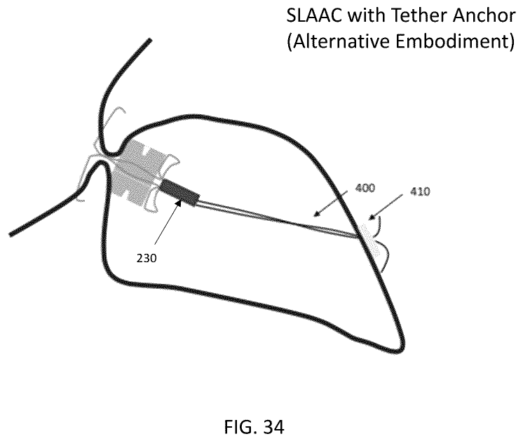

In some embodiments, as pictured in FIG. 34, a tether 400 may connect the pin 230 to the LAA wall. For example, the SLAAC-clip may be loaded into the delivery system 300 with a tether 400 connected to the proximal end of the pin 230. When the SLAAC-clip is deployed and the delivery system is removed from the LAA, the tether 400 may feed through the incision site to the outside of the LAA. In some embodiments, a pledgeted suture can be used to secure the tether to the outer LAA wall. The tether 400 helps keep the SLAAC-clip 200 and the fronds 215 in place in the ostium. In some embodiments, the externally fixated tether 400 can seal the incision site.

### D. Purse String/Lasso

FIGS. 35-43 depict sequential views of an LAA and delivery system for a delivery method for percutaneously delivering an LAA occlusion implant (such as the device 3000) through a transseptal puncture approach. Generally, the method can include a puncture or slit 3502 made in the back of the LAA 3504. A trocar 3508 containing the implant 3506 and delivery catheter can be positioned through the access site 3502. Once positioned inside the LAA, TEE imaging can be used to precisely orient the trocar at the ostium of the LAA. The implant 3506 can be deployed with a suture tether 3510 for attachment around the internal frame. The tether 3510 can work like a lasso in facilitating both repositioning and recapture of the implant. For example, in a situation where the implant 3506 needs to be collapsed, a pusher 3512 can be advanced from the delivery system to the tether 3510. The tether 3510 can be retracted to collapse the implant 3506. Once positioning of the implant 3506 is corrected, the trocar and delivery catheter can be retracted and the suture tether 3510 can be removed.

FIGS. 35-43 depict various features that may be used, either alone or in combination, with any of the LAA occlusion devices and methods described herein. In particular, FIGS. 35-43 show sequential views of an embodiment of a process for delivering an implant 3506 using a purse string or lasso suture 3510 for repositioning or retrieval. The device used for the purse string/lasso approach described with respect to FIGS. 35-43 may include any features of the devices shown and described with respect to FIGS. 3A-8D.

As depicted in FIG. 35, the procedure for inserting the implant 3506 with the purse string 3510 may initiate with the surgeon making an incision 3502 in the back of the LAA 3504. The size of the incision 3502 may be large enough to accommodate the trocar 3508. In some embodiments, the incision site is on the side of the LAA. In an alternative embodiment, the incision site is at the tip of the LAA.

FIG. 36 shows the trocar loaded with the implant 3506 near the incision in the LAA wall. The implant may be compressed within the delivery system 3508 and may expand upon deployment into the LAA.

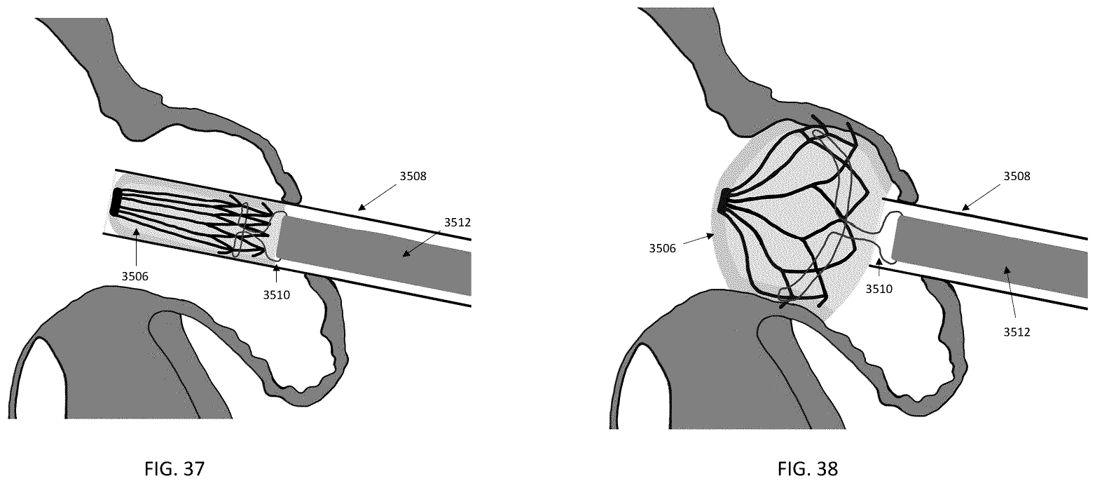

FIG. 37 shows the distal end of the trocar 3508 entering the LAA through the incision site. In some embodiments, the trocar 3508 is inserted into the LAA until the opening at the distal end of the trocar 3508 is at the ostium of the LAA. Various imaging techniques may be used to place the trocar 3508. For example, radiography, echography, guided markings on the delivery system, directed visualization, or a combination of any may be used by the operator.

As depicted in FIG. 38, when the trocar 3508 is properly positioned, the implant 3506 may be deployed into the LAA through the distal end of the trocar 3508. A suture 3510 attachment, as previously discussed, may facilitate repositioning and retrieval of the implant 3506 if required. In some embodiments, the suture 3510 may be connected to the frame of the implant 3506. For example, the suture 3510 may weave in and out of the frame diamonds as depicted in FIG. 38. Alternatively, eyelets could be added to the frame of the implant 3506 to accommodate the suture 3510 passing through.

After deployed from the trocar 3508, the implant 3506 may expand and fill the space between tissue in the ostium. The trocar 3508 may remain proximal to the deployed implant 3506 with the suture 3510 attached to the pusher 3512 within the delivery system. The trocar 3508 may still be positioned fully within the incision site of the LAA.

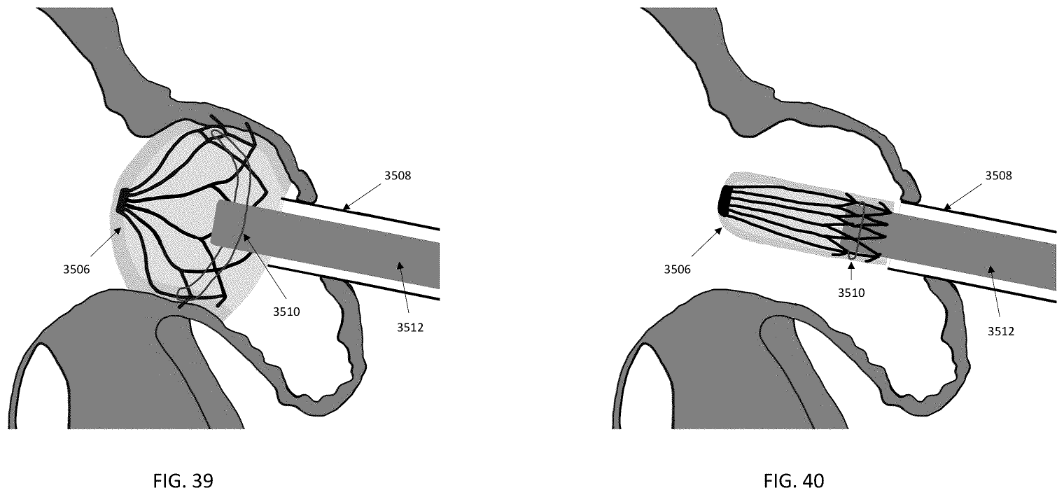

In some embodiments, the pusher 3512 may be advanced into the implant 3506 for implant collapse, as pictured in FIG. 39. Radial collapse of the implant 3506 may be actuated by suture tension pulling from the center of the implant 3506. The suture 3510 may be coupled to the end of the pusher 3512. As the pusher 3512 is inserted into the center of the implant 3506 and suture tension is applied, the implant 3506 collapses around the tip of the pusher 3512. The suture 3510 may be threaded through the frame of the implant 3506 so that when tension is applied, the frame collapses and the implant 3506 compresses.

FIG. 40 depicts the frame and implant 3506 collapsed around the distal tip of the trocar 3508. In this configuration, the implant 3506 may be repositioned within the LAA before deploying again. Alternatively, the trocar 3508 with the attached implant 3506 may be withdrawn from the LAA for complete removal and retrieval.

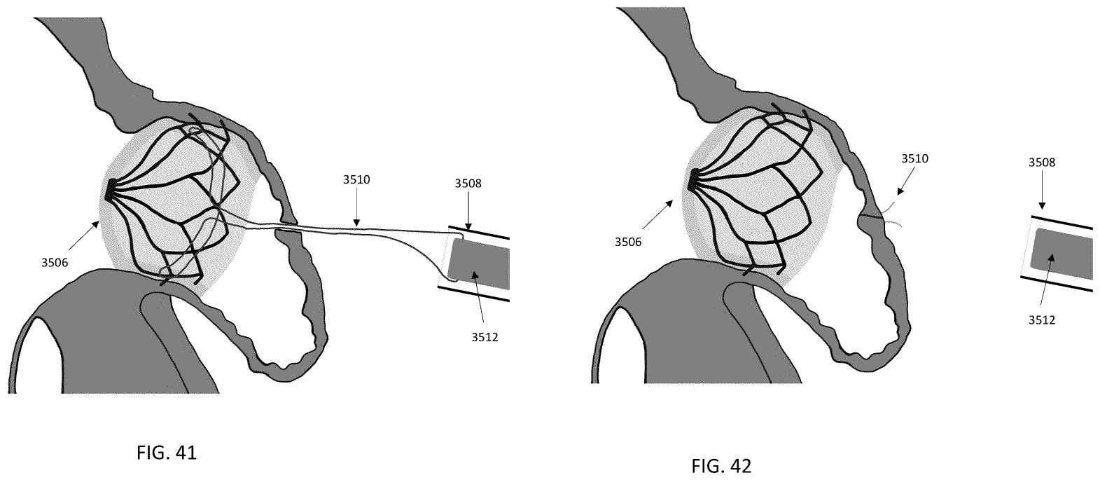

Once the implant is deployed into the desired position within the LAA, the delivery system may be removed through the incision, as depicted in FIG. 41. The suture 3510 may still be attached to both the frame of the implant 3506 and the pusher 3512 within the trocar 3508. In some embodiments, certain checks may be conducted to ensure the implant 3506 is situated as intended. Once the checks are complete, the delivery system may be removed from the body. For example, certain imaging modalities may be utilized to inspect position of the implant 3506. In another example, pushing on the implant 3506 may help verify positioning.

FIG. 42 depicts the delivery system and suture attachment 3510 being removed from the LAA. In some embodiments, once the positioning of the implant 3506 is checked, the suture attachment 3510 may be severed and removed from the frame of the implant 3506. The trocar 3508 can be removed from the body. The incision site in the LAA may be closed and the surgery completed.

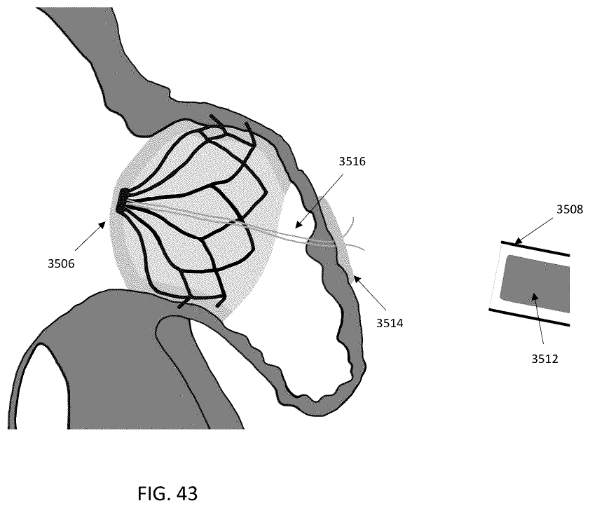

In an alternative embodiment, the implant 3506 may be additionally secured by a pledget mechanism 3514 outside the LAA as depicted in FIG. 43. As the incision of the LAA is closed, a suture 3516 connected to the implant may be fastened to the outer wall of the LAA for additional security. The addition of the pledget 3514 may prevent embolization of the implant 3506. In some embodiments, a tulip-style or flared inner tube can be used to assist with retrieval. A similar mechanism was described in relation to FIG. 34.

### E. Grappling Hook

Similar to the purse-string lasso concept, the grappling hook concept is a method to deliver an implant 4402 (such as device 3000) percutaneously through a transseptal puncture approach, however, via thorascopic or a direct surgical approach. In this approach, a grappling hook style device 4404 with fingers 4406 (e.g. hooks) is used to attach to the edges of the internal metal frame 4408. In some embodiments, such as when operating on a cold heart, the grappling hook device 4404 can be utilized to assist in expansion of the metal frame 4408.

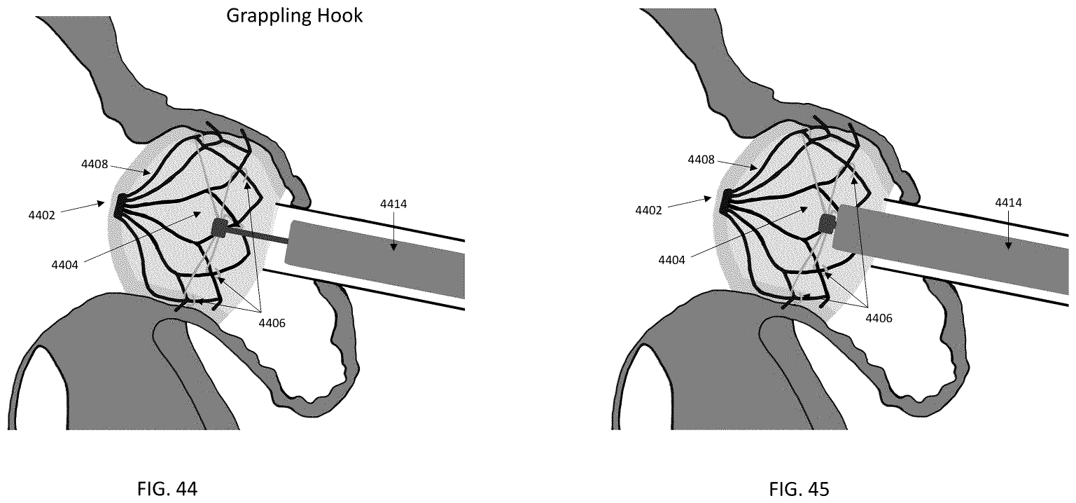

FIGS. 44-47 depict various features that may be used, either alone or in combination, with any of the LAA occlusion devices and methods described herein. In particular, FIGS. 44-47 show sequential views of an embodiment of a process for delivering an implant 4402 using a grappling hook technique. The device used for the grappling hook approach described with respect to FIGS. 44-47 may include any features of the devices shown and described with respect to FIGS. 3A-8D or 35-43.

FIG. 44 depicts deployment of an implant 4402 (such as device 3000) from the LAA tip. The implant frame 4408 can be attached to the delivery system. Attachment of the frame 4408 to the delivery system may occur through grappling hooks 4406 coupled to the apexes of the frame. The grappling hooks 4406 can couple to the frame 4408 via mechanical means, for example, curling around the frame 4408 by an open hook, a closed hook with an eyelet, a loop, etc.

In some embodiments the grappling hook 4406 may not be removed after deployment. After deployment of the implant 4402 with the grappling hook 4404, the shaft 4410 may be unscrewed from the central hub 4412. The hooks 4406 may be detached from the delivery system and left with the implant 4402.

FIG. 45 depicts the first step if the implant 4402 needs to be collapsed for repositioning. The pusher 4414 can be advanced up to the tip of the grappling hook assembly 4404. The pusher 4414 may be advanced into the center of the implant 4402.

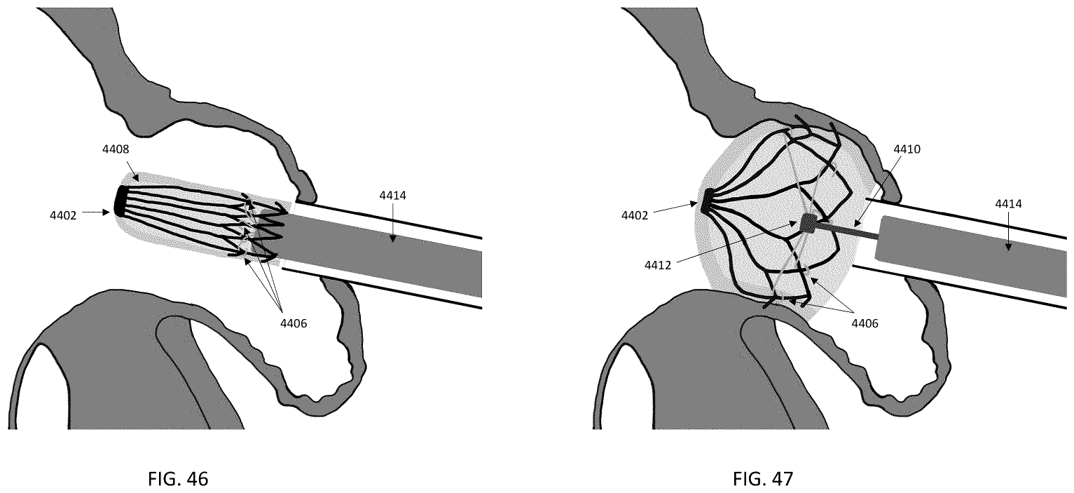

FIG. 46 depicts the implant 4402 collapsed around the pusher 4414 for repositioning or removal.

FIG. 47 depicts an alternative embodiment for attaching the grappling hooks 4406 to the implant 4402. In this embodiment, the grappling hooks 4406 are spring elements which can act to expand the implant frame 4402. The radial elements may be spring-like material that can apply outward radial force to the frame or inner walls of the implant 4402. The spring-like material can be made of stainless steel, CoCr, or other metal that would have ample stiffness to expand the implant 4402 even in a cold environment. This embodiment can be useful for deployment of Nitinol in a cold heart. A Nitinol frame may not expand well in a cold environment, so the grappling hook assembly 4404 may be implemented to assist in deployment.

### F. Inversion Resheath

The inversion resheath concept can also be utilized to deliver an implant 4802 (such as device 3000) percutaneously through a transseptal puncture approach, however, via thorascopic or a direct surgical approach. The implant 4802 can be attached to the delivery catheter via a suture or other method known on the art. If recapture is desired, a pusher 4806 can be advanced within the implant 4802 to the central hub 4808, the point of attachment, and the implant 4802 can be inverted for removal.

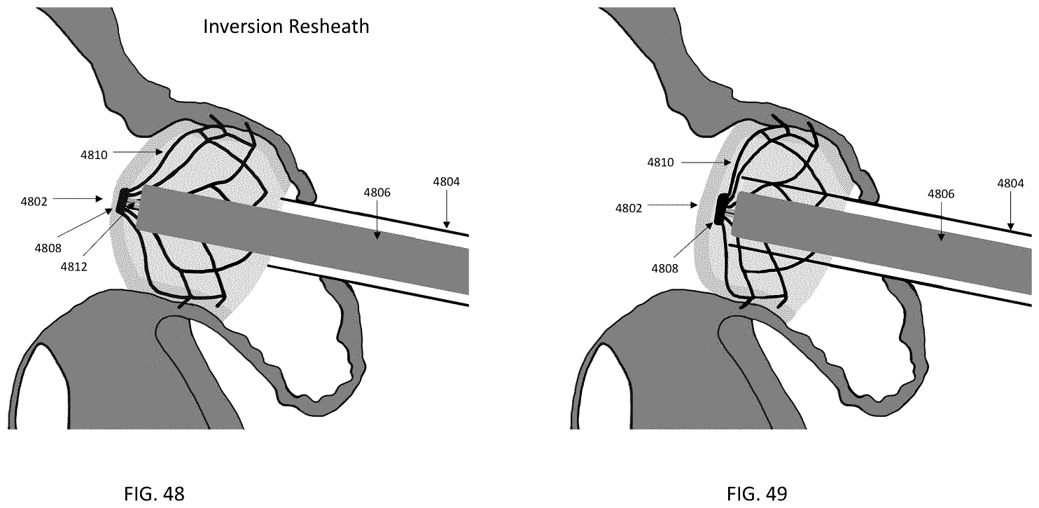

FIGS. 48-50 depict various features that may be used, either alone or in combination, with any of the LAA occlusion devices and methods described herein. In particular, FIGS. 48-50 show sequential views of an embodiment of a process for delivering an implant 4802 using an inversion resheath. The device used for the inversion resheath approach described with respect to FIGS. 48-50 may include any features of the devices shown and described with respect to FIGS. 3A-8D or 35-47.

FIG. 48 depicts a procedure for implantation of an implant 4802 (such as device 3000) from the distal tip of the LAA. Resheathing of the implant 4802 may be required after deployment. Resheathing can be accomplished by inverting the implant. In FIG. 48, the pusher 4806 may be placed adjacent to the LA tip from within the frame 4810.

In FIG. 49, the outer trocar 4804 can be placed within the frame 4810, coaxial to the pusher.

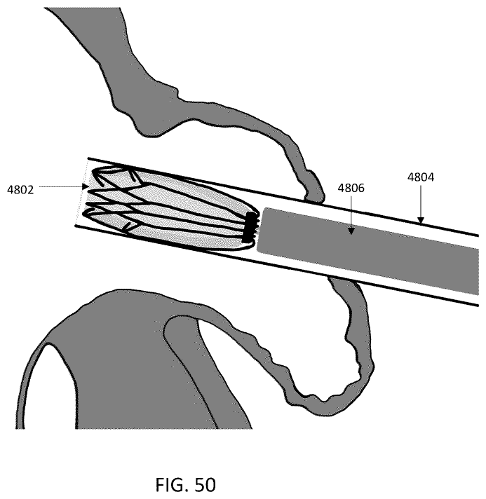

FIG. 50 depicts the implant 4802 inverted for repositioning or retrieval. The implant 4802 can be held by a suture 4812 from the back. Inversion may occur when the suture 4812 is pulled into the trocar and the implant 4802 inverts. The implant 4802 may invert similar to an umbrella being pulled into a smaller opening without first closing. Inversion may occur when pushing forward on the trocar 4804 and pulling back on the suture 4812 at the same time.

### G. Marshmallow Concept

The marshmallow concept consists of delivery through a trocar 5102 of a conformable soft foam or elastomeric cylindrical plug 5104 with an attachment point on the side adjacent to the LAA inner wall/tip. Following implantation, the plug 5104 can then be anchored in place either through the side wall of the appendage, using sutures, and/or via a suture attached to a pledget outside the LAA tip, which can also be utilized to close the entry site. In some embodiments, the marshmallow design may not have a frame. The implant 5104 can expand on its own because it is a solid plug made of foam instead of a cup. When deployed, the marshmallow implant 5104 can spring back to its original diameter or as constrained by the anatomy at the implant site.

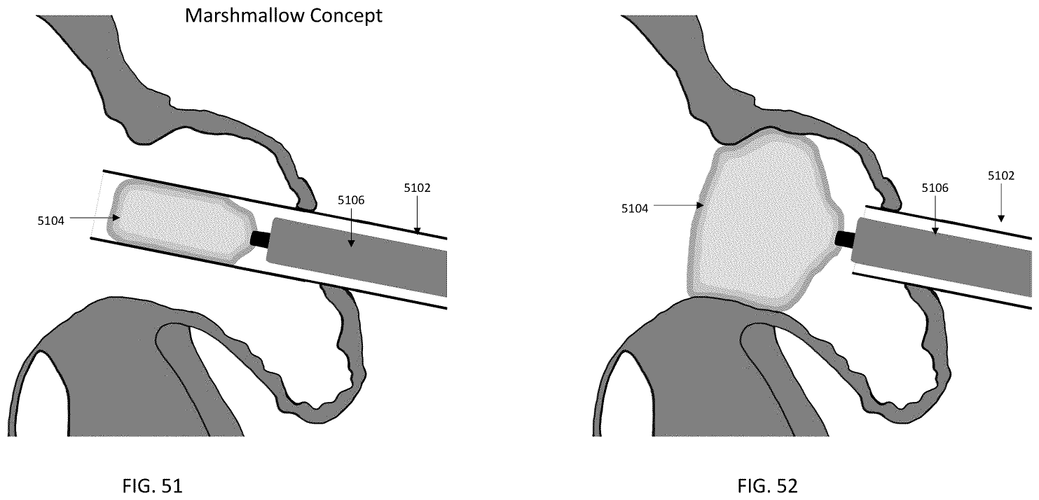

FIGS. 51-54 depict various features that may be used, either alone or in combination, with any of the LAA occlusion devices and methods described herein. In particular, FIGS. 51-54 show sequential views of an embodiment of a process for delivering a marshmallow-like implant through a delivery system.

FIG. 51 depicts a marshmallow-shaped LAA closure implant 5104 being delivered through a trocar 5102 from the distal LAA tip.

FIG. 52 depicts advancement of the pusher 5106 through the delivery system. As the pusher 5106 is advanced, the closure implant 5104 may exit the delivery system and expand.

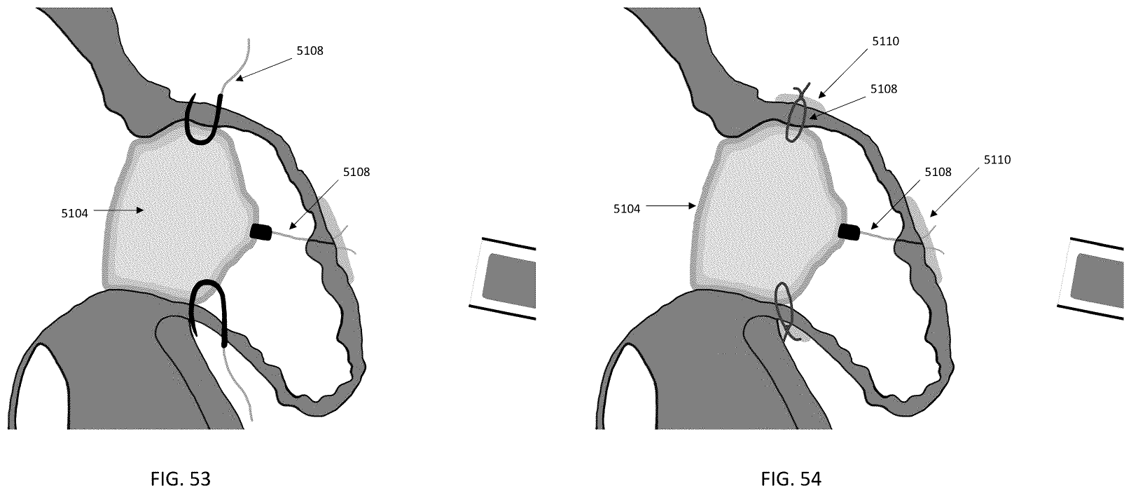

FIG. 53 depicts one embodiment for securing the implant 5104 within the LAA after deployment of the implant 5104 and removal of the trocar 5102. This embodiment includes suturing through foam near the ostium of the LAA, incorporating a tail tether 5108, or a combination of anchoring methods. Additionally, pledgets 5110 can be added outside the LAA for extra security.

FIG. 54 depicts the final deployed implant 5104, secured in place by sutures.

H. Implant with Modified Frame—Dual Hubs

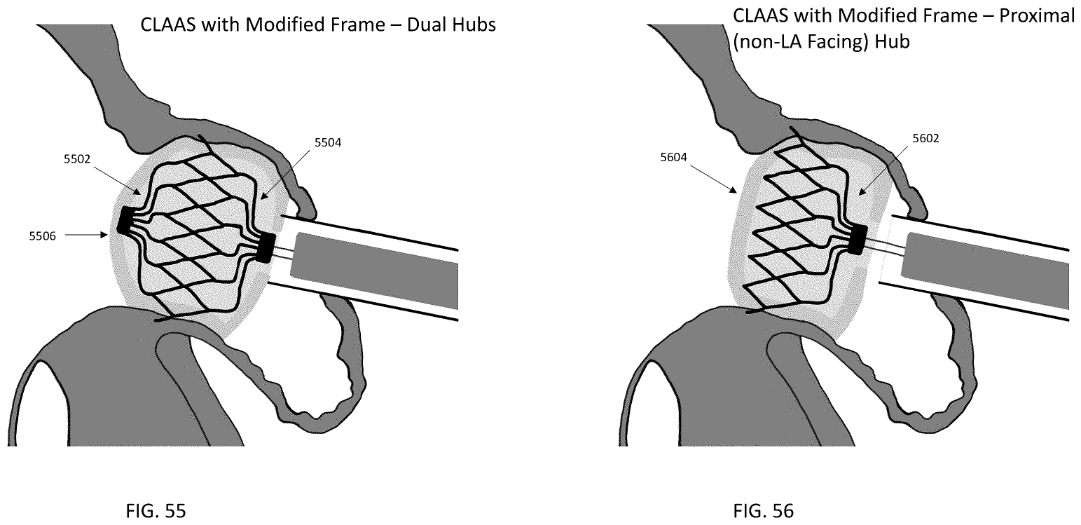

The dual hub embodiment in FIG. 55 has one hub 5502 that faces the LA and provides a flat, smooth surface. There may be a second hub 5504 facing the LAA tip that can be utilized to facilitate repositioning and recapture. The device used for the dual hub embodiment described with respect to FIG. 55 may include any features of the devices shown and described with respect to FIGS. 3A-8D or 35-54.

FIG. 55 depicts an embodiment of an implant 5506. The open distal end of an implant 5506, such as device 3000, is replaced by a second hub 5504. This allows the first hub 5502—positioned near the LAA ostium—to provide a flat surface in the LAA while the second hub 5504—positioned near the LAA tip—allows for attachment during delivery and repositioning or retrieval. The anchors can be similar to those of other embodiments described herein, such as device 3000. After deployed, the implant 5506 protruding into the LA may disrupt flow of blood in the LA. Disruption of blood flow can increase probability of thrombus formation, so a flat face on the implant may be preferable.

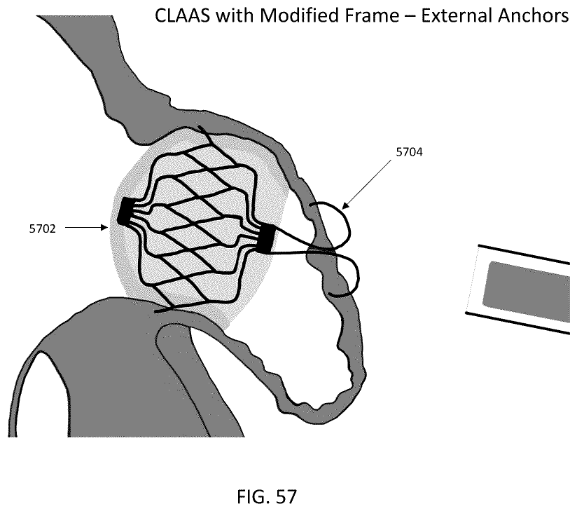

In some embodiments, a flat face can also be formed with the 1 hub facing away from the LA (see FIG. 56). One potential benefit of 2 hubs is that the radial elements protruding from the hub on the LA-facing side may help to maintain a more effective seal at the ostium.

I. Implant with Modified Frame—Proximal (Non-LA Facing) Hub

The proximal (non-LA facing) hub embodiment may have a hub 5602 facing the LAA tip to help facilitate repositioning and recapture. The proximal LA face can be just the tissue scaffold. The device used for the proximal hub embodiment described with respect to FIG. 56 may include any features of the devices shown and described with respect to FIGS. 3A-8D or 35-55.

In all of these embodiments, the tissue scaffold can be fabricated from a foam material, ePTFE, PET or another biocompatibile material. It can also be a composite of foam covered with a material such as ePTFE or PET.

FIG. 56 depicts a modified implant 5604 (such as device 3000) where the hub of the internal frame 5602 does not face the left atrium (LA). It faces the LAA tip to facilitate delivery and repositioning or retrieval. The tip of the frame 5602 is splayed outward to facilitate anchoring of the implant 5604. The open end of the frame 5602 is covered by foam which can also have an ePTFE cover, with or without perforations. If the implant 5604 is left open, thrombus could form inside it and then escape causing stroke. Closing it off would prevent thrombus forming inside the implant, or rather it would trap it so it can't come loose and cause stroke.

J. Implant with Modified Frame—External Anchors

The external anchors embodiment is an anchoring concept which can be used with any of the designs described in this disclosure. The anchors are attached to the LAA tip side of the implant 5702 and exit through the LAA wall, securing the implant 5702 in place.

FIG. 57 depicts an embodiment of the dual-hub implant 5702 with external anchors 5704. In this embodiment, a distal anchor 5704 that extends outside the LAA tip is employed. This type of anchor can be added to any of the implant designs in this disclosure. The external anchor 5704 can be applied to multiple different embodiments. As pictured in FIG. 57, the external anchors 5704 can be applied to a double-hub frame embodiment that was previously disclosed in FIG. 55.

The device used for the external anchor embodiment described with respect to FIG. 57 may include any features of the devices shown and described with respect to FIGS. 3A-8D or 35-56.

### K. Champagne Cork

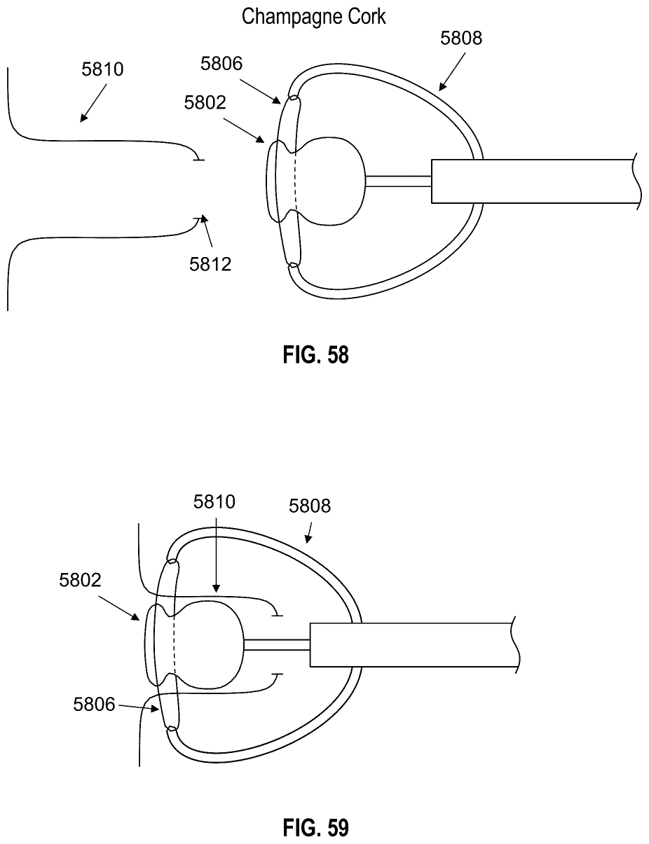

The champagne cork concept depicted in FIGS. 58-61 is designed to be implanted in a cold heart. The tip of the LAA 5810 is slit and the cork 5802 is positioned in the appropriate spot in the LAA through the use of the external alignment device 5804. This device has a suture 5806 that encircles the exterior of the LAA which is attached to metal arms 5808 for positioning. The arms 5808 contact the exterior of the heart to help position the external suture 5806 around the cork implant 5802, fixing it in place. The implant 5802 is fabricated from an elastomer or a dense foam that can take odd shapes but resists being crushed by the suture. Following placement of the cork 5802 and securing it by tightening of the external suture 5806, the access point is sutured closed.

FIG. 58 depicts a slit 5812 placed in the distal LAA 5810 tip of a cold heart to open it up to allow delivery of the implant 5802. The implant 5802 is loaded into the delivery system.

FIG. 59 depicts placement of the implant 5802 in an expanded shape. Alignment arms 5808 on the delivery system help to find the proper location for the suture lasso 5806 which is placed around the implant 5802 from outside the LAA.

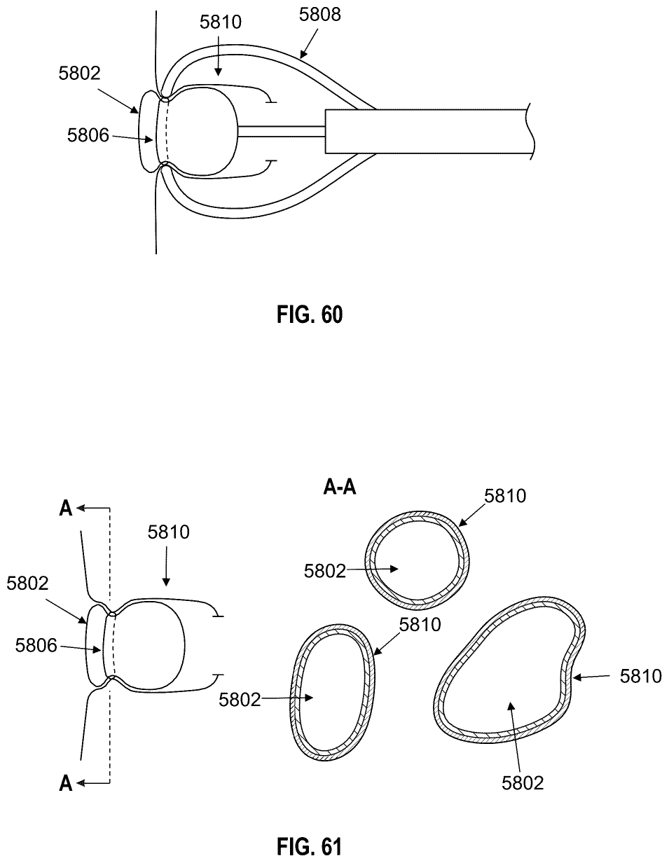

FIG. 60 depicts how this concept is different from other LAA closure methods which expand to fill a pressurized LAA. In this embodiment the LAA 5810 is brought down in contact with the implant 5802 from exterior to the LAA. The implant 5802 is fixed in place and can also bring anchors in touch with the interior LAA wall for additional fixation.

FIG. 61 depicts the final implant placement with the tip of the LAA 5810 sutured closed while also showing how the implant 5802 material is highly conformable to different shapes of LAA ostia.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination.

Similarly, while operations are depicted in the drawings in a particular order, this should not be understood as requiring that such operations be performed in the particular order shown or in sequential order, or that all illustrated operations be performed, to achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A device for closing off a left atrial appendage (LAA) of a patient, the device comprising:
a proximal hub;
one or more fronds extending distally from the proximal hub;
one or more anchors disposed at the distal end of the one or more fronds, the one or more anchors configured to engage an ostium of the LAA; and
a collar disposed around the one or more fronds, the collar configured to move distally along the one or more fronds causing the one or more anchors to be pulled toward a central longitudinal axis into a closed position and close the ostium of the LAA.

2. The device of claim 1, wherein the collar is configured to lock the one or more fronds in the closed position.

3. The device of claim 1, wherein the one or more fronds comprise a first bump feature.

4. The device of claim 3, wherein the collar is further configured to move distally along the one or more fronds and over the first bump feature, and wherein the first bump feature is configured to retain the collar in a distal position relative to the first bump feature.

5. The device of claim 3, wherein the one or more fronds comprise a second bump feature disposed distally from the first bump feature.

6. The device of claim 5, wherein the collar is further configured to:
move distally along the one or more fronds and over the first bump feature causing the one or more fronds to be pulled a first distance toward the central longitudinal axis and into a partially closed position; and
move distally along the one or more fronds and over the second bump feature causing the one or more fronds to be pulled a second distance toward the central longitudinal axis and into the closed position.

7. The device of claim 1, wherein the one or more fronds comprise at least two fronds or at least four fronds.

8. The device of claim 1, wherein the collar comprises a notch configured to secure the collar to a delivery device.

9. The device of claim 1, wherein the proximal hub comprises a pin configured to be disposed proximal to the collar and move distally into a central lumen of the collar, causing the one or more fronds to be deployed.

10. The device of claim 9, wherein the pin comprises a step configured to prevent the pin from extending beyond the collar.

11. A method for occluding a left atrial appendage (LAA) of a patient, the method comprising:
providing an implant within a delivery device, the implant comprising:
an expandable tubular body having a compressible open cell foam sidewall, an occlusive end, an open end, and a longitudinal axis extending therethrough; and
a self-expandable support carried within the expandable tubular body, the self-expandable support comprising a plurality of struts forming a plurality of apexes;
creating an incision at a tail of the LAA of the patient;
inserting the delivery device with the implant through the incision into the LAA;
deploying the implant within the LAA to allow the self-expandable support and the tubular body to expand such that the foam sidewall contacts the wall of the LAA and provides a cushion between the support and the wall of the LAA;
securing the implant; and
closing the incision at the tail of the LAA.

12. The method of claim 11, wherein the implant further comprises a suture tether attached to the self-expandable support, the suture tether configured to collapse the support when tension is applied to the suture tether.

13. The method of claim 12, wherein securing the implant comprises attaching the suture tether to the LAA.

14. The method of claim 11, wherein the delivery device comprises a grappling hook device, the grappling hook device comprising a plurality of hooks coupled to the plurality of apexes of the support.

15. The method of claim 14, wherein the grappling hook device further comprises a plurality of spring elements attached to the plurality of hooks, the plurality of spring elements configured to extend radially and expand the self-expandable support.

16. The method of claim 11 further comprising inverting the implant such that the occlusive end of the expandable tubular body passes through the open end of the expandable tubular body.

17. A method for occluding a left atrial appendage (LAA) of a patient, the method comprising:
providing an implant within a delivery device, the implant comprising a foam body;
creating an incision at a tail of the LAA of the patient;
inserting the delivery device with the implant through the incision into the LAA;
deploying the implant within the LAA;
securing the implant with the LAA; and
closing the incision at the tail of the LAA.

18. The method of claim 17, wherein the foam body comprises a solid plug configured to self-expand and occlude the LAA.

19. The method of claim 17, wherein the implant further comprises:
a first support disposed within the foam body, the first support comprising a first plurality of struts forming a first plurality of apexes; and
a second support disposed within the foam body, the second support comprising a second plurality of struts forming a second plurality of apexes.

20. The method of claim 17, wherein the implant further comprises a suture that encircles an exterior of an ostium of the LAA and secures the implant within the ostium of the LAA, and wherein the foam body comprises a cork shape configured to be placed in the ostium of the LAA and resist compression by the suture.
